SEQUENCE LISTING (23 sequences)

SEQ ID NO: 1 - protein, 15 aa, Artificial Sequence
  Synthesized
GGGGSGGGGSGGGGS

SEQ ID NO: 2 - protein, 4 aa, Artificial Sequence
  Synthesized
  misc_feature (2)..(3): Xaa can be any naturally occurring amino acid
YXXQ

SEQ ID NO: 3 - protein, 4 aa, Artificial Sequence
  Synthesized
YRHQ

SEQ ID NO: 4 - protein, 4 aa, Artificial Sequence
  Synthesized
  misc_feature (2)..(3): Xaa can be any naturally occurring amino acid
YXXL

SEQ ID NO: 5 - protein, 286 aa, Artificial Sequence
  Synthesized
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 6 - protein, 504 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHH

SEQ ID NO: 7 - protein, 264 aa, Artificial Sequence
  Synthesized
MALPVTALLLPLALLLHAARPQSVLTQPASVSGSPGQSVTISCTGTSSDVGGYNYVSWYQQHPGKPPKLMIYEVSKRPSGVPDRFSGSKSGNTASLTISGLQAEDEADYYCSSHTSSNTLIFGGGTKVTVLGGGGSGGGGSGGGGSEVQLVQSGAEVKKPGESLKISCKGSGYTFTNYWIGWVRQMPGKGLEWMGRIYPSDSYTNYSPSFQGQVTISADKSISTAYLQWSSLKASDTAMYYCARDLEPTHHYSWGQGTLVTVSS

SEQ ID NO: 8 - protein, 242 aa, Artificial Sequence
  Synthesized
DIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIKGGGGSGGGGSGGGGSEVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSS

SEQ ID NO: 9 - protein, 485 aa, Artificial Sequence
  Synthesized
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 503 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSHHHHHH

SEQ ID NO: 11 - protein, 504 aa, Artificial Sequence
  Synthesized
QVQLVQSGAEVKKPGASVKVSCKASGYTFTNHIIHWVRQAPGQGLEWMGYINPYPGYHAYNEKFQGRATMTSDTSTSTVYMELSSLRSEDTAVYYCARDGYYRDTDVLDYWGQGTLVTVSSGGGGSGGGGSGGGGSDIQMTQSPSSLSASVGDRVTITCQASQDISNYLNWYQQKPGKAPKLLIYYTSRLHTGVPSRFSGSGSGTDFTFTISSLEPEDIATYYCQQGNTLPWTFGQGTKVEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLHHHHHH

SEQ ID NO: 12 - protein, 498 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSQIVLTQSPAIMSASPGEKVTITCSVSSSVDYIHWFQQKPGTSPKFWIYSTSILASGVPARFSGSGSGTSYSLTISRMEAEDAATYYCQQRSSYPPTFGSGTKLEIKGGGGSGGGGSGGGGSQVQLQQSGGEVMKPGASVKLSCKATGYTFTGYWIEWIKQRPGHGLEWIGEILPGSGGTNYNEKFKGKATFTAHTSSNTAYMQLSSLTTEDSAIYYCARDSNSFTYWGQGTLVTVSSHHHHHH

SEQ ID NO: 13 - protein, 506 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSQSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGGGGGSGGGGSGGGGSEVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSHHHHHH

SEQ ID NO: 14 - protein, 497 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSQVKLQQSGPELVRPGASVKMSCKASGYTFTTYWMHWVKQRPGQGLEWIGMIDPSNSDTRLNQKFKDKATLNVDTSSNTAYMHLSSLTSEDSAVYYCTLGGTEYWGQGATLTVSSGGGGSGGGGSGGGGSSIVMTQSPATLSVTPGDRVSLSCRASQSIGDYLHWFQQKSHESPRLLIKYASQSISGIPSRFSGSGSGSDFTLIINTMEPEDVGVYYCQNAHTYPYTFGGGTKLEIKHHHHHH

SEQ ID NO: 15 - protein, 502 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSHHHHHH

SEQ ID NO: 16 - protein, 507 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKGGGGSGGGGSGGGGSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSHHHHHH

SEQ ID NO: 17 - protein, 504 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSQSVLTQPASVSGSPGQSVTISCTGTSSDVGGYNYVSWYQQHPGKPPKLMIYEVSKRPSGVPDRFSGSKSGNTASLTISGLQAEDEADYYCSSHTSSNTLIFGGGTKVTVLGGGGSGGGGSGGGGSEVQLVQSGAEVKKPGESLKISCKGSGYTFTNYWIGWVRQMPGKGLEWMGRIYPSDSYTNYSPSFQGQVTISADKSISTAYLQWSSLKASDTAMYYCARDLEPTHHYSWGQGTLVTVSSHHHHHH

SEQ ID NO: 18 - protein, 503 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIQMTQSPASLSASVGERVTITCRASGDIHNYLAWYQQKPGKSPQLLIYNAKTLADGVPSRFSGSGSGTQYTLTINSLQPEDFATYYCQHFWSFPWTFGGGTKLEIKGGGGSGGGGSGGGGSQVLVQSGAEVKKPGASVKVSCKASGYTFTDYNMDWVRQSHGQSLEWMGDINPNTGGTIFNQKFKGRATITVDTSSSTAYMDLHSLTSEDTAVYYCARRTYYGNSYYFDYWGQGTLVTVSSHHHHHH

SEQ ID NO: 19 - protein, 503 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIVLTQSPATLSVSPGERATLSCRASQGISDYLHWYQQKSGESPRLLIKYASQSMSGIPARFSGSGSGTDFTLTISSLEPEDVAVYYCQNGHTFPLTFGAGTRLEIKGGGGSGGGGSGGGGSQVQLVESGGGVVAPGQSLRLSCTASGFSLSSYGVHWVRQPPGKGLEWVAVIWAGGNTKYNSALMSRFTISRDTSKTTVYLQMNSLETEDTAMYYCATYFGSRHFYSMDYWGQGTLVTVSSHHHHHH

SEQ ID NO: 20 - protein, 502 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDIQMTQSPSSLSASVGDRVTITCRASQSISSYLNWYQQKPGKAPKLLIYAASSLQSGVPSGFSGSGSGTDFTLTISSLQPEDFATYYCQQSYSTPLTFGGGTKVEIKGGGGSGGGGSGGGGSQVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAREGKNGAFDIWGQGTMVTVSSHHHHHH

SEQ ID NO: 21 - protein, 502 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSEVQLVESGGGLVKPGGSLRLSCAASGFTYSNAWMSWVRQAPGKGLEWVGRIKSKTDGGTTDYAAPVQGRFTISRDDSKNTLYLQMNSLKTEDTAVYYCTIFGVVSFDYWGQGTLVTVSSGGGGSGGGGSGGGGSDVQMTQSPSSLSASVGDRVTITCRASQSISDYLSWYQQRPGKAPNLLIYAASNLKTGVPSRFSGSGSGTDFTLTISTLQPEDFATYYCQQSYRSPWTFGQGTKVEIKHHHHHH

SEQ ID NO: 22 - protein, 503 aa, Artificial Sequence
  Synthesized
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSDVVMTQSPLSLPVTPGEPASISCRSSQSLVHSNGNTYLHWYLQKPGQSPQLLIYKVSNRFSGVPDRFSGSGSGTDFTLKISRVEAEDVGVYYCSQNTHVPPTFGQGTKLEIKGGGGSGGGGSGGGGSQVQLVESGAEVKKPGASVKVSCKASGYTFTDYEMHWVRQAPGQGLEWMGALDPKTGDTAYSQKFKGRVTITADESTSTAYMELSSLRSEDTAVYYCARFYSYTYWGQGTLVTVSSHHHHHH

SEQ ID NO: 23 - protein, 20 aa, Artificial Sequence
  Synthesized
GGGGSGGGGSGGGGSGGGGS

(19) United States
(12) Patent Application Publication
(10) Pub. No.: US 2023/0201258 A1
(43) Pub. Date: Jun. 29, 2023

(54) Polyspecific Binding Molecules and their use in Cell Therapy

(71) Applicants: Innovative Cellular Therapeutics Holdings, Ltd., Rockville, MD (US); Innovative Cellular Therapeutics, Inc., Rockville, MD (US)

(72) Inventors: Chengfei Pu, Shanghai (CN); Lei Xiao, Rockville, MD (US); Jingrui Liu, Shanghai (CN); Junkai Peng, Shanghai (CN); Le Tian, Rockville, MD (US); Xiaogang Shen, Shanghai (CN); Zhiyuan Cao, Shanghai (CN); Zhao Wu, Shanghai (CN)

(73) Assignees: Innovative Cellular Therapeutics Holdings, Ltd., Rockville, MD (US); Innovative Cellular Therapeutics, Inc., Rockville, MD (US)

(21) Appl. No.: 17/996,589

(22) PCT Filed: Apr. 21, 2021
(86) PCT No.: PCT/US2021/028429

§ 371 Date: Oct. 19, 2022

Related U.S. Application Data
(63) Continuation of application No. 16/999,357, filed on Aug. 21, 2020.
Application No. 16/999,357 is a continuation-in-part of application No. 16/996,237, filed on Aug. 18, 2020.
(60) Provisional application No. 63/070,682, filed on Aug. 26, 2020.
(60) Provisional application No. 63/054,017, filed on Jul. 20, 2020.
(60) Provisional application No. 63/046,859, filed on Jul. 1, 2020.
(60) Provisional application No. 63/040,851, filed on Jun. 18, 2020.
(60) Provisional application No. 63/035,322, filed on Jun. 5, 2020.
(60) Provisional application No. 63/014,379, filed on Apr. 23, 2020.

(51) Int. Cl. A61K 35/17; A61P 35/00; A61K 39/395

(52) U.S. Cl. CPC A61K 35/17; A61P 35/00; A61K 39/39541; A61K 2039/505

(57) ABSTRACT

The present disclosure relates to compositions and methods for enhancing cell response and/or expanding chimeric antigen receptor (CAR) cells and/or maintenance in vivo and/or in vitro. In embodiments, the method comprises obtaining CAR T cells comprising a CAR comprising a binding domain that binds a solid tumor antigen, a transmembrane domain, and an intracellular domain; and contacting the CART cells with white blood cells and a bispecific antibody, such as a Bispecific T cell engager (BiTE®), thereby activating the CAR T cells, wherein the level of activation of the CAR T cells is higher than the level of activation of CAR T ells that are contacted with B cells without the bispecific antibody. The bispecific antibody comprises a first binding domain binding CD3 and a second binding domain binding CD19, CD20, CD22, or BCMA.

### CROSS REFERENCE TO RELATED APPLICATIONS

This application claims the benefit of U.S. Provisional Application No. 63/070,682, filed Aug. 26, 2020; U.S. application Ser. No. 16/999,357, filed Aug. 21, 2020; U.S. application Ser. No. 16/996,237, filed Aug. 18, 2020; U.S. Provisional Application No. 63/054,017, filed Jul. 20, 2020; U.S. Provisional Application No. 63/046,859, filed Jul. 1, 2020; U.S. Provisional Application No. 63/040,851, filed Jun. 18, 2020; U.S. Provisional Application No. 63/035,322, filed Jun. 5, 2020; and U.S. Provisional Application No. 63/014,379, filed Apr. 23, 2020; all of which are incorporated herein by reference in their entirety.

### SEQUENCE LISTING INFORMATION

A computer readable textfile, entitled “SDS1.0094PCT_ST25.txt,” created on or about Apr. 21, 2021 with a file size of about 73 KB, contains the sequence listing for this application and is hereby incorporated by reference in its entirety.

### TECHNICAL FIELD

The present disclosure relates to compositions and methods for expanding and maintaining modified cells including genetically modified cells and uses thereof in the treatment of diseases, including cancer.

### BACKGROUND

Cancer is characterized by abnormal growth of cells that invade or spread to other parts of the body. In humans, there are more than one hundred types of cancer. One example is breast cancer occurring in the epithelial tissue of the breast. Since breast cancer cells lose normal cells' characteristics, the connection between breast cancer cells is lost. Once cancer cells are exfoliated, they spread over the entire body via the blood and/or lymph systems and become life-threatening. Currently, breast cancer has become one of the common threats to women's physical and mental health. Although immunotherapy (e.g., chimeric antigen receptor T (CAR T) cell therapy) has been proven to be useful for treating some cancers, there is still a need to improve immunotherapy to treat more cancers including those involving solid tumors.

### SUMMARY

The present disclosure relates to compositions and methods for enhancing T cell response and/or CAR cell expansion and/or maintenance in vivo and/or in vitro. In embodiments, the present disclosure describes a method including obtaining modified cells comprising a binding molecule that binds a solid tumor antigen; contacting the modified cells with a population of cells comprising an antigen of a white blood cell (WBC) and a polyspecific binding molecule (PBM), the PBM comprising a first binding domain binding a T cell and a second binding domain binding the antigen of the WBC; and allowing the modified cells to expand and/or to be activated. In embodiments, the level of expansion and/or activation of the modified cells is higher than the level of expansion and/or activation of the modified cells contacted with the population of cells without the PBM.

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 shows forms of PBMs (polyspecific binding molecules, e.g., polyspecific antibodies).

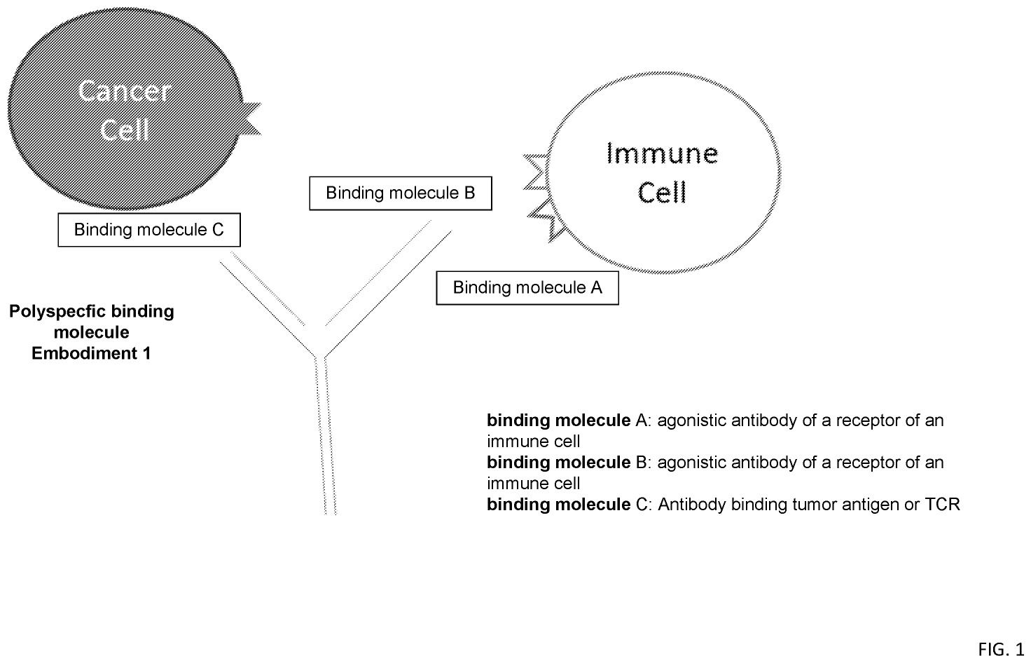

FIG. 2 shows forms of PBMs and their uses.

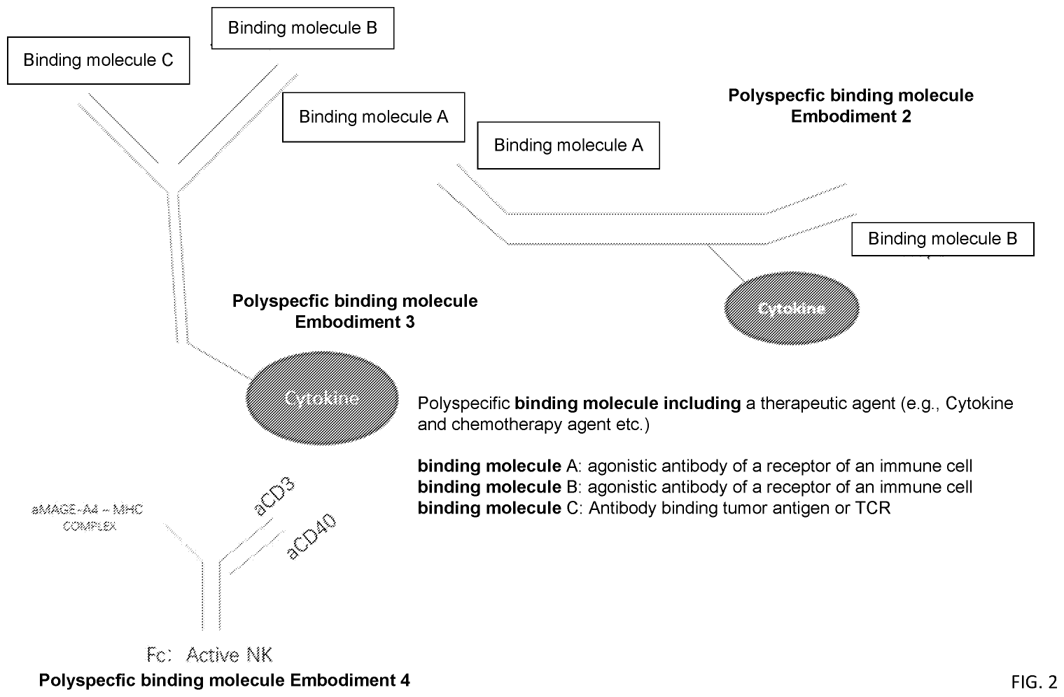

FIG. 3 shows examples of PBMs and their uses (A, B, and C refer to binding molecules A, B, and C in FIG. 2, respectively).

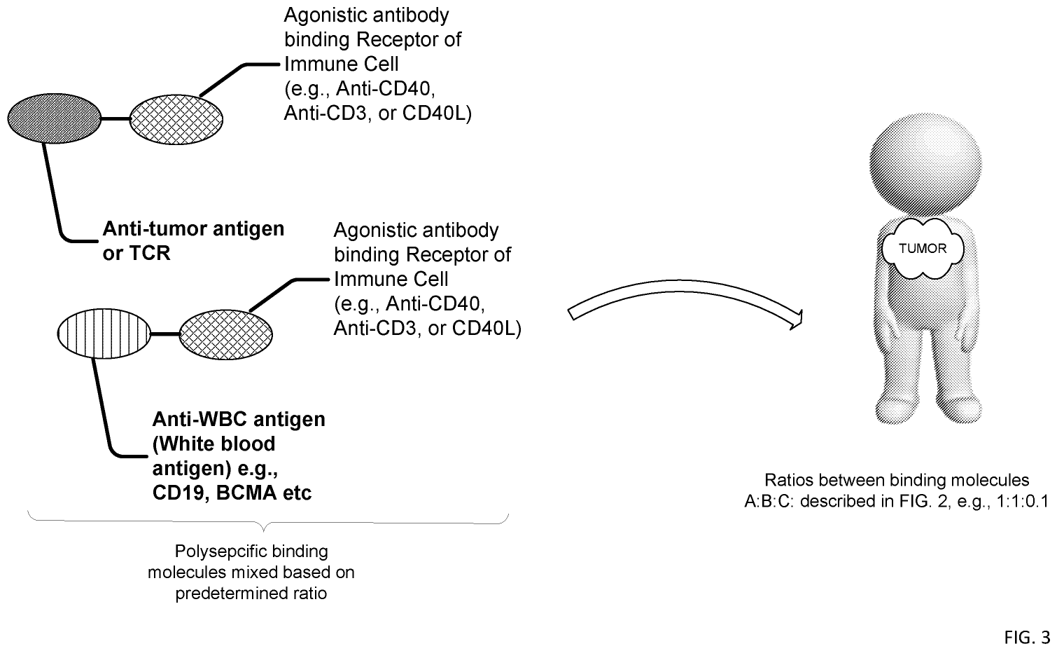

FIG. 4 shows structures of a CAR and a corresponding PBM.

FIG. 5 shows structures of a CAR and a corresponding PBM.

FIG. 6 shows structures of a CAR and a corresponding PBM.

FIG. 7 shows structures of a CAR and a corresponding PBM as well as uses thereof.

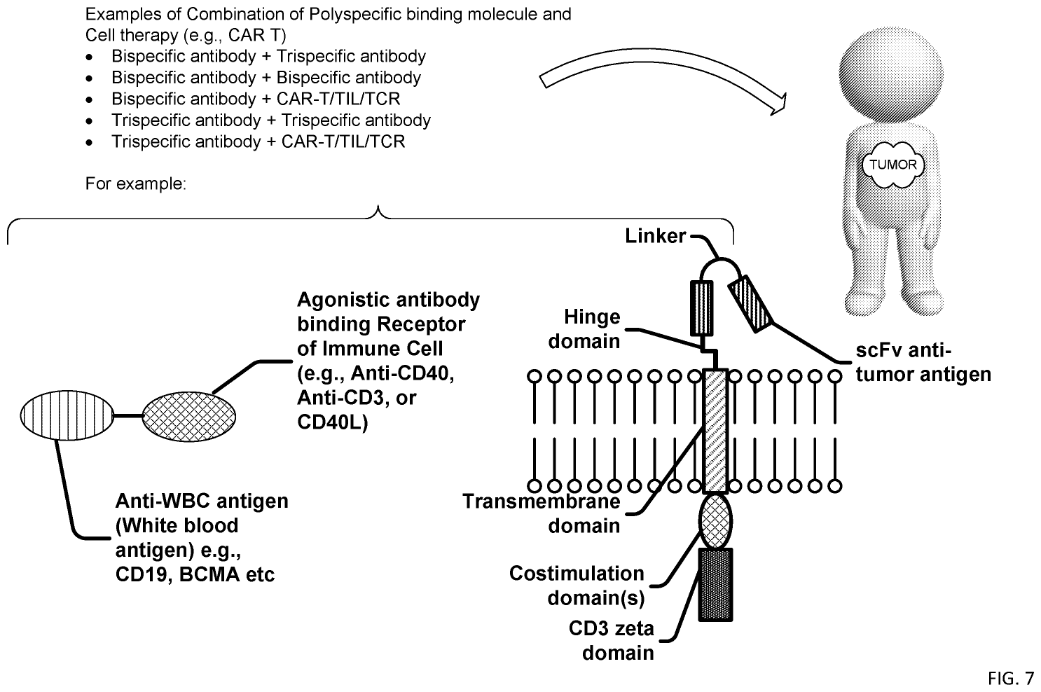

FIG. 8 shows schematic structures of vectors.

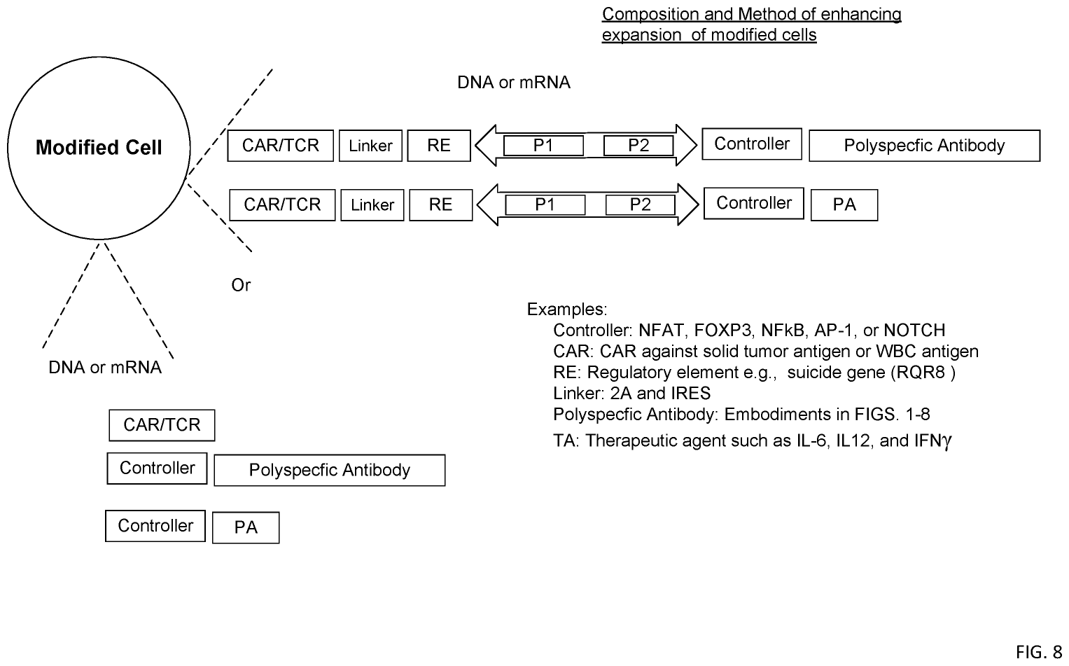

FIG. 9 shows a schematic example of cancer treatment using Tumor-Infiltrating Lymphocyte (TIL) techniques.

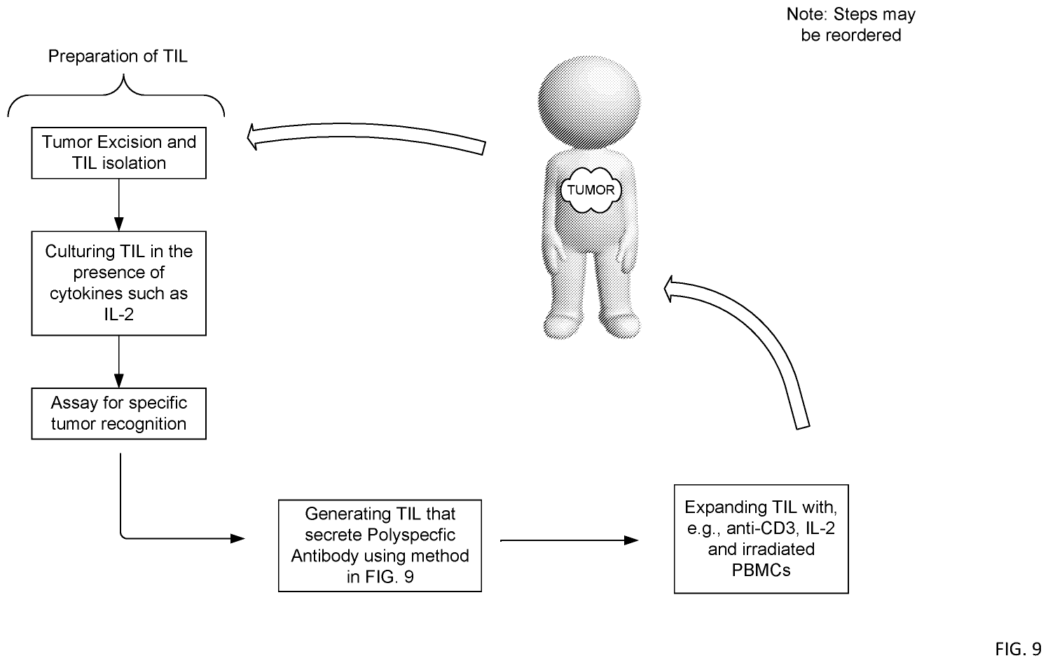

FIG. 10 shows a schematic example of cancer treatment using PBMs.

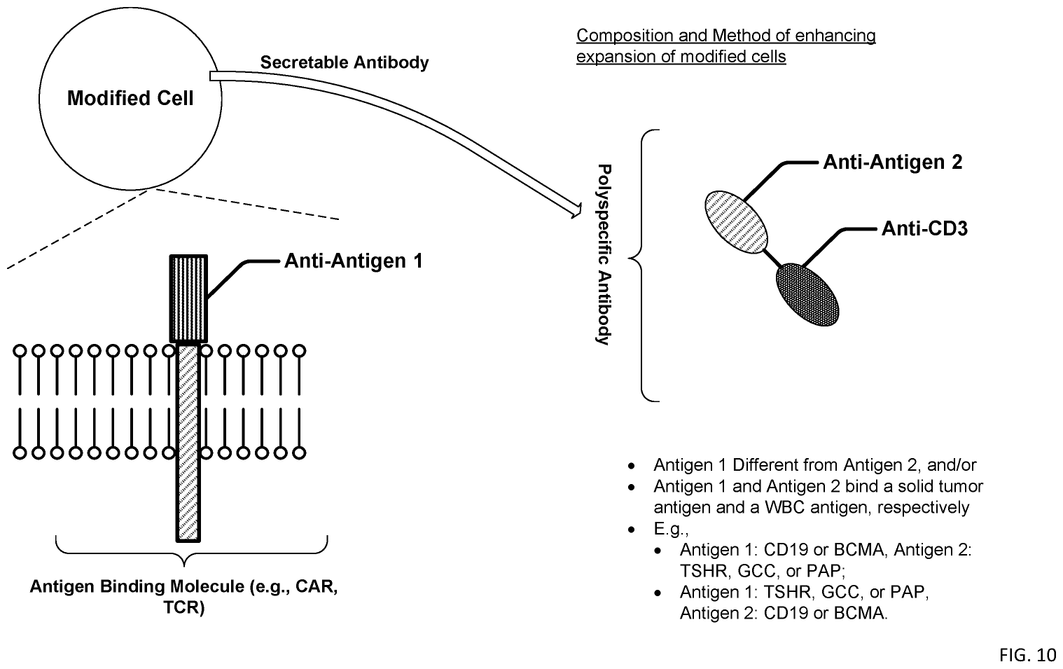

FIG. 11 shows a schematic example of cancer treatment using PBMs.

FIG. 12 shows a schematic example of cell expansion using PBMs.

FIG. 13 shows a schematic example of cell expansion using PBMs.

FIG. 14 shows a schematic example of cell expansion using PBMs.

FIG. 15 shows a schematic example of cell expansion using PBMs.

FIG. 16 shows a schematic example of cell expansion using PBMs.

FIG. 17 shows a schematic example of cell expansion using PBMs.

FIG. 18 shows a schematic example of cell expansion using lymphocytes expressing antigens.

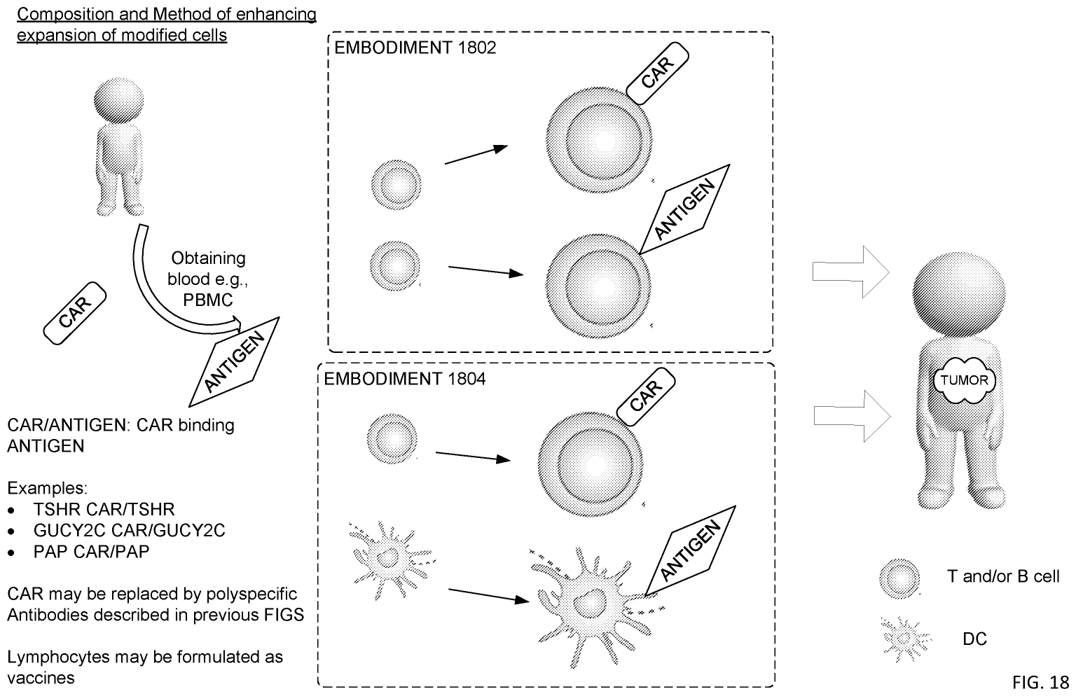

FIG. 19 shows an example of the enhancement of in vivo cell expansion.

FIG. 20 shows an example of enhancement of in vivo cell expansion.

FIG. 21 shows an example of enhancement of in vivo cell expansion.

FIG. 22 shows an example of enhancement of in vivo cell expansion with a safety device (Tag-anti-CD40 or CD40L). Examples of JAK-STAT pathway activating domain include IL2RB chain fragments (Kim et al., Biomolecules 2020, 10:263) and YXXQ (SEQ ID NO: 2; Kagoya et al. Nat. Med. 2018, 24(3): 352-359).

FIG. 23 shows an example of enhancement of in vivo cell expansion. Examples of JAK-STAT pathway activation domain includes IL2RB chain fragments (Kim et al., Biomolecules 2020, 10:263) and JAK-STAT domain (Kagoya et al. Nat. Med. 2018, 24(3): 352-359).

FIG. 24 shows an example of enhancement of in vivo cell expansion.

FIG. 25 shows an example of enhancement of in vivo cell expansion.

FIG. 26 shows compositions and methods of enhancing expansion of modified cells expansion and cell therapy. Additional information regarding the Fab can be found at Claus et al., Sci Transl Med. 2019, 11:496, which is incorporated in its entirety.

FIG. 27 shows a schematic overview of an implementation of bispecific T cell engagers (BiTEs) in CoupledCAR®.

FIG. 28 shows flow cytometry results of CAR expression.

FIG. 29 shows flow cytometry results of isolation of lymphocytes.

FIG. 30 shows flow cytometry results of in vitro assay.

FIG. 31 shows other flow cytometry results of the in vitro assay.

FIG. 32 shows a summary of the in vitro assay shown in FIGS. 30 and 31.

FIG. 33 shows flow cytometry results of in vitro assay.

FIG. 34 shows flow cytometry results of the in vitro assay.

FIG. 35 shows flow cytometry results of the in vitro assay.

FIG. 36 shows flow cytometry results of the in vitro assay.

FIG. 37 shows flow cytometry results of in vitro assay.

FIG. 38 shows cell expansion results of the in vitro assay.

FIG. 39 shows cell expansion results of the in vitro assay.

FIG. 40 shows a summary of the in vitro assay shown in FIGS. 38 and 39.

FIG. 41 shows the results of cytokine release assay. The results indicate that BiTEs enhanced cytokine release of T cells in cytokine release assay.

FIG. 42 shows flow cytometry results of in vitro assay.

FIG. 43 shows flow cytometry results of the in vitro assay.

FIG. 44 shows yet flow cytometry results of the in vitro assay.

FIG. 45 shows yet flow cytometry results of the in vitro assay.

FIG. 46 shows flow cytometry results of the in vitro assay.

FIG. 47 shows cell expansion results of in vitro assay.

FIG. 48 shows other cell expansion results of the in vitro assay.

FIG. 49 shows a summary of the in vitro assays shown in FIGS. 47 and 48.

FIG. 50 shows the results of cytokine release assay. The results indicate that BiTEs stimulated CART cells to release cytokines in the presence of B cells.

FIG. 51 shows a schematic overview of an immunotherapeutic system.

FIG. 52 shows a schematic overview of an implementation of the immunotherapeutic system in FIG. 51.

FIG. 53 shows an embodiment of PBMs.

FIG. 54 shows an embodiment of PBMs.

FIG. 55 shows an embodiment of PBMs.

FIG. 56 shows an embodiment of PBMs.

FIG. 57 shows an embodiment of PBMs.

FIG. 58 shows an embodiment of PBMs.

FIG. 59 shows an embodiment of PBMs.

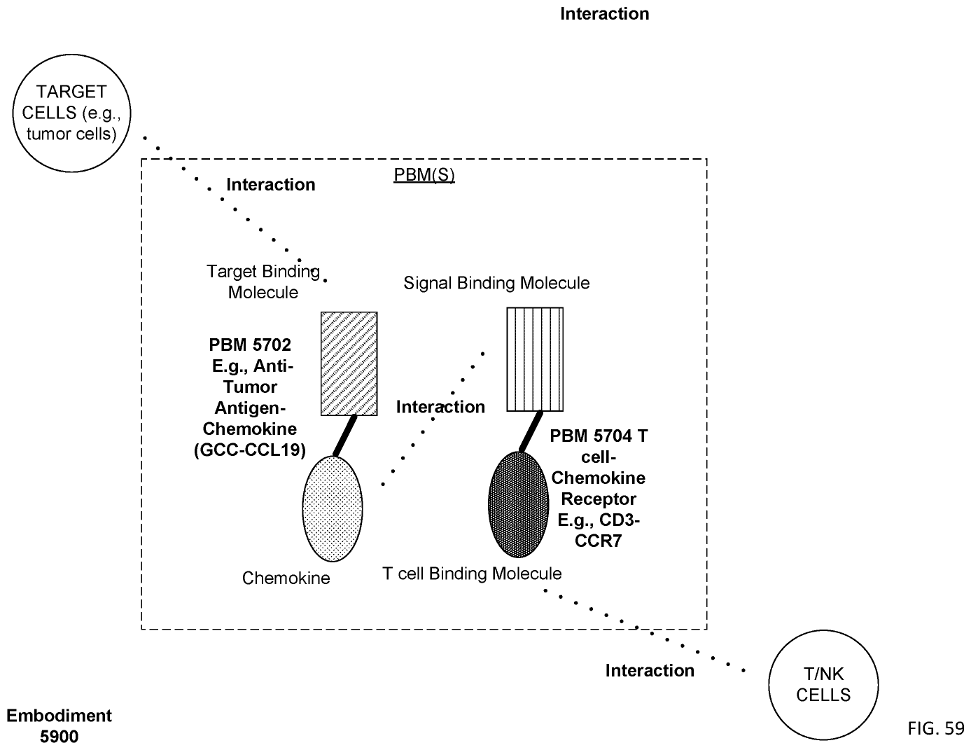

FIG. 60 shows modified cells including various target vectors.

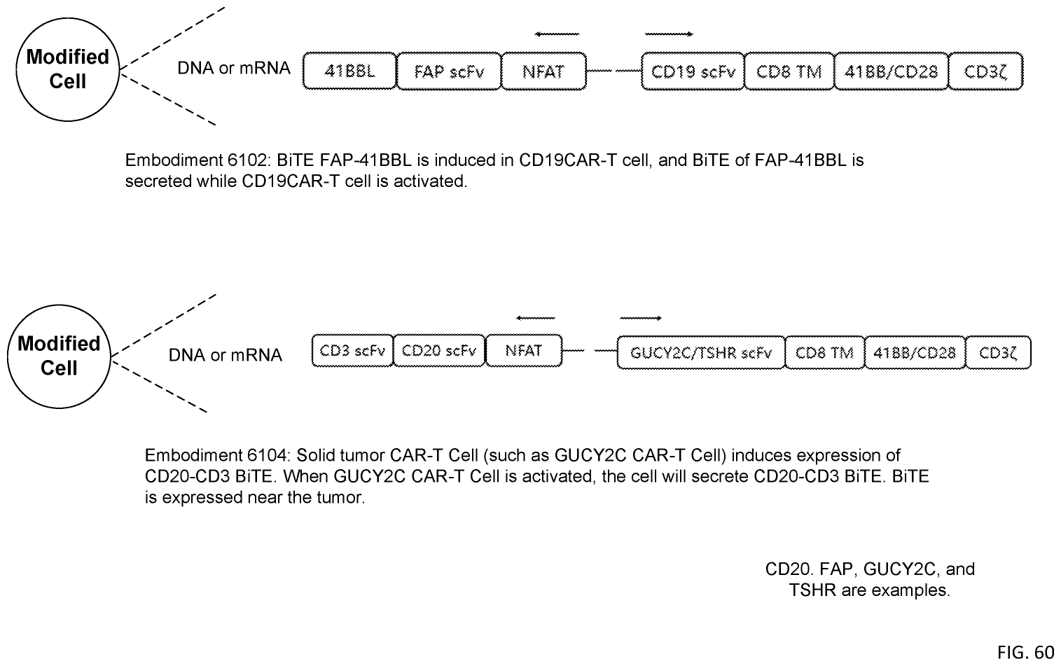

FIG. 61 shows schematic designs of combinations of PBMs to treat conditions.

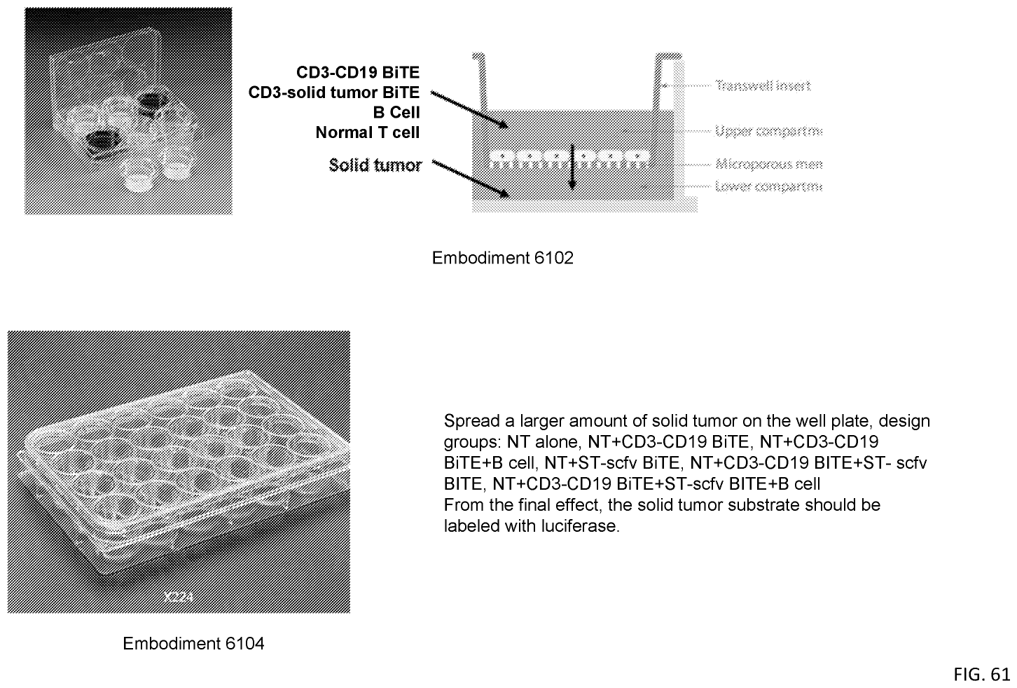

FIG. 62 shows IL-2 release in mice infused with CAR T cells and various agents.

FIG. 63 shows IFN-γ release in mice infused with CAR T cells and various agents.

FIG. 64 shows IL-4 release in mice infused with CAR T cells and various agents.

### DETAILED DESCRIPTION

A potential reason for the ineffective treatment of solid tumors using CAR T is the lack of timely activation of solid tumor CAR T cells. The solid tumor CAR T cells are not activated before exposure to solid tumor antigens. Because they are not activated, CAR T cells are not expanded. Therefore, they lack the ability to traffic to tumor sites. Embodiments in the present disclosure are designed and tested to resolve this problem.

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight, or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multispecific antibodies (e.g., bispecific antibodies), and antibody fragments so long as they exhibit the desired biological activity or function. The antibodies in the present disclosure may exist in a variety of forms including, for example, polyclonal antibodies; monoclonal antibodies; Fv, Fab, Fab′, and F(ab′)₂ fragments; as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragments” refers to a portion of a full-length antibody, for example, the antigen binding or variable region of the antibody. Other examples of antibody fragments include Fab, Fab′, F(ab′)₂, and Fv fragments; diabodies; linear antibodies; single-chain antibody molecules; and multispecific antibodies formed from antibody fragments.

The term “Fv” refers to the minimum antibody fragment which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. κ and λ light chains refer to the two major antibody light chain isotypes.

As used herein, a “polyspecific binding molecule” (PBM) or “multispecific binding molecule” refers to a molecule capable of binding more than one target, such as more than one antigen. Examples of the PBM may include a bispecific binding molecule and a trispecific binding molecule. Examples of the binding molecule may include an antibody, a receptor (e.g., TCR), a ligand, an agonist, a cytokine, etc. The binding molecules may be connected via various compositions such as a linker, a nanoparticle, a bead, a surface, etc. Additional information regarding bispecific and trispecific binding molecules can be found at: Runcie et al. Molecular Medicine (2018) 24:50 and, Oates J, Hassan N J, Jakobsen B K. ImmTACs for targeted cancer therapy: Why, what, how, and which. Mol Immunol. 2015; 67(2 Pt A):67-74. doi:10.1016/j.molimm.2015.01.024, all of which are incorporated by reference in their entirety. In embodiments, a PBM may include an antibody binding a T cell, a linker, and an antibody binding a solid tumor antigen. In embodiments, a PBM may include a TCR binding a TCR antigen (e.g., HLA-restricted peptide antigen) and an antibody binding a T cell. Examples of the TCR antigen include CEA, gp100, MART-1, p53, MAGE-A314, and NY-ESO-1.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumor in the first place.

The term “auto-antigen” refers to an endogenous antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the same subject.

The term “allogeneic” is used to describe a graft derived from a different subject of the same species. As an example, a donor subject may be a related or unrelated to the recipient subject, but the donor subject has immune system markers which are similar to the recipient subject.

The term “xenogeneic” is used to describe a graft derived from a subject of a different species. As an example, the donor subject is from a different species than a recipient subject, and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Throughout this specification, unless the context requires otherwise, the words “comprise,” “includes” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that no other elements may be present.

The phrase “consisting essentially of” is meant to include any element listed after the phrase and can include other elements that do not interfere with or contribute to the activity or action specified in the disclosure for the listed elements. Thus, the phrase “consisting essentially of” indicates that the listed elements are required or mandatory, and other elements may be present or optional. These other elements do not affect the activity or action of the required elements in a statistically significant manner, As an example, these other elements do not affect the ability of the required elements to kill cancer cells or to expand or maintain cells. These elements include excipients or carriers.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules, or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand,” refers to a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor, and a ligand that specifically binds with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T cell proliferation and/or upregulation or downregulation of key molecules.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression or overexpression” refers to the transcription and/or translation of a particular nucleotide sequence into a precursor or mature protein, for example, driven by its promoter. “Overexpression” refers to the production of a gene product in transgenic organisms or cells that exceeds levels of production in normal or non-transformed organisms or cells. As defined herein, the term “expression” refers to expression or overexpression.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

Viruses can be used to deliver nucleic acids into a cell in vitro and in vivo (in a subject). Examples of viruses useful for delivery of nucleic acids into cells include retrovirus, adenovirus, herpes simplex virus, vaccinia virus, and adeno-associated virus.

There also exist non-viral methods for delivering nucleic acids into a cell, for example, electroporation, gene gun, sonoporation, magnetofection, and the use of oligonucleotides, lipoplexes, dendrimers, and inorganic nanoparticles.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous, then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The term “substantially purified” refers to a material that is substantially free from components that are normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables integration of the genetic information into the host chromosome resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control (regulate) the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumor or a hematological malignancy characterized by overexpression of the tumor antigen can be determined by standard assays known in the art.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor antigen | Disease tumor |
|---|---|
| PRLR | Breast Cancer |
| CLCA1 | colorectal Cancer |
| MUC12 | colorectal Cancer |
| GUCY2C | colorectal Cancer |
| GPR35 | colorectal Cancer |
| CR1L | Gastric Cancer |
| MUC 17 | Gastric Cancer |
| TMPRSS11B | esophageal Cancer |
| MUC21 | esophageal Cancer |
| TMPRSS11E | esophageal Cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial cancer |
| UPK2 | Urothelial cancer (including bladder cancer) |
| ADAM12 | Breast cancer, pancreatic cancer and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL13-Rα2 | Glioma |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | carcinoma |
| EpCAM | carcinoma |
| EGFRvIII | Glioma-Glioblastoma |
| EGFR | Glioma-NSCL cancer |
| tMUC1 | Cholangiocarcinoma, Pancreatic cancer, |
| | Breast |
| PSCA | pancreas, stomach, or prostate cancer |
| FCER2, GPR18, FCRLA, | breast cancer |
| CXCR5, FCRL3, FCRL2, | |
| HTR3A, and CLEC17A | |
| TRPMI, SLC45A2, and | lymphoma |
| SLC24A5 | |
| DPEP3 | melanoma |
| KCNK16 | ovarian, testis |
| LIM2 or KCNV2 | pancreatic |
| SLC26A4 | thyroid cancer |
| CD171 | Neuroblastoma |
| Glypican-3 | Sarcoma |
| IL-13 | Glioma |
| CD79a or CD79b | Lymphoma |

The term “parenteral administration” of a composition includes, e.g., subcutaneous (s.c.), intravenous (i.v.), intramuscular (i.m.), intrasternal injection, or infusion techniques.

The terms “patient,” “subject,” and “individual,” and the like are used interchangeably herein and refer to any human, or animal, amenable to the methods described herein. In certain non-limiting embodiments, the patient, subject, or individual is a human or animal. In embodiments, the term “subject” is intended to include living organisms in which an immune response can be elicited (e.g., mammals). Examples of subjects include humans, and animals, such as dogs, cats, mice, rats, and transgenic species thereof.

A subject in need of treatment or in need thereof includes a subject having a disease, condition, or disorder that needs to be treated. A subject in need thereof also includes a subject that needs treatment for prevention of a disease, condition, or disorder, for example, cancer.

The term “polynucleotide” or “nucleic acid” refers to mRNA, RNA, cRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double-stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures.

The term “stimulatory molecule” refers to a molecule on a T cell that specifically binds a cognate stimulatory ligand present on an antigen presenting cell. For example, a functional signaling domain derived from a stimulatory molecule is the zeta chain associated with the T cell receptor (TCR) complex. The stimulatory molecule includes a domain responsible for signal transduction.

The term “stimulatory ligand” refers to a ligand that when present on an antigen presenting cell (e.g., an APC, a dendritic cell, a B-cell, and the like) can specifically bind with a cognate binding partner (referred to herein as a “stimulatory molecule”) on a cell, for example a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease, and its severity, and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one that has been transfected, transformed, or transduced with an exogenous nucleic acid. The cell includes the primary subject cell and its progeny.

The term “vector” refers to a polynucleotide that comprises an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate the transfer of nucleic acid into cells, such as, for example, polylysine compounds, liposomes, and the like. Examples of viral vectors include adenoviral vectors, adeno-associated virus vectors, retroviral vectors, and others. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted, making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, and 6. This applies regardless of the breadth of the range.

A “chimeric antigen receptor” (CAR) molecule is a recombinant polypeptide including at least an extracellular domain, a transmembrane domain, and a cytoplasmic domain or intracellular domain. In embodiments, the domains of the CAR are on the same polypeptide chain, for example, a chimeric fusion protein. In embodiments, the domains are on different polypeptide chains, for example, the domains are not contiguous.

The extracellular domain of a CAR molecule includes one or more antigen binding domains. The CAR molecule can be a polyspecific binding molecule, such as a bispecific CAR. Examples of bispecific chimeric antigen receptors (CARs) include a CAR binding CD19 and BCMA, a CAR binding GCC and tMUC1, and a CAR binding CD19 and GCC. If a CAR has more than two antigen binding domains, these antigen binding domains can be in series on the same CAR molecule. In embodiments, the one or more antigen binding domains are for expanding and/or maintaining modified cells, such as a CAR T cell, or for killing a tumor cell, such as a solid tumor. In embodiments, the one or more antigen binding domains for expanding and/or maintaining modified cells bind one or more antigens, for example, a cell surface molecule or marker, on the surface of a WBC.

In embodiments, the WBC is at least one of GMP (granulocyte macrophage precursor), MDP (monocyte-macrophage/dendritic cell precursors), cMoP (common monocyte precursor), basophil, eosinophil, neutrophil, SatM (Segerate-nucleus-containing atypical monocyte), macrophage, monocyte, CDP (common dendritic cell precursor), cDC (conventional DC), pDC (plasmacytoid DC), CLP (common lymphocyte precursor), B cell, ILC (Innate Lymphocyte), NK cell, megakaryocyte, myeloblast, pro-myelocyte, myelocyte, meta-myelocyte, band cells, lymphoblast, prolymphocyte, monoblast, megakaryoblast, promegakaryocyte, megakaryocyte, platelets, or MSDC (Myeloid-derived suppressor cell). In embodiments, the WBC is a granulocyte, monocyte, and or lymphocyte. In embodiments, the WBC is a lymphocyte, for example, a B cell. In embodiments, the WBC is a B cell. In embodiments, the cell surface molecule of a B cell includes CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the cell surface molecule of the B cell is CD19, CD20, CD22, or BCMA. In embodiments, the cell surface molecule of the B cell is CD19.

In embodiments, the CAR molecules described herein comprise one or more complementarity-determining regions (CDRs) for binding an antigen of interest. CDRs are part of the variable domains in immunoglobulins and T cell receptors (TCRs) for binding a specific antigen. There are three CDRs for each variable domain. Since there is a variable heavy domain and a variable light domain, there are six CDRs for binding an antigen. Further since an antibody has two heavy chains and two light chains, an antibody has twelve CDRs altogether for binding antigens. In embodiments, the CAR molecules comprise one or more CDRs for binding one or more antigens described herein.

The cells described herein, including modified cells such as CAR cells including CAR T cells, and modified T cells, can be derived from stem cells. Stem cells may be adult stem cells, embryonic stem cells, more particularly non-human stem cells, cord blood stem cells, progenitor cells, bone marrow stem cells, induced pluripotent stem cells, totipotent stem cells, or hematopoietic stem cells. A modified cell may also be a dendritic cell, an NK cell, a B cell, or a T cell selected from the group consisting of inflammatory T lymphocytes, cytotoxic T lymphocytes, regulatory T lymphocytes, or helper T lymphocytes. In embodiments, Modified cells may be derived from the group consisting of CD4+T lymphocytes and CD8+T lymphocytes. Prior to the expansion and genetic modification of the cells of the invention, a source of cells may be obtained from a subject through a variety of non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available and known to those skilled in the art may be used. In embodiments, modified cells may be derived from a healthy donor, from a patient diagnosed with cancer, or from a patient diagnosed with an infection. In embodiments, a modified cell is part of a mixed population of cells that present different phenotypic characteristics.

A population of cells refers to a group of two or more cells. The cells of the population could be the same, such that the population is a homogenous population of cells. The cells of the population could be different, such that the population is a mixed population or a heterogeneous population of cells. For example, a mixed population of cells could include modified cells comprising a first CAR and cells comprising a second CAR, wherein the first CAR and the second CAR bind different antigens.

The term “stem cell” refers to any of certain types of cell which have the capacity for self-renewal and the ability to differentiate into other kind(s) of a cell. For example, a stem cell gives rise either to two daughter stem cells (as occurs in vitro with embryonic stem cells in culture) or to one stem cell and a cell that undergoes differentiation (as occurs, e.g., in hematopoietic stem cells, which give rise to blood cells). Different categories of stem cells may be distinguished on the basis of their origin and/or on the extent of their capacity for differentiation into other types of cells. For example, stem cells may include embryonic stem (ES) cells (i.e., pluripotent stem cells), somatic stem cells, induced pluripotent stem cells, and any other types of stem cells.

The pluripotent embryonic stem cells are found in the inner cell mass of a blastocyst and have an innate capacity for differentiation. For example, pluripotent embryonic stem cells have the potential to form any type of cell in the body. When grown in vitro for long periods of time, ES cells maintain pluripotency as progeny cells retain the potential for multilineage differentiation.

Somatic stem cells can include fetal stem cells (from the fetus) and adult stem cells (found in various tissues, such as bone marrow). These cells have been regarded as having a capacity for differentiation that is lower than that of the pluripotent ES cells—with the capacity of fetal stem cells being greater than that of adult stem cells. Somatic stem cells apparently differentiate into only a limited number of types of cells and have been described as multipotent. The “tissue-specific” stem cells normally give rise to only one type of cell. For example, embryonic stem cells may be differentiated into blood stem cells (e.g., Hematopoietic stem cells (HSCs)), which may be further differentiated into various blood cells (e.g., red blood cells, platelets, white blood cells, etc.).

Induced pluripotent stem cells (i.e., iPS cells or iPSCs) may include a type of pluripotent stem cell artificially derived from a non-pluripotent cell (e.g., an adult somatic cell) by inducing an expression of specific genes. Induced pluripotent stem cells are similar to natural pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be obtained from adult stomach, liver, skin, and blood cells.

In embodiments, the antigen binding domain for killing a tumor binds an antigen on the surface of a tumor, for example, a tumor antigen or tumor marker. Tumor antigens are proteins that are produced by tumor cells that elicit an immune response, particularly T cell mediated immune responses. Tumor antigens are well known in the art and include, for example, tumor associated MUC1 (tMUC1), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, CD19, and mesothelin. For example, when the tumor antigen is CD19, the CAR thereof can be referred to as CD19 CAR or 19CAR, which is a CAR molecule that includes an antigen binding domain that binds CD19.

In embodiments, the extracellular antigen binding domain of a CAR includes at least one scFv or at least a single domain antibody. As an example, there can be two scFvs on a CAR. The scFv includes a light chain variable (VL) region and a heavy chain variable (VH) region of a target antigen-specific monoclonal antibody joined by a flexible linker. Single chain variable region fragments can be made by linking light and/or heavy chain variable regions by using a short linking peptide (Bird et al., Science 242:423-426, 1988). An example of a linking peptide is the GS linker having the amino acid sequence (GGGGS)₃ (SEQ ID NO: 1), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. The single chain variants can be produced either recombinantly or synthetically. For the synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect, or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The cytoplasmic domain of the CAR molecules described herein includes one or more co-stimulatory domains and one or more signaling domains. The co-stimulatory and signaling domains function to transmit the signal and activate molecules, such as T cells, in response to antigen binding. The one or more co-stimulatory domains are derived from stimulatory molecules and/or co-stimulatory molecules, and the signaling domain is derived from a primary signaling domain, such as the CD3 zeta domain. In embodiments, the signaling domain further includes one or more functional signaling domains derived from a co-stimulatory molecule. In embodiments, the co-stimulatory molecules are cell surface molecules (other than antigens receptors or their ligands) that are required for activating a cellular response to an antigen.

In embodiments, the co-stimulatory domain includes the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In embodiments, the signaling domain includes a CD3 zeta domain derived from a T cell receptor.

The CAR molecules described herein also include a transmembrane domain. The incorporation of a transmembrane domain in the CAR molecules stabilizes the molecule. In embodiments, the transmembrane domain of the CAR molecules is the transmembrane domain of a CD28 or 4-1BB molecule.

Between the extracellular domain and the transmembrane domain of the CAR, there may be incorporated a spacer domain. As used herein, the term “spacer domain” generally means any oligo- or polypeptide that functions to link the transmembrane domain to the extracellular domain and/or the cytoplasmic domain on the polypeptide chain. A spacer domain may include up to 300 amino acids, preferably 10 to 100 amino acids, and most preferably 25 to 50 amino acids.

The present disclosure describes a method of activating and/or expanding modified cells in vivo or in vitro, the method comprising obtaining modified cells comprising a binding molecule that binds a solid tumor antigen; contacting the modified cells with a population of cells comprising an antigen of a WBC and a PBM, the PBM comprising a first binding domain binding a T cell and a second binding domain binding the antigen of a WBC; and allowing the modified cells to expand and/or to be activated.

The present disclosure describes a method of enhancing activation and/or expansion of modified cells in vivo or in vitro, the method comprising obtaining modified cells comprising a binding molecule that binds a solid tumor antigen; contacting the modified cells with a population of cells comprising an antigen of a WBC and a PBM, the PBM comprising a first binding domain binding a T cell and a second binding domain binding the antigen of the WBC; and allowing the modified cells to expand and/or to be activated. In embodiments, the level of expansion and/or activation of the modified cells is higher than the level of expansion and/or activation of the modified cells that are contacted with the population of cells in the absence of the PBM. In embodiments, the modified cells are treated using CD3/CD28 agonists before contact with the PBM.

The present disclosure describes a method of causing or enhancing T cell response of a population of T cells in vivo or in vitro, the method comprising obtaining T cells comprising a binding molecule that binds a solid tumor antigen; contacting the T cells with a population of cells comprising an antigen of a WBC and a PBM, thereby causing or enhancing the T cell response of the T cells, the PBM comprising a first binding domain binding a T cell and a second binding domain binding the antigen of the WBC. In embodiments, the T cell response may be measured based on a level of activation and/or expansion of the T cells. In embodiments, the T cells are treated using CD3/CD28 agonists before being contacted with the PBM.

In embodiments, the activation is measured based on a level of expression of CD69, CD25, or CD137 on the modified cells. In embodiments, the expansion is measured based on the numbers of cells in the population or copy numbers of DNA encoding the CAR.

The present disclosure describes a method of treating a subject having cancer or enhancing the treatment thereof, the method comprising administering an effective amount of a composition comprising modified cells to the subject having a form of cancer associated with or expressing a solid tumor antigen; administering an effective amount of PBM comprising a first binding domain binding a T cell and a second binding domain binding the antigen of a WBC. In embodiments, the T cells are treated using CD3/CD28 agonists before being contacted with the PBM.

The present disclosure describes a kit comprising an effective amount of modified cells and an effective amount of PBM. For example, the PBM is a bispecific antibody or a BITE® comprising a first binding domain binding CD3 and a second binding domain binding CD19, CD20, CD22, or BCMA, and the modified cells are CAR T cells. Examples of PBMs are listed in Table 7.

The present disclosure describes a kit comprising an effective amount of first PBMs targeting a WBC antigen and an effective amount of second PBM targeting a solid tumor antigen. In embodiments, the first PBM is a BITE® comprising a first binding domain binding CD3 and a second binding domain binding CD19, CD22, CD20, BCMA, CD5, CD7, CD2, CD16, CD56, CD30, CD14, CD68, CD11b, CD18, CD169, CD1c, CD33, CD38, CD138, or CD13. In embodiments, the second PBM is a BITE® comprising a first binding domain binding CD3 and a second binding domain binding tMUC1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, B7-H3, CLDN18.2, MAGE A4, MSLN, CD205, or EGFR. Examples of first and second PBMs are listed in Table 7. In embodiments, the kit may be used with a combination of one or more cytokines, which may be administered directly to a subject having cancer, in a form of vaccines (mRNA nanoparticle), and/or in a form of cells (APCs) that express the one or more cytokines. Examples of cytokines include IL-1P, IL-2, IL-4, IL-5, IL-6, IL-7, IL-8, IL-10, IL-12, IL-13, IL-15, IL-17, IL-1Ra, IL-2R, IFNγ, MIP-In, MIP-IP, MCP-1, TNFα, GM-CSF, GCSF, CXCL9, CXCL10, CXCR factors, VEGF, RANTES, EOTAXIN, EGF, HGF, FGF-P, CD40, CD40L, and ferritin. In embodiments, the one or more cytokines include IL-2, IL-12, IL-6, IFN, or IL-7, GCSF, GM-CSF, and/or CXCL9.

The present disclosure describes a pharmaceutical composition comprising an effective amount of modified cells and an effective amount of PBM. For example, the PBM is a BITE® comprising a first binding domain binding CD3 and a second binding domain binding CD19, CD20, CD22, or BCMA.

In embodiments, the solid tumor antigen comprises tMUC1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, B7-H3, CLDN18.2, MAGE A4, MSLN, CD205, or EGFR.

In embodiments, examples of WBCs comprise a granulocyte, a monocyte, or a lymphocyte. In embodiments, the modified cells, such as CAR cells, are T cells, NK cells, macrophages, or dendritic cells. For example, the modified cells are T cells. In embodiments, an antigen of the WBC comprises CD19, CD22, CD20, BCMA, CD5, CD7, CD2, CD16, CD56, CD30, CD14, CD68, CD11b, CD18, CD169, CD1c, CD33, CD38, CD138, CD205, CD79a, CD79b, or CD13.

In embodiments, the binding molecule is a chimeric antigen receptor (CAR) or a TCR. In embodiments, the CAR comprises an antigen binding domain, a transmembrane domain, a co-stimulatory domain, and a CD3 zeta domain, and the co-stimulatory domain comprises the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof. In embodiments, the TCR binds CEA, gp100, MART-1, p53, MAGE-A3, or NY-ESO-1.

In embodiments, the CAR T or modified cells comprise an exogenous polynucleotide encoding a therapeutic agent comprising IL-1P, IL-2, IL-4, IL-5, IL-6, IL-7, IL-8, IL-10, IL-12, IL-13, IL-15, IL-17, IL-1Ra, IL-2R, IFN-γ, MIP-In, MIP-IP, MCP-1, TNFα, GM-CSF, GCSF, CXCL9, CXCL10, CXCR factors, VEGF, RANTES, EOTAXIN, EGF, HGF, FGF-P, CD40, CD40L, or ferritin.

In embodiments, the CAR T or modified cells comprise a dominant negative form of PD-1, cytotoxic T lymphocyte antigen-4 (CTLA-4), B- and T-lymphocyte attenuator (BTLA), T-cell immunoglobulin mucin-3 (TIM-3), lymphocyte-activation protein 3 (LAG-3), T-cell immunoreceptor with Ig and ITIM domains (TIGIT), leukocyte-associated immunoglobulin-like receptor 1 (LAIRI), natural killer cell receptor 2B4 (2B4), or CD160.

In embodiments, the modified cells are derived from tumor-infiltrating lymphocytes (TILs). In embodiments, a T cell clone that expresses a TCR with a high affinity for the target antigen may be isolated. TILs or peripheral blood mononuclear cells (PBMCs) can be cultured in the presence of antigen-presenting cells (APCs) pulsed with a peptide representing an epitope known to elicit a dominant T cell response when presented in the context of a defined HLA allele. High-affinity clones may be then selected on the basis of MHC-peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCRβ gene sequences are then used to generate an expression construct that ideally promotes stable, high-level expression of both TCR chains in human T cells. The transduction vehicle, for example, a gammaretrovirus or lentivirus, can then be generated and tested for functionality (antigen specificity and functional avidity) and used to produce a clinical lot of the vector. An aliquot of the final product can then be used to transduce the target T cell population (generally purified from patient PBMCs), which is expanded before infusion into the patient.

Various methods may be implemented to obtain genes encoding tumor-reactive TCR. More information is provided in Kershaw et al., Clin Transl Immunology. 2014 May; 3(5): e16. In embodiments, specific TCR can be derived from spontaneously occurring tumor-specific T cells in patients. Antigens included in this category include the melanocyte differentiation antigens MART-1 and gp100, as well as the MAGE antigens and NY-ESO-1, with expression in a broader range of cancers. TCRs specific for viral-associated malignancies can also be isolated, as long as viral proteins are expressed by transformed cells. Malignancies in this category include liver and cervical cancer, those associated with hepatitis and papilloma viruses, and Epstein-Barr virus-associated malignancies. In embodiments, target antigens of the TCR include CEA (e.g., for colorectal cancer), gp100, MART-1, p53 (e.g., for melanoma), MAGE-A3 (e.g., melanoma, esophageal and synovial sarcoma), and NY-ESO-1 (e.g., for melanoma and sarcoma as well as multiple myelomas).

In embodiments, preparation and transfusion of tumor infiltrating lymphocytes (TIL) may be implemented in the following manner. For example, tumor tissue coming from surgical or biopsy specimens can be obtained under aseptic conditions and transported to the cell culture chamber in an ice box. Necrotic tissue and adipose tissue can be removed. The tumor tissue can be cut into small pieces of about 1-3 cubic millimeters. Collagenase, hyaluronidase, and DNA enzyme can be added and digested overnight at 4° C. Filtering with 0.2 μm filter, cells can be separated and collected by lymphocyte separation fluid, under 1500 rpm for 5 min. Expanding the cells in a culture medium comprising PHA, 2-mercaptoethanol, and a CD3 monoclonal antibody, and a small dose of IL-2 (10-20 IU/ml) may be added to induce activation and proliferation. The cell density may be carefully measured and maintained within the range of 0.5-2×10⁶/ml for 7-14 days at a temperature of 37° C. with 5% CO₂. TIL positive cells having the ability to kill homologous cancer cells can be screened out by co-culture. The TIL positive cells can be amplified in a serum-free medium containing a high dose of IL-2 (5000-6000 IU/ml) until greater than 1×10¹¹ TILs can be obtained. To administer TILs, they are first collected in saline using continuous-flow centrifugation and then filtered through a platelet-administration set into a volume of 200-300 mL containing 5% albumin and 450000 IU of IL-2. The TILs can be infused into patients through a central venous catheter over a period of 30-60 minutes. In embodiments, TILs can be infused in two to four separate bags, and the individual infusions can be separated by several hours.

In embodiments, the increase in T cell response is based on the increase in the number of copies of CAR(s) and/or the amount of cytokine released (e.g., IL-6 and IFNγ. In embodiments, the T cell response comprises cytokine releases, cell expansion, and/or activation levels. In embodiments, the modified cells comprise a polynucleotide encoding IL-6 or IFNγ, or a combination thereof. In embodiments, the modified cells comprise a polynucleotide encoding IL-12. In embodiments, the polynucleotide comprises a polynucleotide encoding NFAT and/or VHL.

In embodiments, the modified cells comprise a bispecific CAR. In embodiments, the bispecific CAR can have two different scFv molecules joined together by linkers. Examples of the bispecific CAR are provided in Table 2.

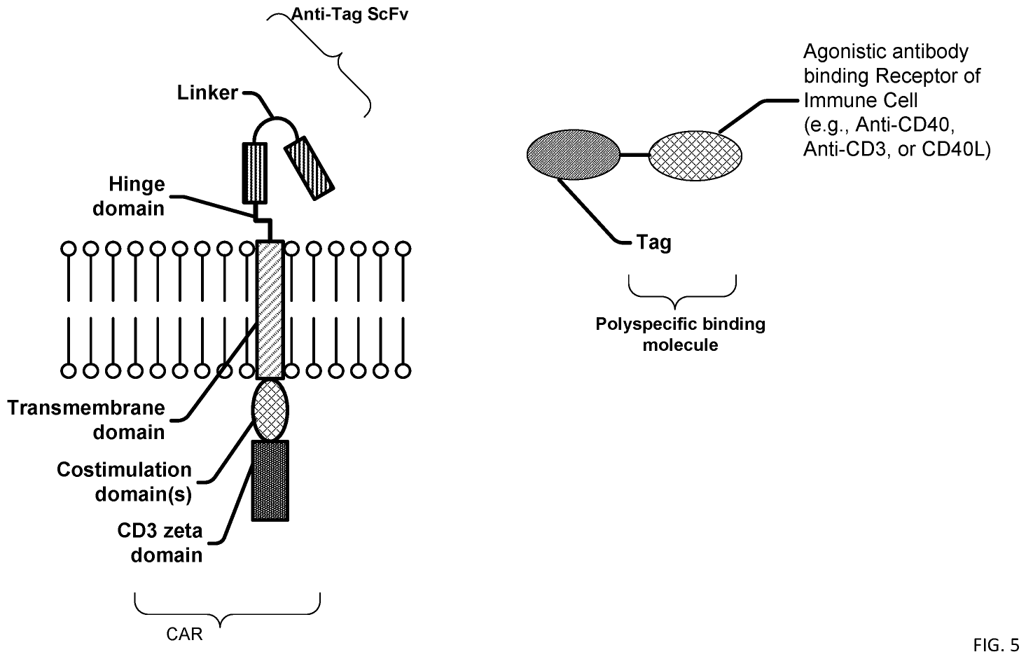

An example of a bispecific CAR is shown in FIG. 5. As shown in FIG. 5, a bispecific CAR (or tandem CAR (tanCAR)) including two binding domains: scFv1 and scFv2. In embodiments, scFv1 binds an antigen of a white blood cell (e.g., CD19), and scFv2 binds a solid tumor antigen (e.g., tMUC1). In embodiments, scFv1 binds a solid tumor antigen, and scFv2 binds another solid tumor antigen (e.g., tMUC1 and CLDN 18.2). Claudin18.2 (CLDN 18.2) is a stomach-specific isoform of Claudin-18. CLDN 18.2 is highly expressed in gastric and pancreatic adenocarcinoma. In embodiments, scFv1 binds an antigen expressed on tumor cells but not on normal tissues (e.g., tMUC1); scFv2 binds an antigen expressed on nonessential tissues associated with solid tumor, and the killing of normal cells of the tissue does not cause a life-threatening event (e.g., complications) to the subject (e.g., TSHR, GUCY2C). Examples of the nonessential tissues include organs such as prostate, breast, or melanocyte. In embodiments, scFv1 and scFv2 bind to different antigens that are expressed on the same nonessential tissue (e.g., ACPP and SLC45A3 for Prostate cancer, and SIGLEC15 and UPK2 for Urothelial cancer). Examples of bispecific CARs and their components are shown in Table 2.

TABLE 2

| Variable | | Variable | | Variable | | Variable |
|---|---|---|---|---|---|---|
| domain 1 | Linker 1 | domain 3 | Linker 2 | domain 5 | Linker 3 | domain 7 |
| Anti-TSHR- | 3*GGGGS | Anti-TSHR- | 4*GGGGS | humanized- | 3*GGGGS | humanized- |
| VL | (SEQ ID | VH | (SEQ ID | anti CD19- | linker | anti CD19-VL |
| | NO: 1) | | NO: 23) | VH | | |
| | linker | | bispecific | | | |
| | | | CAR linker | | | |
| Anti-TSHR- | 3*GGGGS | Anti-TSHR- | 4*GGGGS | humanized- | 3*GGGGS | humanized- |
| VH | linker | VL | bispecific | anti CD19- | linker | anti CD19-VH |
| | | | CAR linker | VL | | |
| Tumor | 3*GGGGS | Tumor | 4*GGGGS | anti CD19- | 3*GGGGS | anti CD19-VH |
| associated | linker | associated | bispecific | VL | linker | |
| MUC1 | | MUC1 | CAR linker | | | |
| scFv-1 or 2 | | scFv-1 or 2 | | | | |
| VL | | VH | | | | |
| Tumor | 3*GGGGS | Tumor | 4*GGGGS | anti CD19- | 3*GGGGS | anti CD19-VL |
| associated | linker | associated | bispecific | VH | linker | |
| MUC1 | | MUC1 | CAR linker | | | |
| scFv-1 or 2 | | scFv-1 or 2 | | | | |
| VH | | VL | | | | |
| humanized- | 3*GGGGS | humanized- | 4*GGGGS | Tumor | 3*GGGGS | Tumor |
| anti CD19- | linker | anti CD19- | bispecific | associated | linker | associated |
| VH | | VL | CAR linker | MUC1 | | MUC1 scFv-1 |
| | | | | scFv-1 or 2 | | or 2 VH |
| | | | | VL | | |
| Tumor | 3*GGGGS | Tumor | 4*GGGGS | Anti-TSHR- | 3*GGGGS | Anti-TSHR-VH |
| associated | linker | associated | bispecific | VL | linker | |
| MUC1 | | MUC1 | CAR linker | | | |
| scFv-1 or 2 | | scFv-1 or 2 | | | | |
| VL | | VH | | | | |
| Anti-TSHR- | 3*GGGGS | Anti-TSHR- | 4*GGGGS | Tumor | 3*GGGGS | Tumor |
| VL | linker | VH | bispecific | associated | linker | associated |
| | | | CAR linker | MUC1 | | MUC1 scFv-1 |
| | | | | scFv-1 or 2 | | or 2 VH |
| | | | | VL | | |
| Tumor | 3*GGGGS | Tumor | 4*GGGGS | Anti- | 3*GGGGS | Anti-GUCY2C- |
| associated | linker | associated | bispecific | GUCY2C- | linker | VL or VH |
| MUC1 | | MUC1 | CAR linker | VH or VL | | |
| scFv-1 or 2 | | scFv-1 or 2 | | | | |
| VL | | VH | | | | |
| Anti- | 3*GGGGS | Anti- | 4*GGGGS | Tumor | 3*GGGGS | Tumor |
| GUCY2C- | linker | GUCY2C- | bispecific | associated | linker | associated |
| VH or VL | | VL or VH | CAR linker | MUC1 | | MUC1 scFv-1 |
| | | | | scFv-1 or 2 | | or 2 VH |
| | | | | VL | | |
| Tumor | 3*GGGGS | Tumor | 4*GGGGS | Anti-ACPP- | 3*GGGGS | Anti-ACPP- |
| associated | linker | associated | bispecific | VH or VL | linker | VL or VH |
| MUC1 | | MUC1 | CAR linker | | | |
| scFv-1 or 2 | | scFv-1 or 2 | | | | |
| VL | | VH | | | | |
| Anti-ACPP- | 3*GGGGS | Anti-ACPP- | 4*GGGGS | Tumor | 3*GGGGS | Tumor |
| VH or VL | linker | VL or VH | bispecific | associated | linker | associated |
| | | | CAR linker | MUC1 | | MUC1 scFv-1 |
| | | | | scFv-1 or 2 | | or 2 VH |
| | | | | VL | | |
| Tumor | 3*GGGGS | Tumor | 4*GGGGS | Anti- | 3*GGGGS | Anti- |
| associated | linker | associated | bispecific | CLDN18.2- | linker | CLDN18.2-VL |
| MUC1 | | MUC1 | CAR linker | VH or VL | | or VH |
| scFv-1 or 2 | | scFv-1 or 2 | | | | |
| VL | | VH | | | | |
| Anti- | 3*GGGGS | Anti- | 4*GGGGS | Tumor | 3*GGGGS | Tumor |
| CLDN18.2- | linker | CLDN18.2- | bispecific | associated | linker | associated |
| VH or VL | | VL or VH | CAR linker | MUC1 | | MUC1 scFv-1 |
| | | | | scFv-1 or 2 | | or 2 VH |
| | | | | VL | | |
| Tumor | 3*GGGGS | Tumor | 4*GGGGS | Anti-UPK2- | 3*GGGGS | Anti-UPK2- |
| associated | linker | associated | bispecific | VH or VL | linker | VL or VH |
| MUC1 | | MUC1 | CAR linker | | | |
| scFv-1 or 2 | | scFv-1 or 2 | | | | |
| VL | | VH | | | | |
| Anti-UPK2- | 3*GGGGS | Anti-UPK2- | 4*GGGGS | Tumor | 3*GGGGS | Tumor |
| VH or VL | linker | VL or VH | bispecific | associated | linker | associated |
| | | | CAR linker | MUC1 | | MUC1 scFv-1 |
| | | | | scFv-1 or 2 | | or 2 VH |
| | | | | VL | | |
| Tumor | 3*GGGGS | Tumor | 4*GGGGS | Anti- | 3*GGGGS | Anti- |
| associated | linker | associated | bispecific | SIGLEC15- | linker | SIGLEC15-VL |
| MUC1 | | MUC1 | CAR linker | VH or VL | | or VH |
| scFv-1 or 2 | | scFv-1 or 2 | | | | |
| VL | | VH | | | | |
| Anti- | 3*GGGGS | Anti- | 4*GGGGS | Tumor | 3*GGGGS | Tumor |
| SIGLEC15- | linker | SIGLEC15- | bispecific | associated | linker | associated |
| VH or VL | | VL or VH | CAR linker | MUC1 | | MUC1 scFv-1 |
| | | | | scFv-1 or 2 | | or 2 VH |
| | | | | VL | | |

3*(GGGGS) is (GGGGS)₃ and 4*(GGGGS) is (GGGGS)₄.

Lymphocyte or T cell response in a subject refers to cell-mediated immunity associated with a helper, killer, regulatory, and other types of T cells. For example, T cell response may include activities such as assisting other WBCs in immunologic processes and identifying and destroying virus-infected cells and tumor cells. T cell response in the subject can be measured via various indicators such as the number of virus-infected cells and/or tumor cells that T cells kill, the amount of cytokines (e.g., IL-6 and IFNγ) that T cells release in vivo and/or in co-culturing with virus-infected cells and/or tumor cells, a level of proliferation of T cells in the subject, a phenotype change of T cells, for example, changes to memory T cells, and the level of longevity or lifetime of T cells in the subject.

In embodiments, the method of enhancing T cell response described herein can effectively treat a subject in need thereof, for example, a subject diagnosed with a tumor. The term tumor refers to a mass, which can be a collection of fluid, such as blood, or a solid mass. A tumor can be malignant (cancerous) or benign. Examples of blood cancers include chronic lymphocytic leukemia, acute myeloid leukemia, acute lymphoblastic leukemia, and multiple myeloma.

Solid tumors usually do not contain cysts or liquid areas. The major types of malignant solid tumors include sarcomas and carcinomas. Sarcomas are tumors that develop in soft tissue cells called mesenchymal cells, which can be found in blood vessels, bone, fat tissues, ligament lymph vessels, nerves, cartilage, muscle, ligaments, or tendon, while carcinomas are tumors that form in epithelial cells, which are found in the skin and mucous membranes. The most common types of sarcomas include undifferentiated pleomorphic sarcoma, which involves soft tissue and bone cells; leiomyosarcoma, which involves smooth muscle cells that line blood vessels, gastrointestinal tract, and uterus; osteosarcoma, which involves bone cells; and liposarcoma which involves fat cells. Some examples of sarcomas include Ewing sarcoma, Rhabdomyosarcoma, chondosarcoma, mesothelioma, fibrosarcoma, fibrosarcoma, and glioma.

The five most common carcinomas include adrenocarcinoma, which involves organs that produce fluids or mucous, such as the breasts and prostate; basal cell carcinoma, which involves cells of the outer-most layer of the skin, for example, skin cancer; squamous cell carcinoma, which involves the basal cells of the skin; and transitional cell carcinoma which affects transitional cells in the urinary tract which includes the bladder, kidneys, and ureter. Examples of carcinomas include cancers of the thyroid, breast, prostate, lung, intestine, skin, pancreas, liver, kidneys, and bladder, and cholangiocarcinoma.

The methods described herein can be used to treat a subject diagnosed with cancer. Cancer can be a blood cancer or a solid tumor, such as a sarcoma or carcinoma. The method of treating includes administering an effective amount of a mixed population of T cells described herein comprising a first antigen binding domain and/or a second antigen binding domain to the subject to provide a T-cell response, wherein the first antigen binding domain binds a cell surface molecule of a WBC, and the second antigen binding domain binds an antigen different from the cell surface molecule of the WBC. In embodiments, enhancing the T cell response in the subject includes selectively enhancing the proliferation of the mixed population of T cells comprising the first antigen binding domain and the second antigen binding domain in vivo.

Conventional bispecific and/or trispecific antibodies have limitations, as they can stimulate T cells but have poor efficacy in solid tumors. Embodiments herein change structures of the conventional bispecific and/or trispecific antibodies such that they can interact with more cells. Additional information on bispecific and trispecific antibodies can be found at Runcie et al. Molecular Medicine (2018) 24:50, which is incorporated by its reference. For example, a first group of bispecific and/or trispecific antibodies may target a WBC (e.g., B cell) and CD3, and a second group of bispecific and/or trispecific antibodies may target solid tumor tissue via a solid tumor antigen and CD3. In the case of trispecific antibodies, the trispecific antibodies may further bind to a factor. Examples of the factors include IL1-a, IL1-β, IL2, IL6, IL8, IL12, IL7, IL15, IL18, IL21, and IL27.

In embodiments, the bispecific and/or trispecific binding molecules (e.g., antibodies or TCR-antibody) may stimulate innate immunity. The ipsilateral co-stimulated end can be selected from the activated antibody/ligand of the previously provided receptor list.

In embodiments, the bispecific and/or trispecific binding molecules may activate monocyte, macrophage, neutrophil, dendritic cell, and/or T cell and bind a targeted tumor. In embodiments, the bispecific and/or trispecific binding molecules may further comprise cytokines. In embodiments, the bispecific and/or trispecific binding molecules may be combined with CAR T/TCR/TIL/NK cell based therapy. Thus, the bispecific and/or trispecific binding molecules may activate natural immunity (monocyte, macrophage, neutrophil, dendritic cell, and the like) and coupling the natural immunity with the killing of tumor cells.

In embodiments, the bispecific and/or trispecific binding molecules may include an anti-tag domain and an anti-CD40 domain. The anti-tag domain is an antibody or ligand that binds a tag, an artificial antigen, for safety purposes. For example, an agent comprising the tag may be administered to a subject to trigger activities of the bispecific and/or trispecific binding molecules. If the administration of the agent is discontinued, the activities of the bispecific and/or trispecific binding molecules will be terminated. The bispecific and/or trispecific binding molecules may be used in combination with modified cells (e.g., T cell and NK cell) comprising a CAR having the tag.

Moreover, the present disclosure describes a combination of GCSF and TIL technology. According to the experiment of CoupledCAR®, it is known that T cells can be expanded in non-tumor environments such as peripheral blood. Additional information regarding CoupledCAR® can be found in PCT Publication WO 2020146743, which is incorporated herein by reference in its entirety. Under conventional technology, TIL can be expanded in vitro and then reinfused to treat tumors. Embodiments herein may drive TILs out of place using a mobilizer like GCSF and then give CoupledCAR® (or anti-CD19 & CD3 BiTEs) and/or cytokine(s). Thus, the expansion of TILs in the human body, in a non-tumor environment, to achieve anti-cancer.

The present disclosure describes an in vitro method for preparing modified cells. The method may include obtaining a sample of cells from a subject. For example, the sample may include T cells or T cell progenitors. The method may further include transfecting the sample of cells with a DNA encoding at least a CAR and culturing the population of CAR cells ex vivo in a medium that selectively enhances the proliferation of CAR-expressing T cells.

In embodiments, the sample is a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T cells.

The present disclosure describes an isolated nucleic acid sequence encoding a CAR, wherein the CAR comprises an extracellular domain, a transmembrane domain, and an intracellular domain, the extracellular domain comprises an extracellular domain of a receptor of a homing molecule, and the intracellular domain comprises an intracellular domain of a T cell activation molecule. Embodiments relate to a population of CAR cells comprising the CAR. Embodiments relate to a pharmaceutical composition comprising the population of the CAR cells. Embodiments relate to a method of causing or enhancing a T cell response in a subject in need thereof and/or treating a tumor in the subject, wherein the method comprises administering an effective amount of the pharmaceutical composition to the subject. In embodiments, the transmembrane domain of the CAR comprises a transmembrane domain of a T cell receptor. As used herein, causing a T cell response includes stimulating a T cell response.

The present disclosure describes pharmaceutical compositions comprising cells including modified cells, such as CAR T cells. Pharmaceutical compositions described herein may be administered in a manner appropriate to the disease to be treated such as cancer. The quantity and frequency of administration will be determined by such factors as the condition of the patient, and the type and severity of the patient's disease, although appropriate dosages may be determined by clinical trials.

The term “pharmaceutically acceptable” means approved by a regulatory agency of the U.S. Federal or a state government or the EMA (European Medicines Agency) or listed in the U.S. Pharmacopeia (United States Pharmacopeia-33/National Formulary-28 Reissue, published by the United States Pharmacopeial Convention, Inc., Rockville Md., publication date: April 2010) or other generally recognized pharmacopeia for use in animals, and more particularly in humans.

The term “carrier” refers to a diluent, adjuvant {e.g., Freund's adjuvant (complete and incomplete)), excipient, or vehicle with which the therapeutic is administered. Pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. For the use of (further) excipients and their use see also “Handbook of Pharmaceutical Excipients”, fifth edition, R. C. Rowe, P. J. Seskey and S. C. Owen, Pharmaceutical Press, London, Chicago.

A homing receptor refers to a receptor that causes a cell including the receptor, to home to a target including a particular anatomical zone, a particular tissue, a particular type of cells, e.g., B cell zone of the lymph nodes, skin, gastrointestinal tract, or tumor cell. Examples of homing receptor include CCR2, CCR4, CXCR3, CCR6, ICAM3, CCR7, LFA-3 CCR1, CCR3, and CCR5. In embodiments, the cell may be a lymphocyte (e.g., NK cell or T cell). For example, a T cell homing receptor may activate a T cell to home to the target (e.g., tumor cells). A tissue specific homing receptor may activate the cell to home to a particular tissue. In embodiments, the homing receptor is a chemotactic receptor (e.g., CXCR4, VEGFR2, and CCR 7). More examples of T cell homing receptors may be found in Sackstein et al., Laboratory Investigation (2017) 97, 669-697, which is incorporated by reference in its entirety.

A T cell activation molecule refers to a molecule that mediates a T cell response, including at least one of proliferation, activation, differentiation, and other cellular responses. Examples of T cell activation molecules include CD27, CD28, 4-IBB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, and B7-H3.

In embodiments, the extracellular domain of the CAR comprises or is an extracellular domain that can regulate T cell trafficking or can recruit Tregs to the tumor microenvironment related receptor proteins, such as CCR2, CCR4, CXCR3, CCR6, ICAM3, CCR7, LFA-3 CCR1, CCR3, or CCR5. The transmembrane domain of the CAR comprises or is a transmembrane region of T cell receptors, such as CD28, CD3 epsilon, CD45, CD4, CD5, CD8, CD9, CD16, CD22, CD33, CD37, CD64, CD80, CD86, CD134, CD137, or CD154. The intracellular domain of CAR comprises or is a molecule that activates T cells, such as CD27, CD28, 4-IBB (CD137), OX40, GITR, CD30, CD40, ICOS, BAFFR, HVEM, ICAM-1, lymphocyte function-associated antigen-1 (LFA-1), intracellular domain of CD2, CDS, CD7, CD287, LIGHT, NKG2C, NKG2D, SLAMF7, NKp80, NKp30, NKp44, NKp46, CD160, B7-H3 or a combination thereof. This CAR can enhance the efficacy of CAR T cells in immunotherapy by targeting T cells to the tumor microenvironment.

The present disclosure describes a modified cell comprising a chimeric antigen receptor (CAR), wherein the CAR comprises an extracellular domain, a transmembrane domain, and an intracellular domain, the extracellular domain comprising an extracellular domain of a receptor of a homing molecule, the intracellular domain comprising an intracellular domain of a T cell activation molecule.

In embodiments, the homing molecule comprises CCR2, CCR4, CXCR3, CCR6, ICAM3, CCR7, LFA-3, CCR1, CCR3, or CCR5. In embodiments, the T cell activation molecule comprises CD27, CD28, 4-IBB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, or a combination thereof. In embodiments, the transmembrane membrane comprises CD28, CD3 epsilon, CD45, CD4, CD5, CD8, CD9, CD16, CD22, CD33, CD37, CD64, CD80, CD86, CD134, CD137, or CD154.

In embodiments, the modified cell or cells comprise an additional CAR that binds an antigen. In embodiments, the intracellular domain of the additional CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

Under conventional technologies, the first generation of CAR was designed based on Signal 1 (CD3zeta), and the second generation was designed based on Signal 1 plus the co-stimulatory Signal (Signal 2). Embodiments described herein include Signal 1 for killing tumor cells, Signal 2 for activating T cells, and Signal 3 for expanding T cells without the CAR binding of an antigen.

In embodiments, the term “exogenous association motif” means any association motif that is recombinantly introduced into a domain, for example, an intracellular signaling domain such as a cytoplasmic domain of an interleukin receptor chain, a cytoplasmic co-stimulatory domain, or a CD3 intracellular signaling domain, but that does not exist natively in the domain or at a specific location in the domain. For example, an exogenous JAK-binding motif can be inserted into an intracellular signaling domain, such as, a cytoplasmic domain of an interleukin receptor chain. The “JAK-binding motif” used herein refers to a BOX-1 motif that allows for tyrosine kinase JAK association, for example, JAK1. The JAK-binding motif can be, for example, amino acid numbers 278 to 286 of NCBI RefSeq:NP_000869.1 (amino acids 13 to 21 SEQ ID NO: 5). In this instance, a “domain” means one region in a polypeptide, for example, which is folded into a particular structure independently of other regions and/or has a particular function. The domain can, for example, be the cytoplasmic portion of a molecule or a part thereof. As used herein, the “cytoplasmic domain” of a molecule can refer to the full cytoplasmic domain or a part thereof that induces an intracellular signal when activated.

The term “variant” refers to a molecule comprising a substitution, deletion, or addition of one or a few to a plurality of amino acids and includes particularly conservatively substituted molecules, provided that the variant substantially retains the same function as the original sequence. For example, IL receptor variants may comprise substitutions, deletions, or additions outside the JAK-binding motif and the STAT association motif. Thus, an IL receptor chain variant can comprise up to 50, up to 40, up to 30, up to 20, or up to 10 amino acid deletion and/or conservative substitutions in a region outside of the JAK-binding and STAT association motifs. Similarly, variants of other molecules can comprise up to 50, up to 40, up to 30, up to 20, or up to 10 amino acid deletion and/or conservative substitutions, in a region outside of a region identified specifically herein. As used herein, the phrase “wherein the intracellular segment comprises an endogenous or exogenous JAK-binding motif and a STATS association motif” includes an intracellular segment comprising more than one cytoplasmic domain, and the JAK binding motif and the STATS association motif may be in the same cytoplasmic domain or may be in separate cytoplasmic domains.

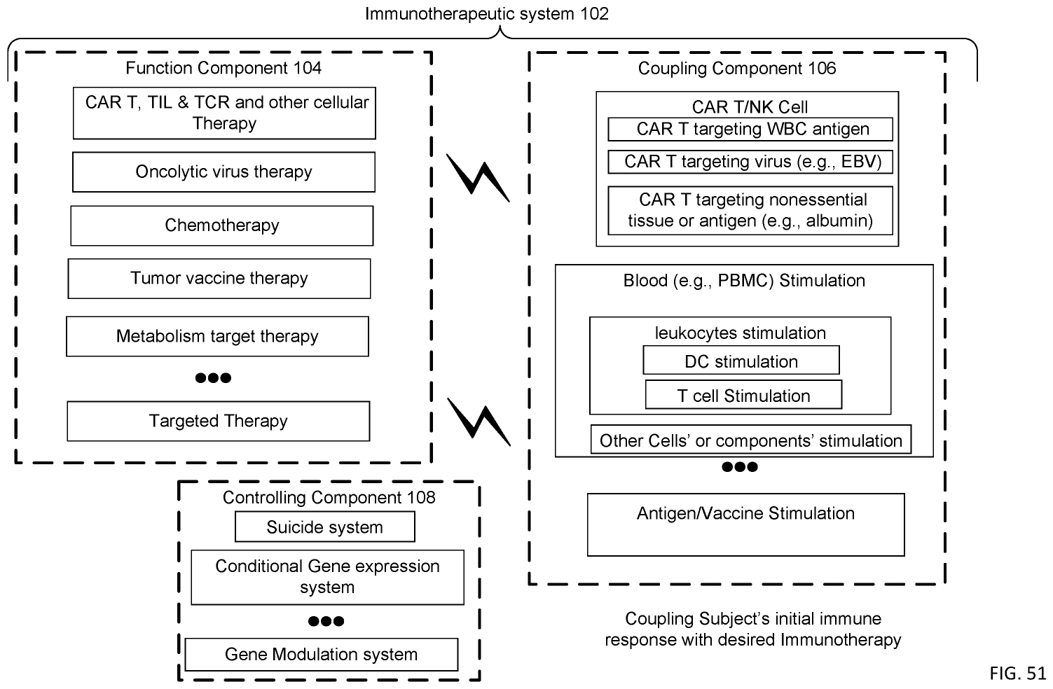

The present disclosure describes an immunotherapeutic system and its use for treating cancer of a subject. As shown in FIG. 51, the immunotherapeutic system 102 includes function component 104 configured to inhibit the growth of tumor cells, coupling component 106 configured to couple the subject's immune response with the inhibition of the growth of tumor cells and controlling component 108 configured to control the inhibition and/or coupling. In embodiments, the immunotherapeutic system 102 is a composition comprising one or more pharmaceutical compositions (e.g., antibodies and cells) suitable for treating cancer. For example, the immunotherapeutic system 102 may comprise CART cells or BiTEs targeting a solid tumor antigen (i.e., function component 104), CART cells or BiTEs targeting a WBC antigen (i.e., coupling component 106), and a suicide gene RQR8 incorporated into the CAR T cells (i.e., controlling component 108).

Examples of function component 104 include CAR T, TIL, and TCR and other cellular therapies, oncolytic virus therapy, chemotherapy, tumor vaccine therapy, metabolism target therapy, and targeted therapy. In embodiments, function component 104 includes at least one of the inhibitors that regulate immune metabolism (e.g., IDO inhibitors and adenosine inhibitors); the immunomodulators (e.g., IMiDs); the agonists against monocytes or dendritic cells (e.g., TLRs/STING); an oncolytic virus therapy; the tumor vaccines (e.g., DC vaccines); the tumor infiltrating T cells (e.g., Tils); the macrophage-reprogramming agents (e.g., CCR2-CCL2 inhibitor, CSF-1Rs inhibitor, PPAR-gamma agonist/inhibitor, and CD-40 agonist); the chemotherapy drugs (e.g., cyclophosphamide, fludarabine, and ibrutinib); the monoclonal antibody targeting drugs (e.g., anti-her2); or the targeted drugs for non-monoclonal antibodies (e.g., ALK inhibitors, EGF/VEGF inhibitors). Example targets of TCR therapy are listed in Table 6. In embodiments, function component 104 can be implemented by a BITE® molecule (e.g., TSHR-CD3). In an embodiment, a bispecific binding molecule, such as a bispecific antibody or a BITE® molecule, comprises a first and a second binding domain, wherein the first binding domain binds to a solid tumor antigen, and the second binding domain binds, for example, the T cell CD3 receptor complex or CD28. The second binding domain can also bind other T cell molecules such as 4-1BB, OX40, GTTR, ICOS, NKG20, etc.

Examples of coupling component 106 include immune response elicited by CAR T/NK cells, DC stimulation, T cell stimulation, and antigen/vaccine stimulation. The CAR T/NK cells include the modified cells described in the present disclosure. For example, the modified cell includes a CAR binding an antigen of WBC (e.g., CD19), an antigen of EBV, and/or albumin. T cell stimulation may be implemented by a bispecific binding molecule, such as a BITE® molecule (e.g., CD19-CD3). DC cell stimulation may be implemented by administering CAR T/NK cells to the subject or administering a small molecule, small peptide, vaccine, or antigen to lymphoid organs (e.g., lymph node) of the subject. In embodiments, a bispecific binding molecule, such as BITE® molecule, may comprise a first and a second binding domain, wherein the first binding domain binds to an antigen, and the second binding domain binds, for example, the T cell CD3 receptor complex or CD28. The second binding domain can bind other T cell molecules such as 4-1BB, OX40, GITR, ICOS, NKG20, etc. The first binding domain binds a WBC antigen (e.g., CD19 and BCMA). In embodiments, CART cells express the bispecific antibody, such as a BITE® molecule. In embodiments, CAR T cells and the bispecific antibody are administered to the subject sequentially or at the same time in separate administrations or in a single administration.

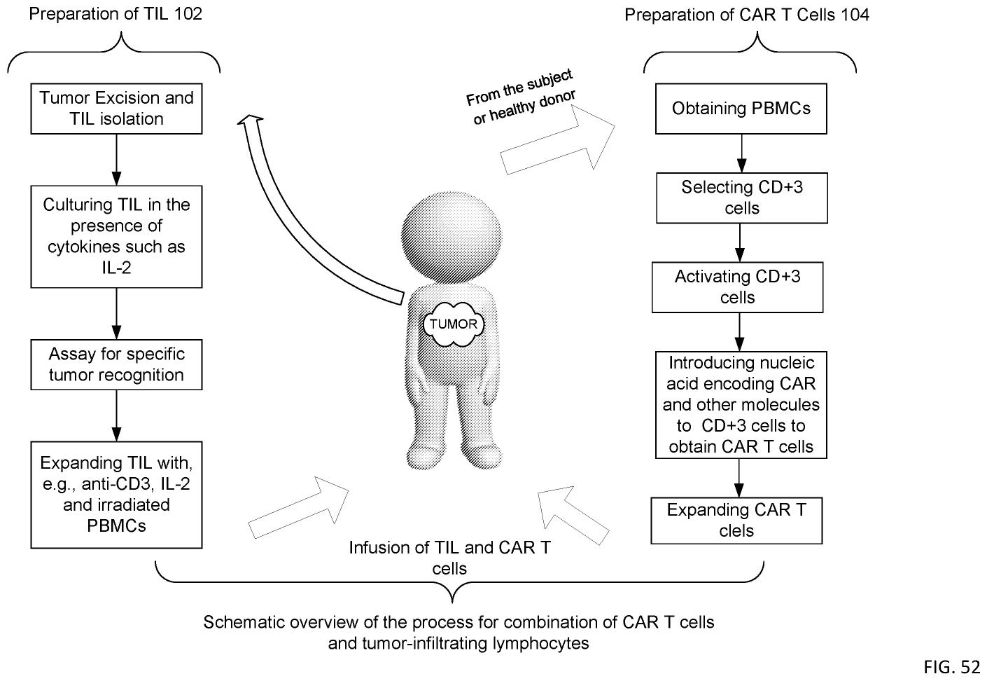

FIG. 52 shows a schematic overview of an exemplary process for the combination of CAR T cells and tumor-infiltrating lymphocytes (TIL). PBMCs of a subject can be obtained, and CART targeting an antigen of WBC (e.g., CD19) can be prepared using various methods described in the present disclosure. In embodiments, the CAR T cells can be Coupling Component cells described in FIG. 52. The subject can then be lymphodepleted. TILs can be prepared using various methods. An example of the method is the preparation of TIL 102. For example, after excision, the metastasized tumor is digested into suspensions of single cells in 24 well plates. These suspensions are then cultured in the presence of IL-2. In embodiments, the cultures are tested for recognition of autologous melanoma cells (for example, melanoma cell lines or freshly frozen tumor digest, and if not available, a panel of HLA-matched allogeneic tumor cell lines) by measuring IFNγ secreted in the medium using an IFNγ ELISA. In embodiments, the selection step for tumor reactivity can be omitted. TIL cultures are then expanded to treatment levels by stimulation with a soluble anti-CD3 monoclonal antibody, a high concentration of IL-2, and irradiated allogeneic feeder cells. After the TIL cultures are purified to obtain the product cells, the product cells are ready to be introduced with CAR T cells that enhance TIL expansion in the subject. Information on preparations of TILs may be found in International Application NOs: WO2018/081473 and WO201S/094167 and Molecular Oncology, Volume 9, Issue 10, December 2015, Pages 1918-1935, all of which are incorporated herein by reference in their entirety.

The present disclosure describes a method of enhancing T cell response caused by a PBMs, for example, a bispecific antibody such as a BITE®, treating a subject with cancer, and/or enhancing anti-tumor activities of the PBM, the method comprising: administering an effective amount of a first PBM targeting a tumor cell, thereby causing T cell response or NK cell response; and administering an effective amount of a second PBM targeting a WBC antigen, thereby enhancing the T cell response or NK cell response, wherein the T cell response or NK cell response is greater than that of a subject administered the effective amount of the first PBM without the second PBM.

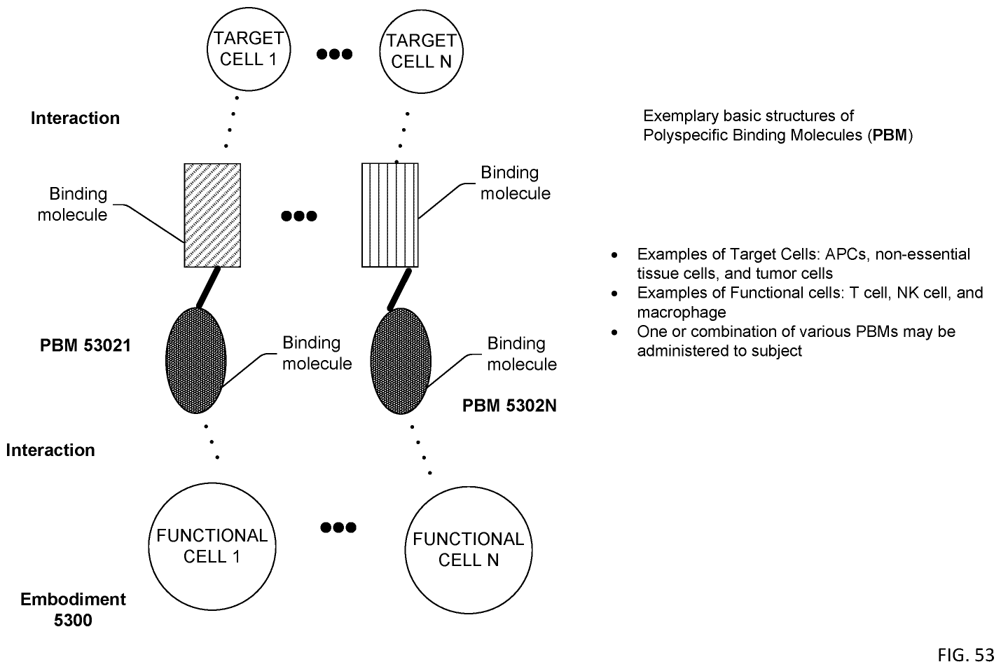

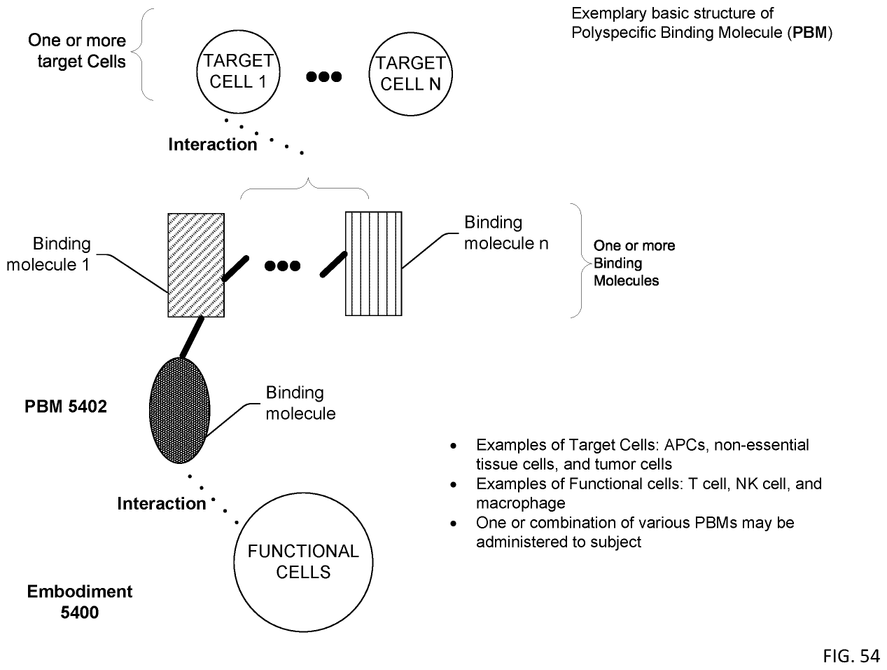

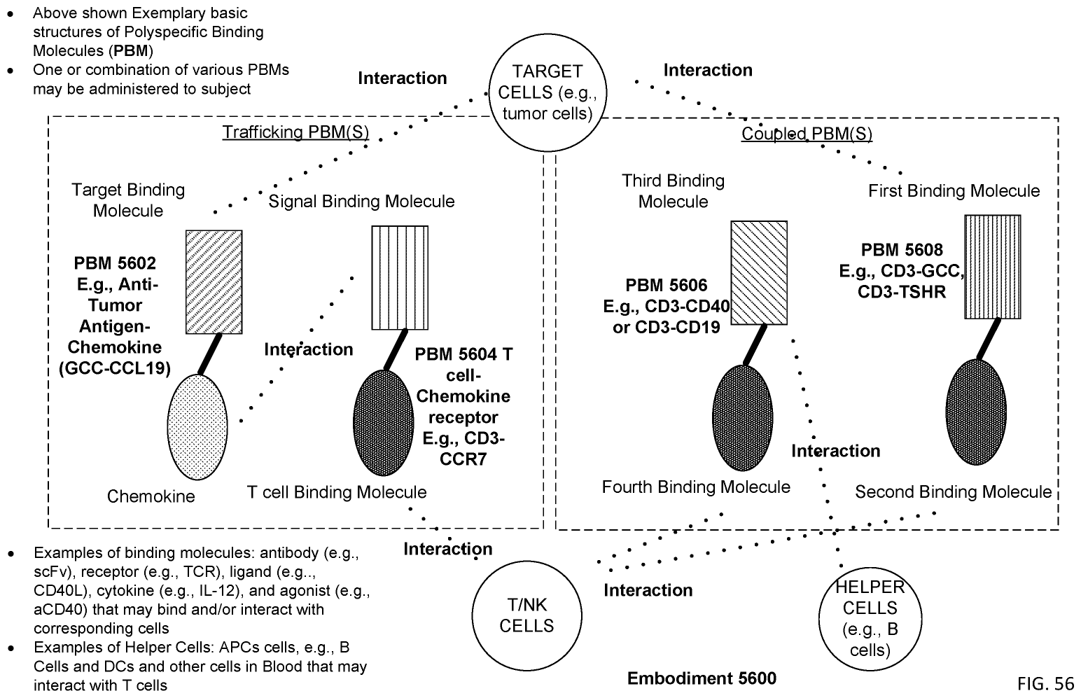

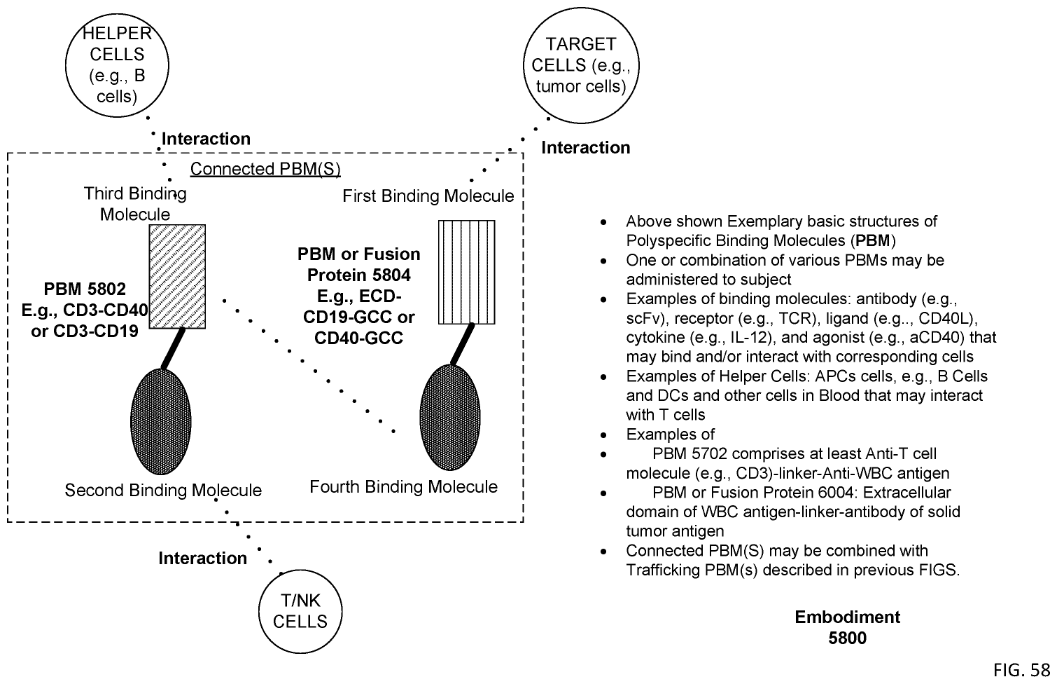

In embodiments, the first PBM comprises PBM 53021, PBM 5302N, PBM 5402, PBM 5504, PMB 5608, or PMB 5704, as shown in FIGS. 53-58. In embodiments, the second PBM comprises PBM 53021, PBM 5302N, PBM 5402, PBM 5502, PBM 5606, or PBM 5702, as shown in FIGS. 53-58. As shown in FIG. 53, there are N (number) of PBMs and N (number) of functional cells. As an example, PBM 5302N is the N^{th} PBM. In embodiments, the first PBM comprises a binding molecule and a second binding molecule. In embodiments, the second PBM comprises a third binding molecule and a fourth binding molecule. In embodiments, the binding molecules of the PBMs bind to the respective cells, for example, the helper cells (B and DC cells), the target cells such as tumor cells, and T or NK cells. The PBMs can be used together or separately. The PBMs can be used as integrated and/or coupled PBMs

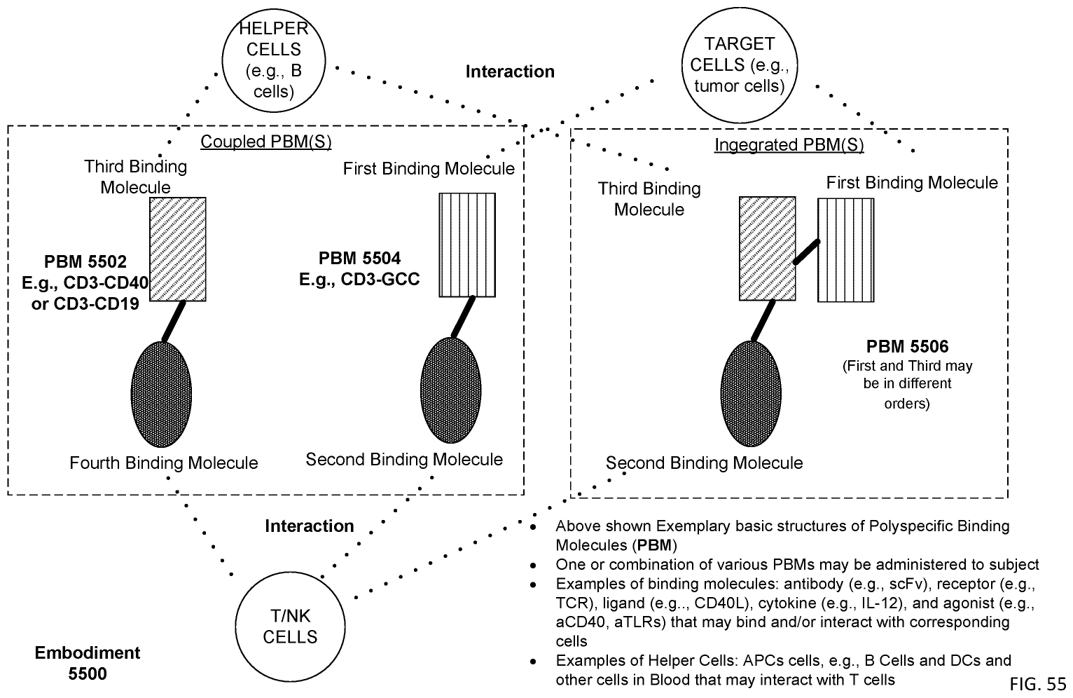

As an example, as shown in FIG. 55, the PBM of the integrated PBM can include at least three binding molecules. At least one binding molecule of the integrated PBM binds a target cell, for example a tumor cell; at least one binding molecule of the integrated PBM binds a helper cell; and at least one binding molecule of the integrated PBM binds T or NK cells. As also shown in FIG. 55, the PBMs of the coupled PBMs include two different PBMs, each comprising at least two binding molecules. At least one binding molecule of the coupled PBMs binds a target cell, for example a tumor cell; at least one binding molecule of the coupled PBMs binds a helper cell; and at least one binding molecule of the coupled PBMs, for example two binding molecules, of the couple PBMs bind T or NK cells.

In embodiments, the PBMs can also be used together as integrated and coupled PBMs.

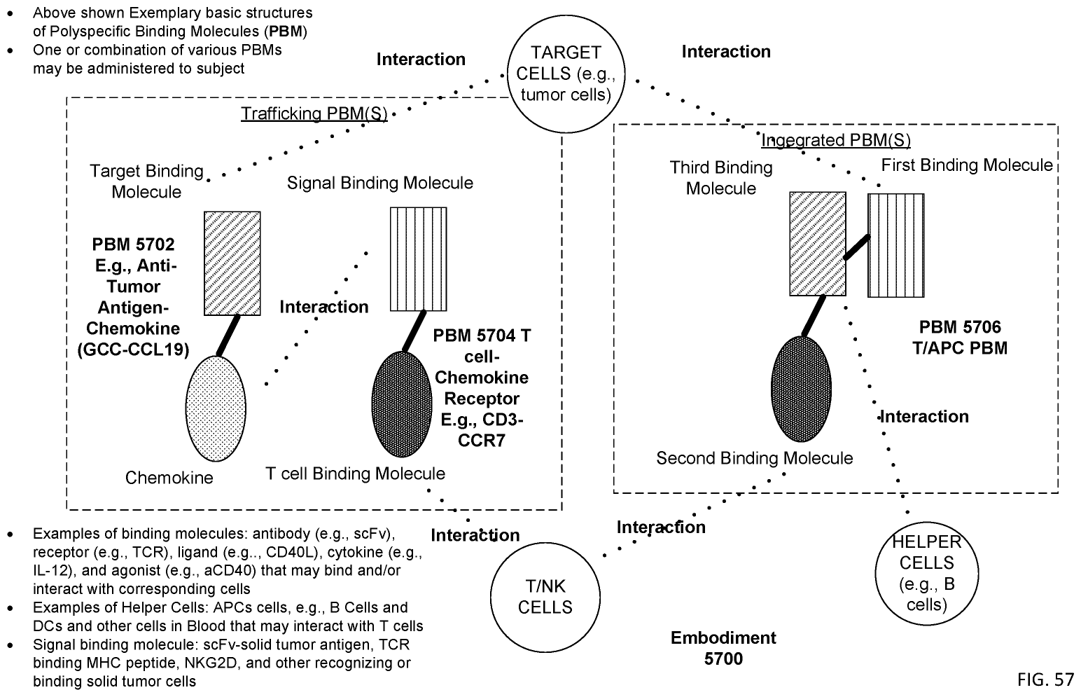

The present disclosure describes a method of enhancing T cell response caused by a PBM, such as bispecific binding molecule (e.g., BiTE®), treating a subject with cancer, and/or enhancing anti-tumor activities of the PBM, the method comprising: administering an effective amount of a PBM targeting a tumor cell, wherein the PBM comprises a first binding molecule, a second binding molecule, and a third binding molecule, wherein the T cell response or NK cell response is greater than that of a subject administered the effective amount of the PBM without the third PBM that binds or interacts with a WBC, for example, the PBM comprises PBM 5506 or 5706, as shown in FIGS. 55 and 57.

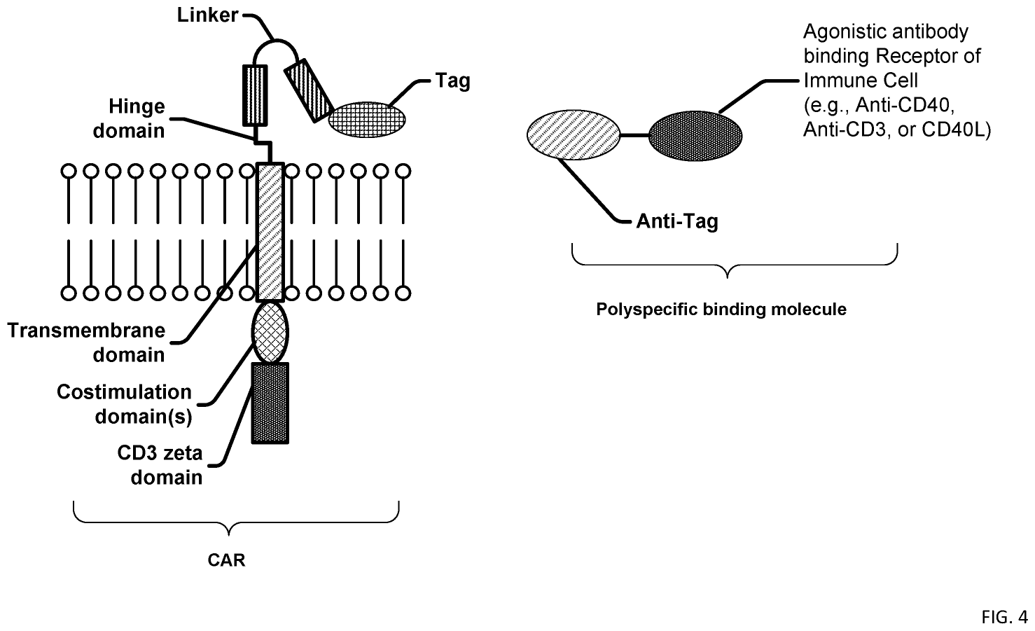

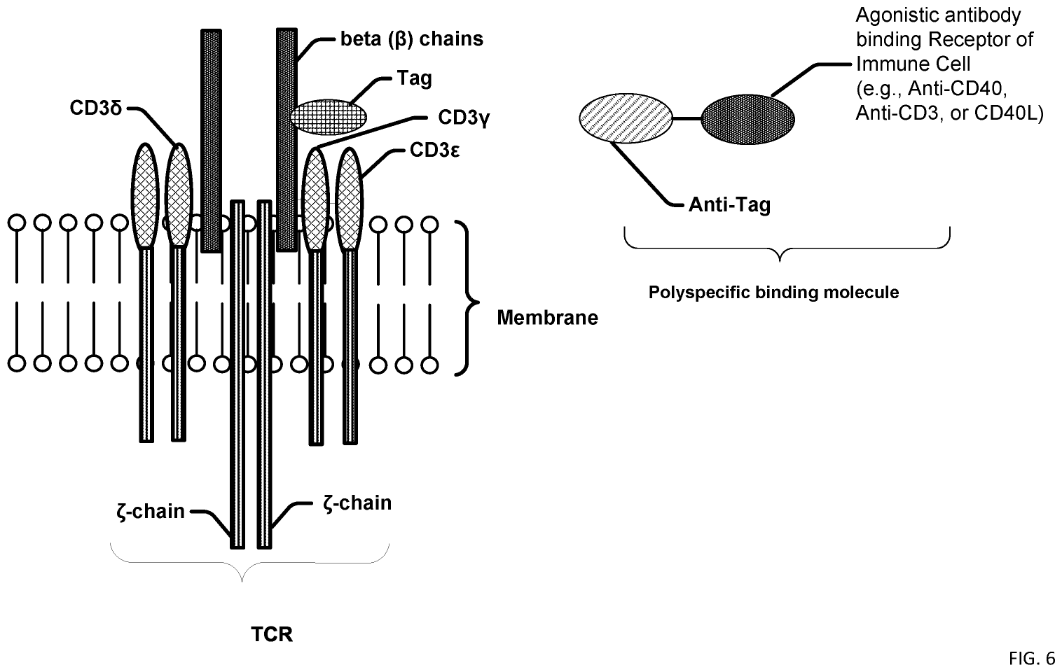

In embodiments, the PBM comprises at least two binding molecules comprising an antibody, a receptor, a ligand, a cytokine, or an agonist that binds or interact with a solid tumor, a T cell, an APC, and/or a WBC. In embodiments, the binding molecule binds a tag as shown in FIGS. 4-6 and 22. In embodiments, the binding molecule binds a WBC antigen. In embodiments, the binding molecule binds a B cell antigen. In embodiments, the binding molecule binds a solid tumor antigen. In embodiments, the binding molecule binds a T cell or an NK. In embodiments, the binding molecule binds a cytokine. In embodiments, the binding molecule binds a tag. In embodiments, the antibody comprises or is an scFv. In embodiments, the receptor comprises or is a TCR. In embodiments, the ligand comprises or is a CD40L. In embodiments, the cytokine comprises or is IL-12. In embodiments, the agonist comprises or is a CD40 agonist.

In embodiments, the first binding molecule binds a tumor antigen (e.g., a solid tumor antigen), the second binding molecule binds a T/NK cell (e.g., CD3 or CD40), the third binding molecule binds a WBC (e.g., a B cell), and the fourth binding molecule binds the T/NK cell (e.g., CD3 or CD40).

The present disclosure describes a method of enhancing T cell response caused by a PBM, for example a bispecific binding molecule (e.g., BiTE®), treating a subject with cancer, and/or enhancing anti-tumor activities of the PBM, the method comprising: administering an effective amount of a PBM comprising a target binding molecule that binds or interacts with a tumor cell and a chemokine; and administering an effective amount of a PBM comprising a signal binding molecule binding the chemokine (e.g., a chemokine receptor) and a binding molecule that binds or interacts with a T/NK cell.

The present disclosure describes a method of enhancing T cell response caused by a PBM, for example a bispecific binding molecule (e.g., BITE®), treating a subject with cancer, and/or enhancing anti-tumor activities of the PBM, the method comprising: administering an effective amount of a PBM comprising a target binding molecule that binds or interacts with a tumor cell and an extracellular domain of a WBC antigen; and administering an effective amount of a PBM comprising a binding molecule binding the WBC (e.g., CD19) and a binding molecule that binds or interacts with a T/NK cell.

In embodiments, the cells and population of cells described herein, such as the modified cells, are used in autologous CAR T cell therapy. In embodiments, the CAR T cell therapy is allogenic CAR T cell therapy, TCR T cell therapy, and NK cell therapy.

Embodiments relate to a composition comprising a population of cells including T cells comprising the CAR described herein.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples but rather should be construed to encompass any and all variations which become evident as a result of the teaching provided herein.

### EXEMPLARY EMBODIMENTS

The following are exemplary embodiments:

1. A fusion protein, such as a PBM, comprising:
a first antigen binding domain targeting a receptor of a first immune cell;
a second antigen binding domain targeting a receptor of a second immune cell; and
a third antigen binding domain targeting a tumor antigen.
2. A composition comprising:
a first fusion protein, such as a PBM, comprising a first antigen binding domain targeting a receptor of a first immune cell and a second antigen binding domain targeting a tumor antigen; and
a second fusion protein comprising a first antigen binding domain targeting a receptor of a second immune cell and a second antigen binding domain targeting a tumor antigen.
3. The composition of embodiment 2, wherein the first immune cell is a T cell, and the second immune cell is a DC.
4. The fusion protein of any preceding suitable embodiments, wherein the fusion protein is a bispecific or a trispecific antibody.
5. The fusion protein of any preceding suitable embodiments, wherein the receptor of the first immune cell and the receptor of the second immune cell are selected from receptors in Table 3 below.

TABLE 3

| Immune Cell | Receptors |
|---|---|
| monocyte | CD16, CD32, CD64, Mannose receptor (MR), |
| | Scavenger receptor (SR), Toll-like receptor |
| | (TLR), Phosphatidylserine receptor (PSR), |
| | CD14, CD40 |
| NK cell | CD16, NKp46, NKp30, NKp44, NKp80, NKG2D, |
| | KIR-S, CD94/NKG2C, CRACC, Ly9, CD84, |
| | NTBA, CD3Z, 41BB, CD28, 2B4 |
| imDC | Complement receptor, FcR, MR, TLR |
| Basic granulocyte | FcεRI |
| Acid granulocyte | FcεRI |
| Mast cells | FcεRI |
| | FcγRIII |
| Neutrophil | Dectin-1, Mac-1, TREM-1, TLR1, TLR2, TLR3, |
| | TLR4, TLR5, TLR6, TLR7, TLR8, TLR9, TLR10, |
| | NOD1, NOD2, CR4, CR1(CD35), FcγR |
| T cell | CD3, CD28, 41BB, OX40 |
| monocyte | CD16, CD32, CD64, Mannose receptor (MR), |
| | Scavenger receptor (SR), Toll-like receptor |
| | (TLR), Phosphatidylserine receptor (PSR), |
| | CD14, CD40 |
| NK cell | CD16, NKp46, NKp30, NKp44, NKp80, NKG2D, |
| | KIR-S, CD94/NKG2C, CRACC, Ly9, CD84, NTBA, |
| | CD3Z, 41BB, CD28, 2B4 |
| imDC | Complement receptor, FcR, MR, TLR |
| Basic granulocyte | FcεRI |
| Acid granulocyte | FcεRI |
| Mast cells | FcεRI, FcγRIII |
| Neutrophil | Dectin-1, Mac-1, TREM-1, TLR1, TLR2, TLR3, |
| | TLR4, TLR5, TLR6, TLR7, TLR8, TLR9, TLR10, |
| | NOD1, NOD2, CR4, CR1(CD35), FcγR |
| T cell | CD3, CD28, 41BB, OX40 |

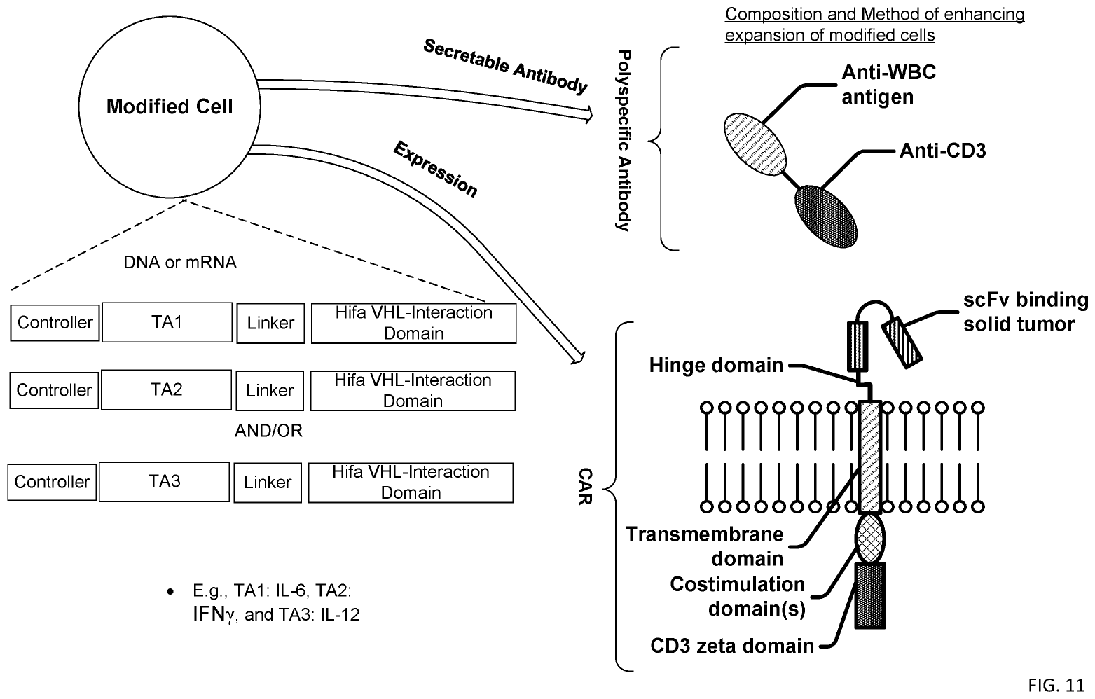

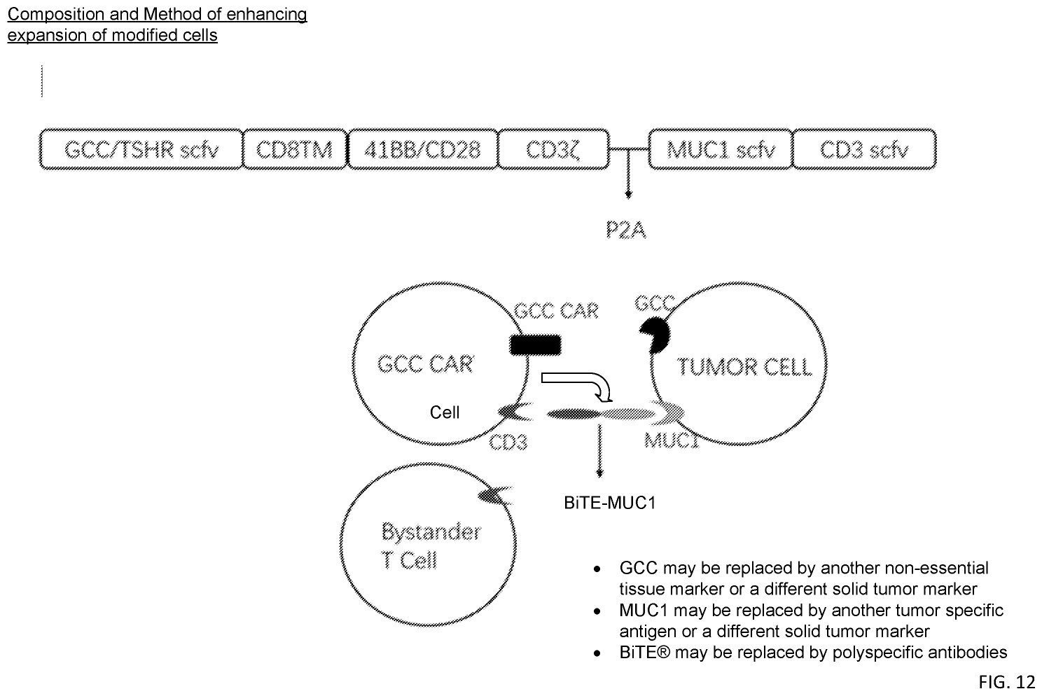

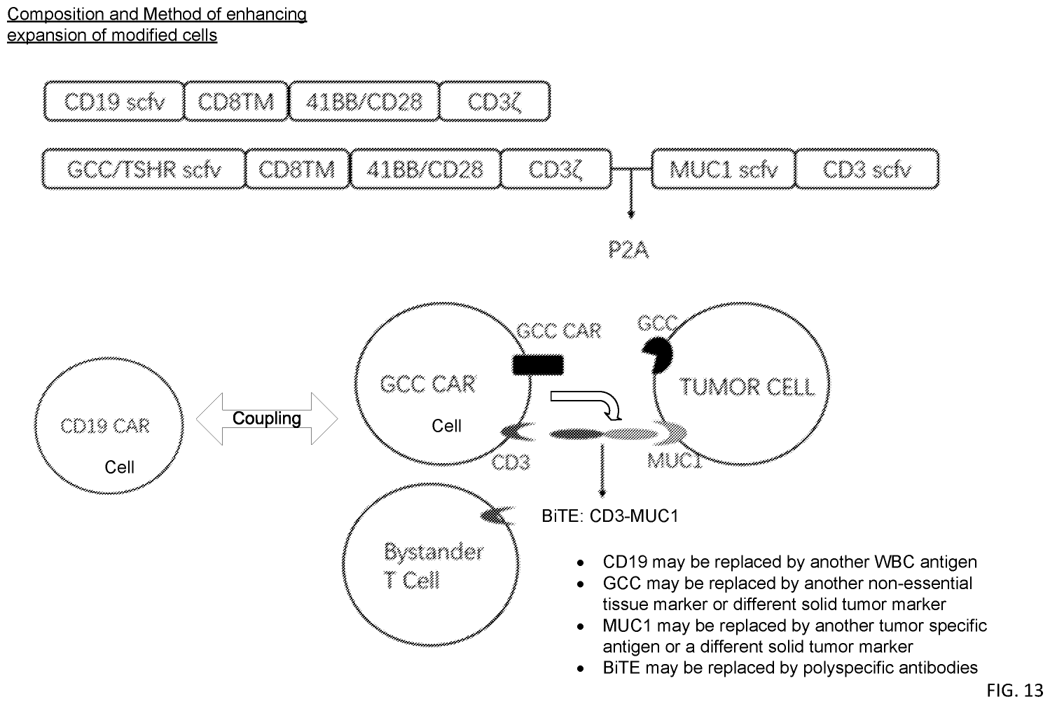

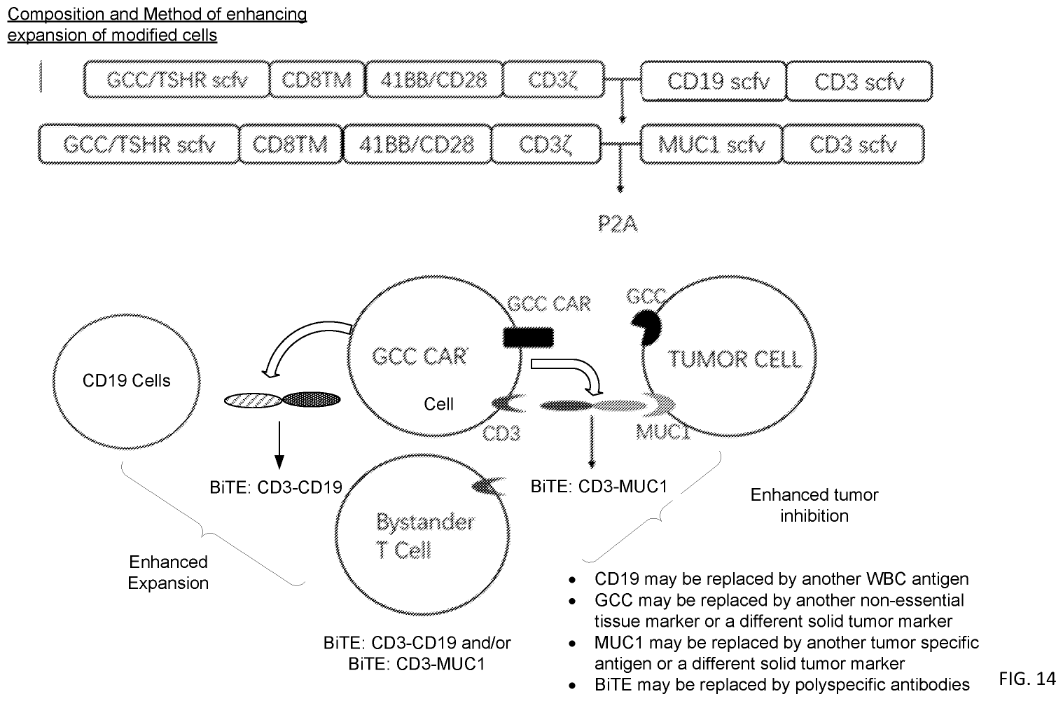

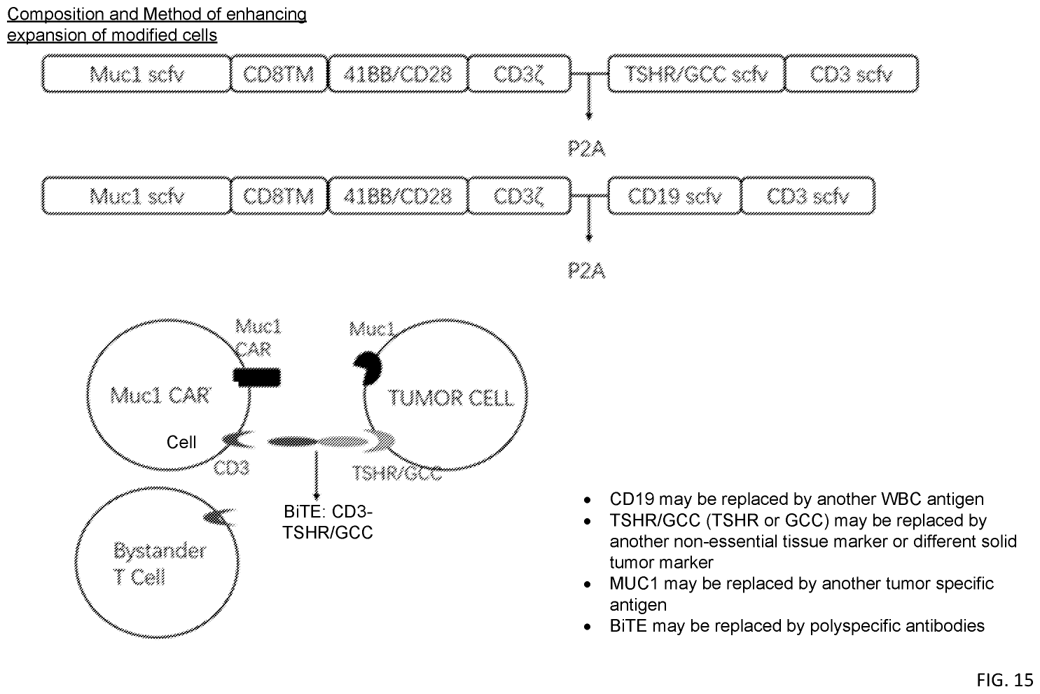

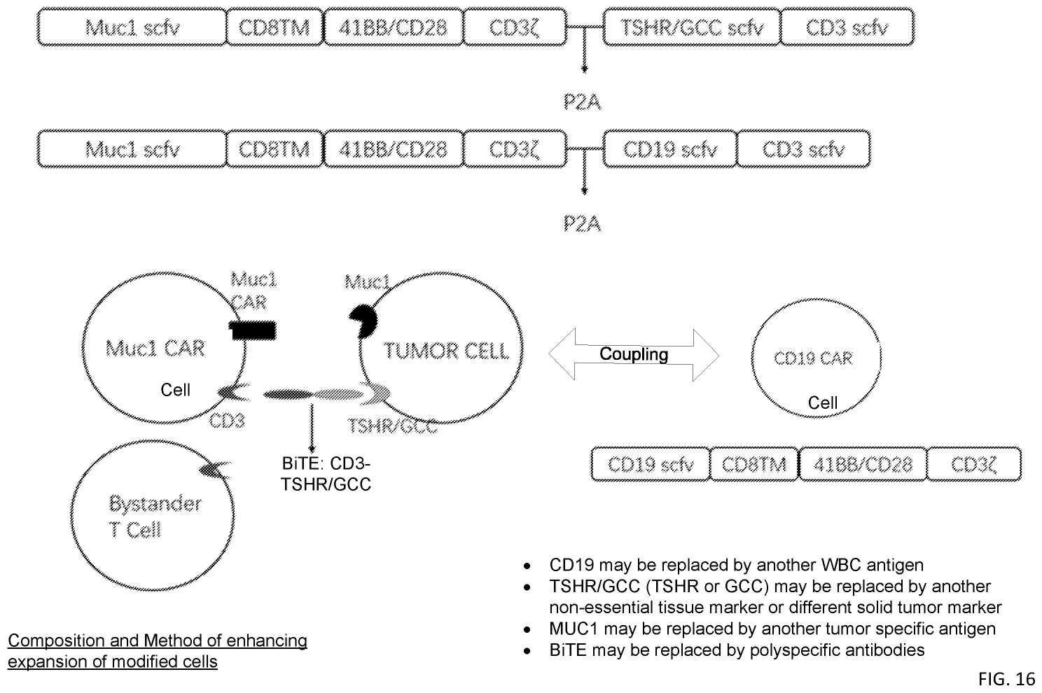

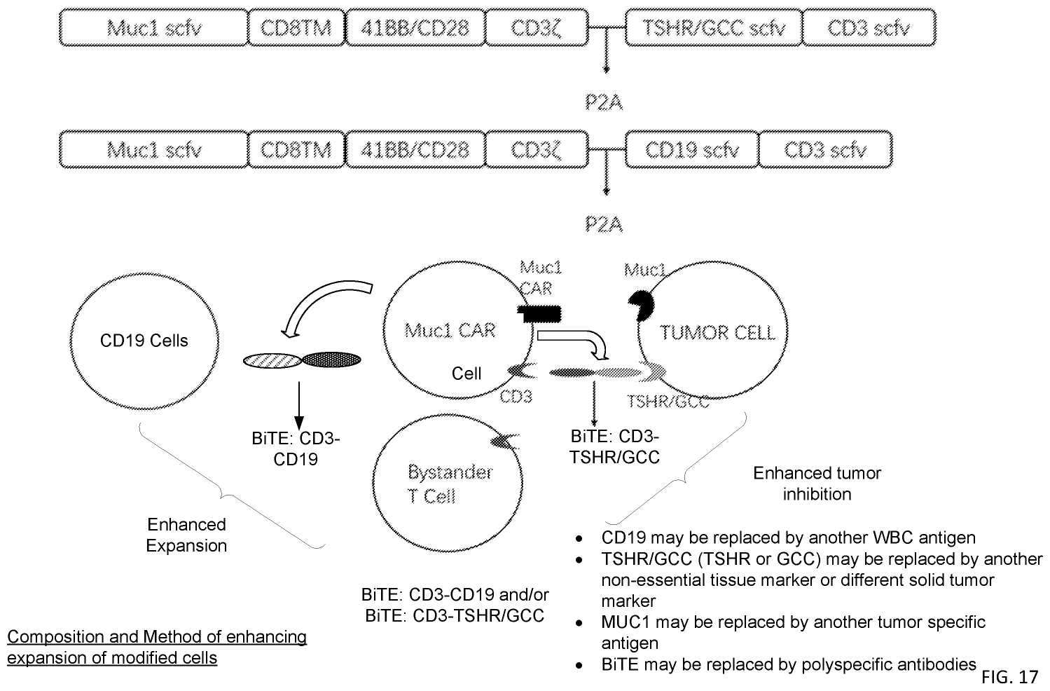

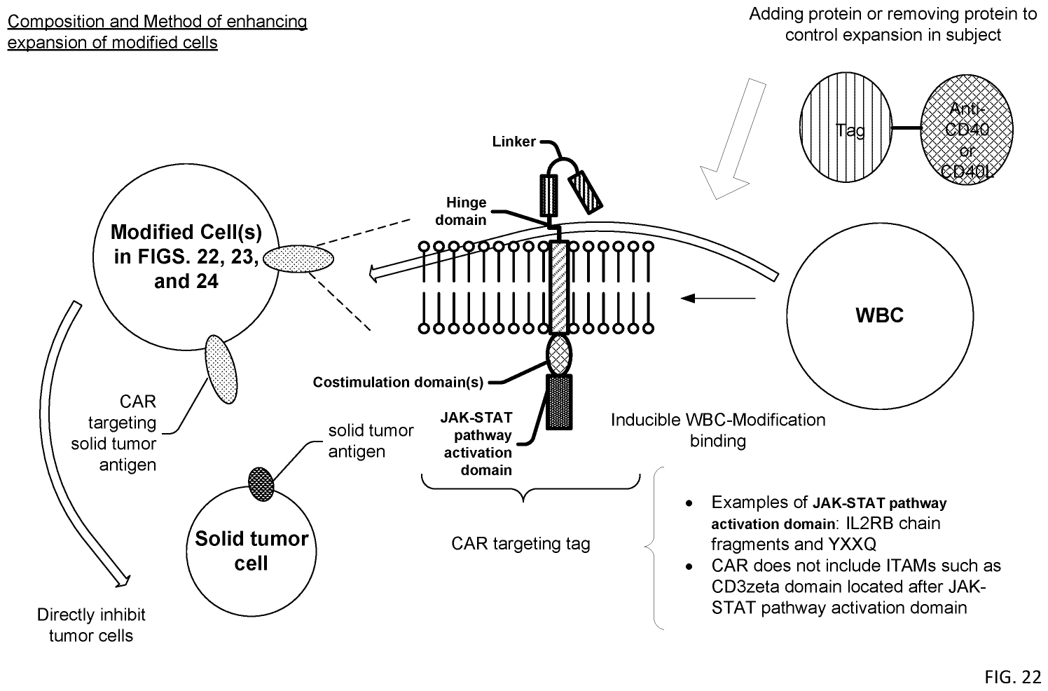

6. The fusion protein of any preceding suitable embodiments, wherein the fusion protein further comprises a therapeutic agent (such as a cytokine). Additional information regarding antibody cytokine fusion proteins can be found at J Biotechnol. 2018 Apr. 10; 271: 29-36, which is incorporated by reference in its entirety.
7. The fusion protein of embodiment 6, wherein the therapeutic agent comprises or is a cytokine or one or more anti-tumor molecules such as chemotherapy payload.
8. The fusion protein of embodiment 7, wherein the cytokine comprises or is at least one or more of IL-12, IL-6, and IFNγ.
9. The fusion protein of any preceding suitable embodiments, wherein the first antigen binding domain comprises an agonistic antibody corresponding to the receptor of the first immune cell, and/or the second antigen binding domain comprises an agonistic antibody corresponding to the receptor of the second immune cell.
10. A polynucleotide encoding the fusion protein of any preceding suitable embodiments.
11. A host cell comprising the fusion protein of any preceding suitable embodiments.
12. A modified cell comprising the fusion protein of any preceding suitable embodiments, and/or the modified cell is engineered to express one or more molecules (e.g., cytokines).
13. A method for treating or cause a T cell response in a subject having cancer, the method comprising administering an effective amount of the composition comprising the modified cell of embodiment 12.
14. A method for treating cancer in a subject, causing a T cell response in the subject or enhancing the T cell response in the subject, the method comprising administering an effective amount of the composition comprising modified cells of embodiment 12. As used throughout this application, “causing a T cell response” includes stimulating a T cell response.
15. A method for treating or causing a T cell response in a subject having cancer, the method comprising administering an effective amount of the composition comprising fusion proteins of any preceding suitable embodiments.
16. A method for treating cancer, causing a T cell response in a subject having the cancer, or enhancing the T cell response, the method comprising administering an effective amount of the composition comprising fusion proteins of any preceding suitable embodiments
17. A method for enhancing or causing a T cell response, the method comprising administering an effective amount of the composition or the fusion protein of any preceding suitable embodiments and a modified cell comprising a CAR or TCR, wherein the fusion protein is bispecific or trispecific antibody that binds CD3 and a tag, and wherein the CAR or TCR is associated with the tag.
18. A method for enhancing or causing a T cell response, the method comprising administering an effective amount of the composition of a fusion protein of any preceding suitable embodiments and a modified cell comprising a CAR or TCR, wherein the fusion protein comprises a binding domain binding bind CD3 and comprises a tag, and wherein the CAR or TCR binds the tag.
19. A method for enhancing or causing a T cell response, the method comprising administering an effective amount of a composition or a modified cell that comprises a polynucleotide encoding a PBM (e.g., polyspecific antibody) of any preceding suitable embodiments and a polynucleotide encoding a CAR/TCR that binds a solid tumor antigen. (See Example in FIG. 11)
20. The method of embodiment 19, wherein the PBM (e.g., polyspecific antibody) comprises a bispecific binding molecule, such as a bispecific antibody, for example BITE®, comprising a binding domain binding a WBC (white blood antigen) antigen and a binding domain binding CD3.
21. The method of embodiment 19, wherein the modified cell further comprises a polynucleotide encoding one or more therapeutic agents (e.g., IL-12, IL-6, and IFNγ).
22. A method for enhancing or causing a T cell response, the method comprising administering an effective amount of a composition of a modified cell comprising a polynucleotide encoding a PBM (e.g., polyspecific antibody) of any preceding suitable embodiments and a polynucleotide encoding a CAR/TCR that binds a non-essential tissue antigen (e.g., GCC, TSHR, PAP), the PBM (e.g., polyspecific antibody) binding CD3 and a tumor specific antigen (e.g., MUC1 and EGFRVIII), the modified cell expressing and secreting the PBM (e.g., polyspecific antibody). (See Example in FIG. 12)
23. The method of embodiment 22, wherein the composition further comprises modified cells comprising a polynucleotide encoding a CAR (e.g., CD19 CAR). (See Example in FIG. 13) A WBC CAR comprises a CAR that binds a WBC antigen; or
the method of embodiment 22, wherein the modified cell further expresses and secretes a PBM (e.g., polyspecific antibody) that binds CD19 and CD3. (See Example in FIG. 14)
24. A method for enhancing or causing a T cell response, the method comprising administering an effective amount of a composition of a modified cell that comprises a polynucleotide encoding a PBM (e.g., polyspecific antibody) of any preceding suitable embodiments and a polynucleotide encoding a CAR/TCR that binds a tumor specific antigen (e.g., MUC1 and EGFRVIII), the PBM (e.g., polyspecific antibody) binding CD3 and a non-essential tissue antigen (e.g., GCC, TSHR, PAP), the modified cell expressing and secreting the PBM (e.g., polyspecific antibody). (See Example in FIG. 15)
25. The method of embodiment 24, wherein the composition further comprises modified cells comprising a polynucleotide encoding a WBC CAR (e.g., CD19 CAR). (See Example in FIG. 16)
26. The method of embodiment 25, wherein the modified cell further expresses and secretes a PBM (e.g., polyspecific antibody) that binds CD19 and CD3 (See Example in FIG. 17).
27. A method for enhancing or causing a T cell response, the method comprising administering an effective amount of a composition of a modified cell that comprises a CAR or TCR binding a solid tumor antigen and a composition of a modified cell engineered to express and secrete the solid tumor antigen.
28. The method of embodiment 27, wherein the solid tumor antigen is expressed by tumor cells.
29. The method of embodiment 27, wherein the solid tumor antigen is a non-essential tissue antigen.
30. The modified cell of any of the preceding embodiments, wherein the enhanced expression and/or function of the one or more molecules is implemented by introducing a nucleic acid encoding the one or more molecules and/or the binding molecule (e.g., CAR and TCR), which is present in the modified cell in a recombinant DNA construct, in an mRNA, or in a viral vector.
31. The modified cell of embodiment 30, wherein the nucleic acid is an mRNA, which is not integrated into the genome of the modified cell.
32. The modified cell of embodiment 30, wherein the nucleic acid is associated with an oxygen-sensitive polypeptide domain.
33. The modified cell of embodiment 32, wherein the oxygen-sensitive polypeptide domain comprises HIF VHL binding domain.
34. The modified cell of embodiment 30, wherein the nucleic acid is regulated by a promoter comprising a binding site for a transcription modulator that modulates the expression and/or secretion of the therapeutic agent in the cell.
35. The modified cell of embodiment 34, wherein the transcription modulator is or includes Hif1a, NFAT, FOXP3, and/or NFkB.
36. The modified cell of any of the preceding suitable embodiments, wherein the one or more molecules comprise at least one of G-CSF or GM-CSF, or a combination thereof.
37. The modified cell of any of the preceding suitable embodiments, wherein the one or more molecules comprise at least one of a receptor of G-CSF or GM-CSF, or a combination thereof.
38. The modified cell of any of the preceding suitable embodiments, wherein the one or more molecules comprise at least one of IL-33, IL-1β, TNFα, MALP-2, IL1, or IL17.
39. A polynucleotide encoding the one or more molecules of any preceding suitable embodiments and an antigen binding molecule.
40. The modified cell of any of the preceding suitable embodiments, wherein the modified cell comprises an antigen binding molecule, the antigen binding molecule comprises or is a chimeric antigen receptor (CAR) comprising an antigen-binding domain, a transmembrane domain, and an intracellular signaling domain.
41. The modified cell of embodiment 40, wherein the antigen-binding domain binds a tumor antigen selected from a group consisting of: TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGSS, HMWMAA, o-acetyl-GD2, Folate receptor beta, TEM1/CD248, TEM7R, CLDN6, GPRC5D, CXORF61, CD97, CD179a, ALK, Polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, and IGLL1.
42. The modified cell of embodiment 40 or 41, wherein the intracellular signaling domain comprises a co-stimulatory signaling domain, or a primary signaling domain and a co-stimulatory signaling domain, wherein the co-stimulatory signaling domain comprises a functional signaling domain of a protein selected from the group consisting of CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, NKG2D, or a combination thereof.
43. The modified cell of any one of suitable preceding embodiments, wherein the modified cell comprises an antigen binding molecule, the antigen binding molecule comprising or is a modified TCR.
44. The modified cell of embodiment 43, wherein the TCR is derived from spontaneously occurring tumor-specific T cells in patients.
45. The modified cell of embodiment 44, wherein the TCR binds a tumor antigen.
46. The modified cell of embodiment 45, wherein the tumor antigen comprises CEA, gp100, MART-1, p53, MAGE-A3, or NY-ESO-1.
47. The modified cell of embodiment 45, wherein the TCR comprises TCRγ and TCRδ chains or TCRα and TCRβ chains, or a combination thereof.
48. The modified cell of any of the preceding suitable embodiments, wherein the cell is an immune cell (e.g., an immune effector cell), or the immune cell is a T cell or an NK cell.
49. The modified cell of embodiment 48, wherein the immune effector cell is a T cell.
50. modified cell of embodiment 49, wherein the T cell is a CD4+ T cell, a CD8+ T cell, or a combination thereof.
51. The modified cell of any of the preceding suitable embodiments, wherein the cell is a human cell.
52. The modified cell of any of the preceding suitable embodiments, wherein the modified cell comprises a nucleic acid encoding a binding molecule and a dominant negative form of an inhibitory immune checkpoint molecule or a receptor thereof.
53. The modified cell of embodiment 52, wherein the inhibitory immune checkpoint molecule is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRD, natural killer cell receptor 2B4 (2B4), and CD 160.
54. The modified cell embodiment 52, wherein inhibitory immune checkpoint molecule is modified PD-1.
55. The modified cell of embodiment 54, wherein the modified PD-1 lacks a functional PD-1 intracellular domain for PD-1 signal transduction, interferes with a pathway between PD-1 of a human T cell and PD-L1 of a certain cell, comprises or is a PD-1 extracellular domain or a PD-1 transmembrane domain, a modified PD-1 intracellular domain comprising a substitution or deletion as compared to a wild-type PD-1 intracellular domain, or comprises or is a soluble receptor comprising a PD-1 extracellular domain that binds to PD-L1 of a certain cell, or a combination thereof. In embodiments, interfering with the pathway between two molecules include interfering with the interaction between two molecules, such as PD-1 and PD-L1.
56. The modified cell of any preceding suitable embodiments, wherein the modified cell is engineered to express and secrete one or more therapeutic agents such as a cytokine, for example, IL-6 and/or IFNγ.
57. The modified cell of embodiment 56, wherein the therapeutic agent comprises IL-15 or IL-12, a combination thereof, a small protein or the therapeutic agent comprises a recombinant or native cytokine, and/or the small protein comprises IL-12, IL-6 and/or IFNγ.
58. The modified cell of any preceding suitable embodiments, wherein the modified cell is derived form a healthy donor or the subject having the cancer.
59. The modified cell of embodiment 58, wherein the modified ell has a reduced expression of endogenous T Cell Receptor Alpha Constant (TRAC) gene.
60. The modified cell of any preceding suitable embodiments, wherein the modified cell comprises a first CAR binding a white blood antigen and a second CAR binding a solid tumor antigen.
61. The modified cell of any preceding suitable embodiments, wherein the modified cell comprises a bispecific CAR binding a WBC antigen and a solid tumor antigen.
62. A pharmaceutical composition comprising a population of the modified cells of any one of embodiments 1-61 and a population of additional modified cells, wherein the modified cells of any of embodiments 1-61 bind a first antigen, and the additional modified cells bind a second antigen, which is different form the first antigen.
63. The pharmaceutical composition of embodiment 62, wherein the first antigen is a white blood cell antigen, and the second antigen is a solid tumor antigen.
64. The pharmaceutical composition of embodiment 62, wherein the second antigen is a white blood cell antigen, and the first antigen is a solid tumor antigen.
65. The pharmaceutical composition of embodiments 63 or 64, wherein the white blood cell antigen is CD19, CD22, CD20, BCMA, CD5, CD7, CD2, CD16, CD56, CD30, CD14, CD68, CD11b, CD18, CD169, CD1c, CD33, CD38, CD138, or CD13.
66. The pharmaceutical composition of embodiments 63 or 64, wherein the WBC is CD19, CD20, CD22, or BCMA.
67. The pharmaceutical composition of embodiments 63 or 64, wherein the solid tumor antigen is tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, CLDN18.2, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, B7-H3, or EGFR.
68. The pharmaceutical composition of embodiments 63 or 64, wherein the solid tumor antigen comprises tumor associated MUC1, ACPP, TSHR, GUCY2C, UPK2, CLDN18.2, PSMA, DPEP3, CXCR5, B7-H3, MUC16, SIGLEC-15, CLDN6, Muc17, PRLR, or FZD10.
69. A method of eliciting or enhancing T cell response, treating a subject in need thereof or enhancing cancer treatment thereof, the method comprising administering an effective amount of the pharmaceutical composition of any of embodiments 62-68.
70. A method for treating a subject having cancer, wherein the method comprises: inducing TILs out of tumor tissues using a mobilizer like GCSF or GMCSF; and administering an effective amount of CAR T cells targeting a WBC antigen (e.g., CD19) or a bispecific antibody (e.g., Anti-CD19/Anti-D3) targeting the WBC antigen or a T cell antigen such that the TILs are expanded in the human body, in a non-tumor environment, to achieve anti-cancer effect.
71. The method of embodiment 70, wherein the CAR T cells or the bispecific antibody comprises a cytokine.
72. A modified cell comprising: a chimeric antigen receptor (CAR), wherein the CAR comprises an extracellular domain, a transmembrane domain, and an intracellular domain, the extracellular domain comprising an extracellular domain of a receptor of a homing molecule, and the intracellular domain comprising an intracellular domain of a T cell activation molecule; and an additional CAR targeting a solid tumor antigen.
73. An isolated nucleic acid encoding a chimeric antigen receptor (CAR), wherein the CAR comprises an extracellular domain, a transmembrane domain, and an intracellular domain, the extracellular domain comprising an extracellular domain of a receptor of a homing molecule, and the intracellular domain comprising an intracellular domain of a T cell activation molecule.
74. A population of CAR cells comprising the nucleic acid embodiment 73 or the modified cell of embodiment 72.
75. A pharmaceutical composition comprising the population of the CAR cells of embodiment 74.
76. A method of causing a T cell response in a subject in need thereof and/or treating a tumor of the subject, the method comprising administering an effective amount of the composition of embodiment 75 to the subject.
77. A modified cell comprising a chimeric antigen receptor (CAR), wherein the CAR comprises an extracellular domain, a transmembrane domain, and an intracellular domain, the extracellular domain comprising an extracellular domain of a receptor of a homing molecule, and the intracellular domain comprising an intracellular domain of a T cell activation molecule.
78. The isolated nucleic acid, the population of CAR cells, the pharmaceutical composition, or the modified cell of any of embodiments 72-77, wherein the homing molecule is CCR2, CCR4, CXCR3, CCR6, ICAM3, CCR7, LFA-3, CCR1, CCR3, or CCR5.
79. The isolated nucleic acid, the population of CAR cells, the pharmaceutical composition, or the modified cell of any of embodiments 72-77, wherein the T cell activation molecule is CD27, CD28, 4-IBB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, or B7-H3.
80. The isolated nucleic acid, the population of CAR cells, the pharmaceutical composition, or the modified cell of any of embodiments 72-77, wherein the transmembrane membrane is CD28, CD3 epsilon, CD45, CD4, CD5, CD8, CD9, CD16, CD22, CD33, CD37, CD64, CD80, CD86, CD134, CD137, or CD154.
81. The isolated nucleic acid, the population of CAR cells, the pharmaceutical composition, or the modified cell of any of embodiments 74-80, wherein the modified cell or cells comprise an additional CAR binds to an antigen.
82. The isolated nucleic acid, the population of CAR cells, the pharmaceutical composition, or the modified cell of embodiment 81, wherein the intracellular domain of the additional CAR comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof.
83. The isolated nucleic acid, the population of CAR cells, the pharmaceutical composition, or the modified cell of embodiment 81, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-a (FR-a), CD19, FZD10, TSHR, PRLR, Muc 17, GUCY2C, CD207, CD3, CD5, B-Cell Maturation Antigen (BCMA), or CD4.
84. The isolated nucleic acid, the population of CAR cells, the pharmaceutical composition, or the modified cell of any of embodiments 72-83, wherein the transmembrane domain of the CAR comprises a transmembrane domain of a T cell receptor.
85. A method of enhancing T cell response in body of a subject, the method comprising: administering to the subject an effective amount of a population of lymphocytes comprising an antigen binding molecule and one or more agents that enhance expansion of lymphocytes comprising the antigen binding molecule in the subject.
86. The method of embodiment 85, wherein the T cell response comprises expansion or activation of the lymphocytes and/or anti-tumor efficacy of immunotherapy in the subject.
87. The method of embodiment 85, wherein the one or more agents or the lymphocytes comprise a CAR comprising an extracellular domain binding a WBC antigen, a transmembrane domain, and an intracellular domain comprising a co-stimulatory domain and a domain associated with the signaling of IL-2R (e.g., JAK-STAT domain).
88. The method of embodiment 85, wherein the one or more agents or the lymphocytes comprise a CAR comprising an extracellular domain binding a tag, a transmembrane domain, and an intracellular domain comprising a co-stimulatory domain and a JAK-STAT signaling domain or a domain associated thereof (upstream and downstream). Additional information about the JAK-STAT signaling domain can be found at Nat Med. 2018 March; 24(3): 352-359. doi:10.1038/nm.4478, which is incorporated herein by reference in its entirety.
89. The method of embodiment 88, further comprising:
administering effective amount of a fusion protein comprising a tag and anti-CD40 or CD40L.
90. The method of embodiment 87 or 88, wherein the CAR does not comprise CD3 zeta domain.
91. A polynucleotide encoding the CAR used in the method of any of embodiments 87-89.
92. A vector comprising the polynucleotide of embodiment 91.
93. A cell comprising the polynucleotide or the vector of embodiment 91 or 92.
94. A CAR of any of embodiments 87-89.
95. The method of any preceding suitable embodiments, wherein the one or more agents enhance function of a co-stimulatory signaling pathway of the lymphocytes and/or a cytokine receptor.
96. The method of embodiment 95, wherein the enhancement is inducible.
97. The method of embodiment 95, wherein the enhancement is controlled by the binding of the lymphocytes and a WBC or the binding of the CAR.
98. The method of any of embodiments 95-97, wherein the co-stimulatory signaling pathway comprises CD80 and/or CD86, CD40, or 4-1BB, and the cytokine receptor is an IL-2 receptor.
99. The method of any preceding suitable embodiments, wherein the one or more agents up-regulates or enhance maintenance of function and/or expression of CD80 and/or CD86 of the lymphocytes, and/or the one or more agents up-regulates or enhance maintenance of function and/or expression of IL-2 receptor (e.g., CD25) of the lymphocytes.
100. The method of any preceding suitable embodiments, wherein the one or more agents comprise a secretable IL-2.
101. The method of embodiment 99 or 100, wherein the up-regulation or enhancement is inducible.
102. The method of embodiment 101, wherein the up-regulation or enhancement is controlled by the binding of the lymphocytes and a WBC or binding of the CAR.
103. The method or any preceding suitable embodiments, wherein the one or more agents cause an addition or disruption of one or more genes of the lymphocytes, which is implemented by ZFN, Cas9, or TLAEN, and/or the lymphocytes further comprise a polynucleotide encoding a therapeutic agent (e.g., a cytokine) under control of a regulatory element (e.g., NFAT, HIF etc.).
104. The method of any preceding suitable embodiments, wherein the binding molecule is the CAR, the method further comprises administering to the subject an effective amount of another population of lymphocytes comprising a CAR or TCR binding a solid tumor antigen, and the number of the another population of lymphocytes is greater than the population of lymphocytes.
105. The method of any preceding embodiments, wherein the lymphocytes are T cells, DCs, macrophages, and/or NK cells.
106. The method of any preceding suitable embodiments, wherein the antigen binding molecule is CAR or TCR targeting an antigen associated with the cancer described in any preceding suitable embodiments.
107. The method of any preceding suitable embodiments, wherein the lymphocytes are T cells, and the antigen binding molecule is a CAR targeting a solid tumor antigen.
108. A method of enhancing or eliciting T cell response or enhancing treatment of or treating a subject having cancer, the method comprising: administering to the subject an effective amount of a population of lymphocytes (e.g., T cells or NKs) comprising an antigen binding molecule binding a solid tumor antigen; and activating or enhancing functions or activities of the lymphocytes' co-stimulatory signaling domain; and activating or enhancing functions or activities of IL-2 signaling pathway.
109. The method of embodiment 108, wherein activating or enhancing functions or activities of the lymphocytes' co-stimulatory signaling domain comprises activating or enhancing functions or activities of the lymphocytes' co-stimulatory signaling domain using a bispecific antibody targeting the molecule of the co-stimulatory signaling domain and a WBC antigen or a solid tumor antigen.
110. The method of embodiment 108, wherein activating or enhancing functions or activities of the lymphocytes' co-stimulatory signaling domain comprises activating or enhancing functions or activities of the lymphocytes' co-stimulatory signaling domain using a CAR binding a WBC antigen.
111. The method of embodiment 108, wherein activating or enhancing functions or activities of the lymphocytes' co-stimulatory signaling domain comprises activating or enhancing functions or activities of the lymphocytes' co-stimulatory signaling domain using a bispecific antibody targeting a solid tumor antigen/a WBC antigen and CD3 or comprising CD40L (or anti-CD40).
112. The method of any suitable preceding embodiments, further comprising: administering an effective amount of GCSF or GMCSF to the subject before administering to the subject the effective amount of the population of lymphocytes.
113. The method of any suitable preceding embodiments, further comprising: administering an effective amount of GCSF or GMCSF to the subject after administering to the subject the effective amount of the population of lymphocytes.
114. The method of any suitable preceding embodiments, further comprising: administering an effective amount of GCSF or GMCSF to the subject at the same time as administering to the subject the effective amount of the population of lymphocytes.
115. An isolated nucleic acid encoding a CAR, wherein the CAR comprises an extracellular domain, a transmembrane domain, and an intracellular domain, the extracellular domain binds an antigen, the intracellular signaling domain comprising an exogenous Signal Transducer and Activator of Transcription (STAT) 3 association motif, wherein the intracellular segment comprises an endogenous or exogenous JAK-binding motif and a STATS association motif, and wherein the intracellular signaling domain does not comprise CD3zeta domain.
116. The isolated nucleic acid of embodiment 115, wherein the exogenous JAK-binding motif can be inserted into an intracellular signaling domain, for example a cytoplasmic domain of an interleukin receptor chain.
117. The isolated nucleic acid of embodiment 115, wherein the JAK-binding motif comprises a BOX-1 motif which allows for tyrosine kinase JAK association, for example JAK1, and the JAK-binding motif can be for example amino acid 278 to 286 of NCBI RefSeq:NP_000869.1 (amino acids 13 to 21 SEQ ID NO: 5).
118. The isolated nucleic acid of embodiment 115, wherein the domain comprises one region in a polypeptide, for example which is folded into a particular structure independently of other regions and/or has a particular function. The domain can for example be the cytoplasmic portion of a molecule or a part thereof.
119. The isolated nucleic acid of embodiment 115, wherein the intracellular segment comprises an endogenous or exogenous JAK-binding motif and a STATS association motif, wherein the intracellular segment comprises more than one cytoplasmic domain, and wherein the JAK binding motif and the STATS association motif may be in the same cytoplasmic domain or may be in separate cytoplasmic domains.
120. The isolated nucleic acid of any of embodiments 115-119, wherein the exogenous STAT3 association motif is YXXQ (SEQ ID NO: 2).
121. The isolated nucleic acid of any of embodiments 115-119, wherein the exogenous STAT3 association motif is YRHQ (SEQ ID NO: 3).
122. The isolated nucleic acid sequence of any of embodiments 115 to 121, wherein the exogenous STAT3 association motif is introduced less than 100 amino acid residues from the C terminus of the CAR.
123. The isolated nucleic acid any one of embodiments 115 to 122, wherein the one or more intracellular signaling domains is or comprises the cytoplasmic domain of an interleukin receptor chain.
124. The isolated nucleic acid any one of embodiments 115 to 123, wherein the cytoplasmic domain of an interleukin receptor chain is a truncated fragment of the cytoplasmic domain comprising a JAK-binding motif and a STATS association motif.
125. The isolated nucleic acid any one of embodiments 115 to 124, wherein the STATS association motif is YXXL (SEQ ID NO: 4).
126. The isolated nucleic acid of any one of embodiments 115-125, wherein the one or more intracellular signaling domains is or comprises a cytoplasmic co-stimulatory domain.
127. The isolated nucleic acid of any one of embodiments 115 to 126, wherein the cytoplasmic co-stimulatory domain is a cytoplasmic domain of CD28, CD2, CD4, CD5, CD8a, CD8p, CD134, or CD137.
128. The isolated nucleic acid of embodiment 127, wherein the cytoplasmic domain of an interleukin receptor chain is a truncated fragment of said cytoplasmic domain comprising a tyrosine kinase association motif and a STAT association motif.
129. The isolated nucleic acid of any one of embodiments 115 to 128, wherein the interleukin receptor chain is selected from the group consisting of interleukin 2 receptor (IL-2R) beta chain and interleukin 21 receptor (IL-21 R) a chain.
130. The isolated nucleic acid of any one of embodiments 115 to 129 wherein the extracellular domain is an antigen binding region of an antibody capable of binding to the predetermined antigen.
131. The isolated nucleic acid of embodiment 130, wherein the antigen binding region of the antibody is a single chain variable fragment of said antibody.
132. The isolated nucleic acid of any one of embodiments 115 to 131, wherein the transmembrane domain is selected from the group consisting of CD28 transmembrane domain and CD8 transmembrane domain.
133. The isolated nucleic acid of any one of embodiments 115 to 131, wherein the nucleic acid encodes the CAR and further encodes a signal peptide at the N terminus.
134. A CAR encoded by the nucleic acid of any one of embodiments 115 to 133.
135. A vector comprising the nucleic acid of any of embodiments 115-133.
136. A cell that expresses the CAR of any one of suitable preceding embodiments and/or is transfected or transduced with the nucleic acid of any of suitable preceding embodiments or the vector of any of suitable preceding embodiments.
137. A composition comprising the CAR, nucleic acid, vector, or cell of any one of embodiments 115 to 136, and optionally a pharmaceutically acceptable excipient or carrier.
138. A method of making the cell of embodiment 136, transducing a cell with the nucleic acid of any of suitable preceding embodiments, or the vector of any suitable preceding embodiments.
139. A method of making the cell of embodiment 136, comprising: isolating immune cells from a mammal, optionally wherein the immune cells are T cells; transfecting or transducing the isolated immune cells, optionally T cells, with a nucleic acid encoding a CAR of any one of suitable preceding embodiments; and optionally isolating and/or expanding the CAR-expressing cells, optionally CAR-expressing T cells following transfection or transduction.
140. Use of the CAR, nucleic acid, vector, cell, or composition of any suitable preceding embodiments, for treating or preventing a disease, for expanding certain cells in a subject (e.g., T cells), and/or for decreasing in a subject the number of cells expressing a predetermined antigen.
141. A method of treating a disease in a subject, the method comprising administering to the subject in need thereof an effective amount of cells or the composition ofany suitable preceding embodiments.
142. The use or method of embodiment 140 or 141 for providing an anti-tumor immunity in a mammal.
143. A method of decreasing in a subject the number of cells expressing a predetermined antigen, the method comprising administering to the subject in need thereof an effective amount of cells according to any of suitable preceding embodiments.
144. A method of enhancing T cell response, enhancing cellular therapy, and/or enhancing anti-tumor activities in a subject having cancer, the method comprising: administering an effective amount of a population of cells of any suitable preceding embodiments; and allowing the T cells to expand and/or release a cytokine in the body of the subject, wherein: the T cell response comprises T cell expansion and the release of cytokine in the body of the subject, and the T cell response in the subject is enhanced as compared to T cells that include the CAR binding the solid tumor but don't include the CAR binding the WBC antigen or a tag as shown, for example, in FIG. 22.
145. The method of embodiment 144, wherein the T cell expansion is measured based on an increase in copy number of CAR molecules in genomic DNA of the T cells.
146. The method of embodiment 144, wherein the amount of the cytokine is enhanced as compared to T cells that include the CAR binding the solid tumor but don't include the CAR binding the WBC antigen or the tag.
147. The method of embodiment 146, wherein the cytokine is IL-6 or IFNγ.
148. Any suitable preceding embodiments, wherein the modified cells or cells are lymphocytes such as T cells, NK cells, macrophages, or DCs.
149. The method of any suitable preceding embodiments, further comprising: administering an effective amount of GCSF or GMCSF to the subject before, after, or at the same time as administering to the subject the effective amount of the population of lymphocytes.
150. Any suitable preceding embodiments, wherein the cells comprising CAR comprises JAK-STAT (e.g., JAK motif, STAT3, STATS motifs) enhance TIL, TCR cells, and/or expansion of CAR T cells in the subject without killing the WBC.
151. A method of enhancing cell expansion or expanding modified cells, the method comprising: introducing a polynucleotide encoding a binding molecule binding a solid tumor antigen into a cell to obtain a modified cell; obtaining a PBM (e.g., polyspecific antibody) binding a WBC antigen and a T cell antigen; and contacting the modified cell and the PBM (e.g., polyspecific antibody) with peripheral blood or B cells; allowing the modified cell to expand; wherein the expansion of the modified cell is greater than the expansion of a modified cell that is contacted with the peripheral blood but without the PBM (e.g., polyspecific antibody).
152. A method of enhancing cell expansion or expanding modified cells, the method comprising: obtaining a modified cell comprising a binding molecule binding a solid tumor antigen; obtaining a PBM (e.g., polyspecific antibody) binding a WBC antigen and a T cell antigen; and contacting the modified cell and the PBM (e.g., polyspecific antibody) with peripheral blood or B cells; allowing the modified cell to expand; wherein the expansion of the modified cell is greater than the expansion of a modified cell that is contacted with the peripheral blood but without the PBM (e.g., polyspecific antibody).
153. A method of enhancing cell expansion or expanding modified cells, the method comprising: administering an effective amount of a population of modified cells comprising a binding molecule binding a solid tumor antigen to a subject; and administering an effective amount of a PBM (e.g., polyspecific antibody) binding a WBC antigen and a T cell antigen, wherein the expansion of the population of modified cells is greater than the expansion of a population modified cells that are administered into a subject that is not administered with the PBM (e.g., polyspecific antibody).
154. Use of a PBM (e.g., polyspecific antibody) and a population of modified cells comprising: administering an effective amount of a population of modified cells comprising a binding molecule binding a solid tumor antigen to a subject; and administering an effective amount of a PBM (e.g., polyspecific antibody) binding a WBC antigen and a T cell antigen, wherein the expansion of the population of modified cells is greater than the expansion of a population modified cells that are administered into a subject that is not administered with the PBM (e.g., polyspecific antibody).
155. A pharmaceutical composition comprising a population of modified cells and PBMs (e.g., polyspecific antibodies) binding a WBC and a T cell antigen.
156. The method, use, or pharmaceutical composition of any suitable preceding embodiments, wherein the binding molecule is a CAR or TCR.
157. The method, use, or pharmaceutical composition of any suitable preceding embodiments, wherein the modified cell is a CAR T cell, TIL, or a TCR T cell.
158. The method, use, or pharmaceutical composition of any suitable preceding embodiments, wherein the modified cell is a T cell, NK, DC, or macrophage.
159. The method, use, or pharmaceutical composition of any suitable preceding embodiments, wherein the solid tumor antigen comprises a solid tumor antigen listed in this application and in PCT Patent Applications Nos: PCT/CN2016/075061, PCT/CN2018/08891, and PCT/US19/13068, all of which are incorporated herein by reference in their entirety.
160. The method, use, or pharmaceutical composition of any suitable preceding embodiments, wherein the WBC antigen is a B cell antigen listed in this application and PCT Patent Applications Nos: PCT/CN2016/075061, PCT/CN2018/08891, and PCT/US19/13068, all of which are incorporated herein by reference in their entirety.
161. The method, use, of pharmaceutical composition of any suitable preceding embodiments, wherein the PBM (e.g., polyspecific antibody) is a bispecific antibody or BITE® comprising an scFv binding CD3 and an scFv binding CD19, examples of the bispecific antibody or BITE® include Blinatumomab. Additional information regarding Blinatumomab and other BiTEs can be found at Topp et al., Long-term follow-up of hematologic relapse-free survival in a phase 2 study of blinatumomab in patients with MRD in B-lineage ALL. Blood 2012; 120 (26): 5185-5187, which is incorporated herein by its reference.
162. The method, use, or pharmaceutical composition of any suitable preceding embodiments, wherein the PBM (e.g., polyspecific antibody) comprises a PBM (e.g., polyspecific antibody) described in this application.
163. The method, use, or pharmaceutical composition of any suitable preceding embodiments, wherein the modified cell comprises a modified cell described in this application.
164. A composition comprising B cells and a bispecific antibody for use in a method of enhancing activation of modified cells, the bispecific antibody comprising a first binding domain binding CD3 and a second binding domain binding CD19, CD20, CD22, or BCMA, and the method comprising: obtaining modified cells comprising chimeric antigen receptor (CAR) T cells, wherein the CAR of the CAR T cells comprises a binding domain, a transmembrane domain, and an intracellular domain, the binding domain binding a solid tumor antigen; and contacting the CAR T cells with the composition comprising B cells and the bispecific antibody, thereby activating the modified cells, wherein level of activation of the CAR T cells is higher than level of activation in CAR T cells that are contacted with B cells without the bispecific antibody.
165. The composition for use of embodiment 164, wherein the level of activation is measured based on a level of expression of CD69, CD25, or CD137 in the modified cells.
166. A composition comprising polyspecific binding molecule (PBM) and a population of cells comprising an antigen of white blood cells (WBCs) for use in a method of expanding and/or activating modified cells, the PBM comprising at least a first binding domain binding a T cell and at least a second binding domain binding an antigen of a WBC, the method comprising: obtaining modified cells comprising a binding molecule that binds a solid tumor antigen; contacting the modified cells with the composition comprising the PBM and the population of cells comprising an antigen of WBC; and allowing the modified cells to expand and/or to be activated.
167. The composition for use of embodiment 166, wherein a level of expansion and/or activation in the modified cells is higher than a level of expansion and/or activation in modified cells that are contacted with the population of cells without the PBM.
168. The composition for use of any one of embodiments 1-4, wherein the solid tumor antigen comprises tMUC1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, 1L13-Rα2, Mesothelin, PSMA, ROR1, VEGFR-II, GD2, FR-α, ErbB2, EpCAM, EGFRvIII, B7-H3, CLDN18.2, MAGE A4, MSLN, CD205, or EGFR.
169. The composition for use of any one of embodiments 166-168, wherein the WBCs comprise a granulocyte, a monocyte, or a lymphocyte.
170. The composition for use of any one of embodiments 166-169, wherein the antigen of the WBC comprises CD19, CD22, CD20, BCMA, CD5, CD7, CD2, CD16, CD56, CD30, CD14, CD68, CD11b, CD18, CD169, CD1c, CD33, CD38, CD138, CD205, CD79a, CD79b, or CD13.
171. The composition for use of any one of embodiments 166-170, wherein the binding molecule is a CAR or a T cell receptor (TCR).
172. The composition for use of any one of embodiments 164-171, wherein the CAR comprises an antigen binding domain, a transmembrane domain, a co-stimulatory domain, and a CD3 zeta domain.
173. The composition for use of embodiment 172, wherein the co-stimulatory domain comprises the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof.
174. The composition for use of embodiment 171, wherein the TCR binds CEA, gp100, MART-1, p53, MAGE-A3, or NY-ESO-1.
175. The composition for use of any one of embodiments 166-174, wherein the modified cells are T cells, NK cells, macrophages, or dendritic cells.
176. The composition for use of any one of embodiments 166-175, wherein the activation is measured based on a level of expression of CD69, CD25, or CD137 in the modified cells.
177. The composition for use of any one of embodiments 166-176, wherein the expansion is measured based on numbers of modified cells or copy numbers of DNA encoding the CAR.
178. The composition for use of any one of embodiments 164-177, wherein the modified cells comprise an exogenous polynucleotide encoding a therapeutic agent comprising IL-1P, IL-2, IL-4, IL-5, IL-6, IL-7, IL-8, IL-10, IL-12, IL-13, IL-15, IL-17, IL-1Ra, IL-2R, IFNγ, MIP-In, MIP-IP, MCP-1, TNFα, GM-CSF, GCSF, CXCL9, CXCL10, CXCR factors, VEGF, RANTES, EOTAXIN, EGF, HGF, FGF-P, CD40, CD40L, or ferritin.
179. The composition for use of any one of embodiments 164-178, wherein the modified cells comprise a dominant negative form of PD-1, cytotoxic T lymphocyte antigen-4 (CTLA-4), B- and T-lymphocyte attenuator (BTLA), T-cell immunoglobulin mucin-3 (TIM-3), lymphocyte-activation protein 3 (LAG-3), T-cell immunoreceptor with Ig and ITIM domains (TIGIT), leukocyte-associated immunoglobulin-like receptor 1 (LAIRD, natural killer cell receptor 2B4 (2B4), or CD160.
180. The composition for use of any one of embodiments 164-179, wherein the PBM or bispecific antibody comprises SEQ ID NO: 6.
181. A pharmaceutical composition comprising an effective amount of the modified cells obtained by using the composition in any one of embodiments 164-180.
182. Use of the composition of any one of embodiments 164-180 for enhancing activation or expanding and/or activating modified cells comprising CAR T cells that bind a solid tumor antigen or comprising a binding molecule that binds a solid tumor antigen.
183. A kit or pharmaceutical composition comprising a modified cell comprising CAR T cells, wherein the CAR T cells comprise a binding domain, a transmembrane domain, and an intracellular domain, the binding domain binding a solid tumor antigen; and a bispecific antibody or a PBM.
184. The kit or pharmaceutical composition of embodiment 183, wherein the bispecific antibody comprises a first binding domain binding CD3 and a second binding domain binding CD19, CD20, CD22, or BCMA.
185. The kit or pharmaceutical composition of embodiment 184, wherein the PBM comprises at least a first binding domain binding a T cell and at least a second binding domain binding an antigen of a white blood cells (WBC).

### EXAMPLES

### Example 1: CAR T Cell and T Cell Expressing Antigen

Lentiviral vectors that encode individual CAR molecules were generated and transfected into T cells, which are explained below. Techniques related to cell cultures and cytotoxic T lymphocyte assays can be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8, 1453-1464, which are incorporated herein by reference in its entirety.

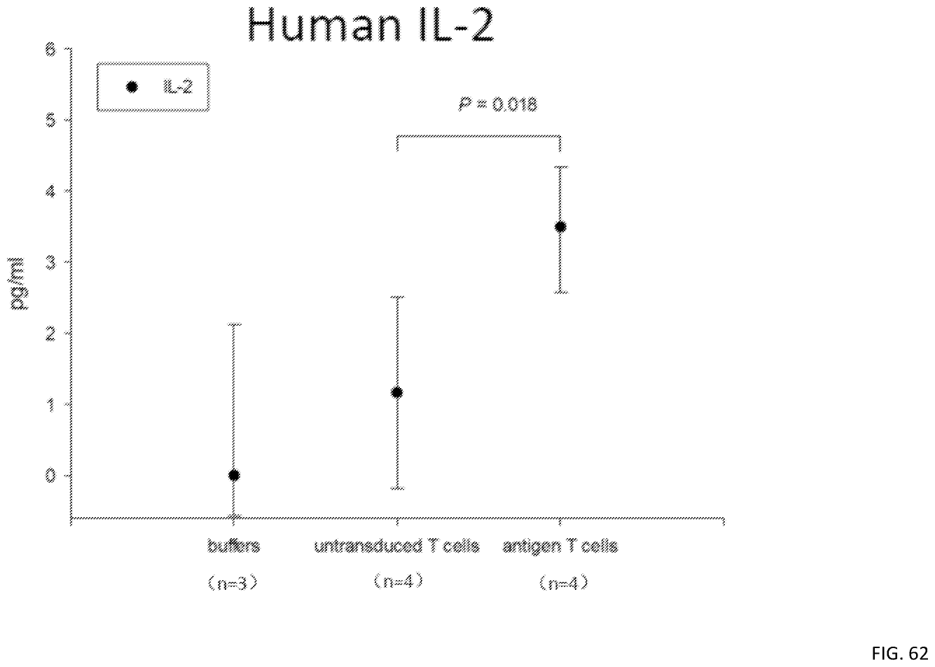

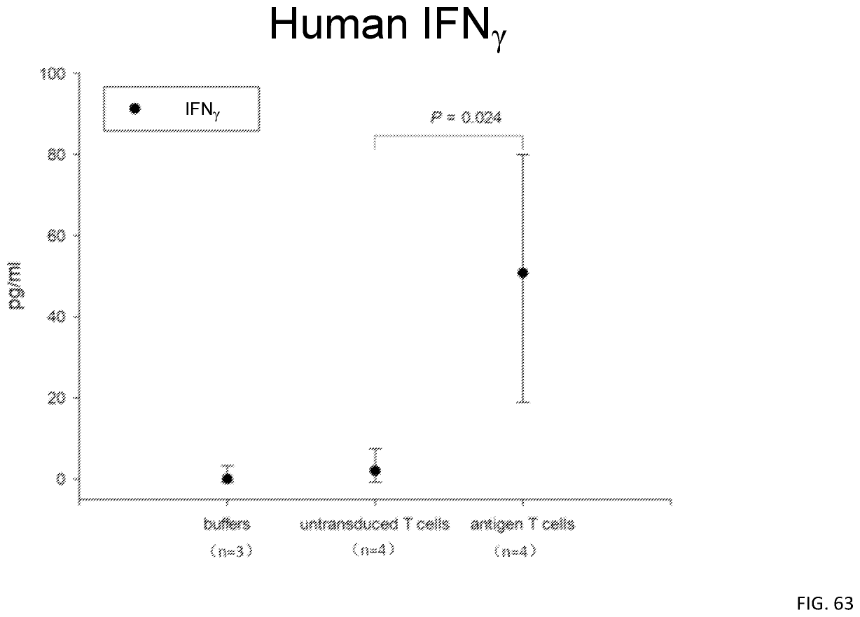

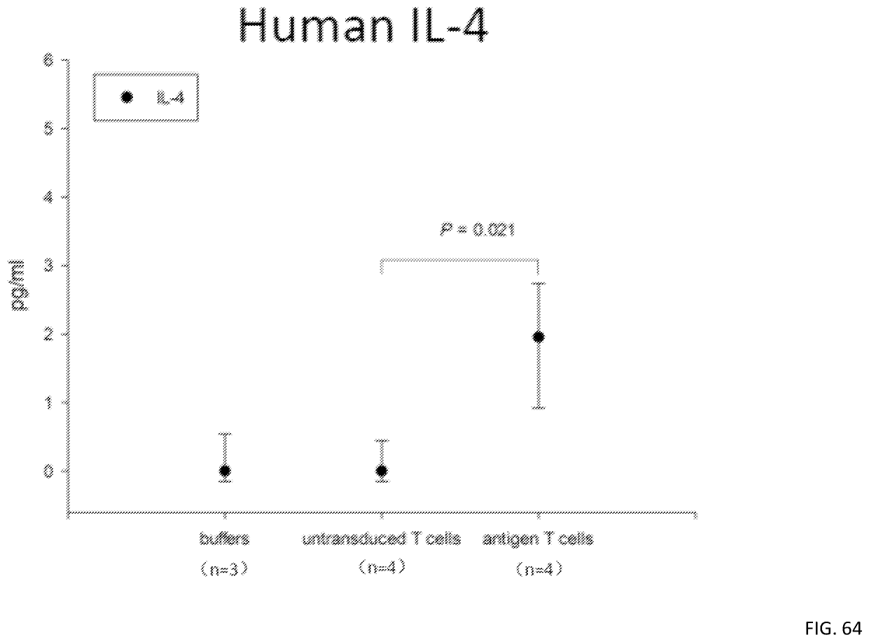

Primary T cells were obtained from volunteers and divided into several groups. One group of T cells was transduced with lentiviral vectors encoding anti-TSHR CAR to generate anti-TSHR CAR T cells. One group of T cells was transduced with lentiviral vectors encoding TSHR to generate TSHR-overexpressed T cells. NOD scid gamma (NSG) mice were divided into three groups: Experimental Group, Group 1, and Group 2, as shown in Table 4. NSG mice were irradiated, and a specific number of anti-TSHR CAR T cells and corresponding cells were infused into different groups of mice, respectively. For the Experimental Group, the mice were infused with anti-TSHR CAR T cells and TSHR-overexpressed T cells. For Control Group 1, the mice were infused with anti-TSHR CAR T cells and primary T cells. For Control Group 2, the mice were infused with anti-TSHR CAR T cells and buffer. After the infusions were completed, blood from the limbal vein of the mice was collected to analyze T cell expansion and cytokines (e.g., IFNγ, IL4, IL2) released in the peripheral blood of the mice. The mice were then sacrificed and further analyzed to collect data. As shown in FIGS. 62-64, the amount of cytokine released in the Experimental Group was greater than those in the Control Groups. These results demonstrate that infusion of cells expressing an antigen enhances the response of the corresponding CAR T cells. The schedule for in vivo analysis is provided in Table 5 below.

TABLE 4

| Experimental Group | Control Group 1 | Control Group 2 |
|---|---|---|
| Anti-TSHR CAR T cells about | Anti-TSHR CAR T cells about | Anti-TSHR CAR T cells about |
| 4 × 10⁶/mouse | 4 × 10⁶/mouse | 4 × 10⁶/mouse |
| Antigen T (TSHR- | NT (non-transduced T cell) | N/A |
| overexpressed T cell) about | about 4 × 10⁶/mouse per time | |
| 4 × 10⁶/mouse per time | | |

TABLE 5

| Day 1 | Day 3 | Day 5 | Day 9 | Day 12 | Day 14 | Day 21 | Day 28 |
|---|---|---|---|---|---|---|---|
| irradiation | CAR T | T cells or | T cells or | T cells or | bleeding | bleeding | sacrifice |
| at | infusion | buffers | buffers | buffers | and | and | and |
| 1.5 Gy | | infusion | infusion | infusion | analysis | analysis | analysis |

The treatment methods described herein can easily be adapted for other species or subjects, such as humans.

### Example 2: Signaling Involved in CoupledCAR®

Mixed cells including CD19 CART cells and GCC CAR T cells were prepared and cultured with B cell (Group 1) and K562 cells (Group 2), respectively. It was found that GCC CAR T cells were activated and expanded in the presence of B cells or K562 cells. Further, the activation and expansion were enhanced when GCC CAR T cells were prepared using CD3/CD28 agonists (e.g., TransAct™). Further, it was found that more cytokine release was released in the mixed cells in Group 2 than in Group 1. Flow cytometry assay was conducted to determine the expression of B cells and K562 cells after being cultured with the mixed cells. It was found that the expression of CD80/CD86 in K562 cells was higher than that in B cells, indicating K562 cells showed more co-stimulatory effect than that of B cells. Also, a flow cytometry assay was conducted on the mixed cells. It was found that the expression of CD25 in Group 2 is higher than in Group 1. CD25 is a receptor of IL-2. It has been reported that CD80 is critical for high level CD25 expression in T cells. Thus, these results indicate that the IL-2 signaling pathway may contribute to the activation and expansion of GCC CAR T cells in CoupledCAR®. Additional information regarding CoupledCAR® can be found in PCT Publication WO 2020146743, which is incorporated herein by reference in its entirety.

Further, it has been reported that IL-12 enhances T cell response. To understand the potential mechanisms, GCC CAR T cells were generated, activated, and then cultured with media containing IL-12. Flow cytometry was conducted to analyze the expression of markers on GCC CAR T cells. It was found that GCC CAR T cells up-regulated expression of CD40L, and such up-regulation was enhanced after IL-12 was added to the media. It has been reported that CD40-CD40L was the key to the activation of monocytes, macrophages, DC, and B cells. Thus, these results indicate that the CD40-CD40L signaling pathway on T cells may contribute to CAR T activation and expansion in vivo.

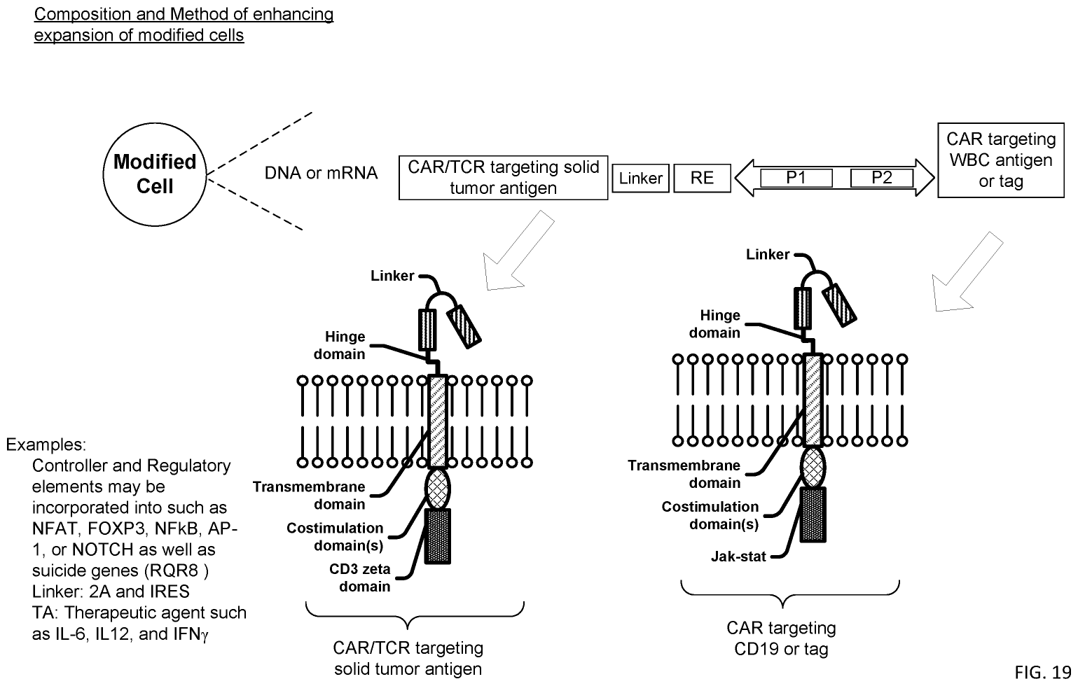

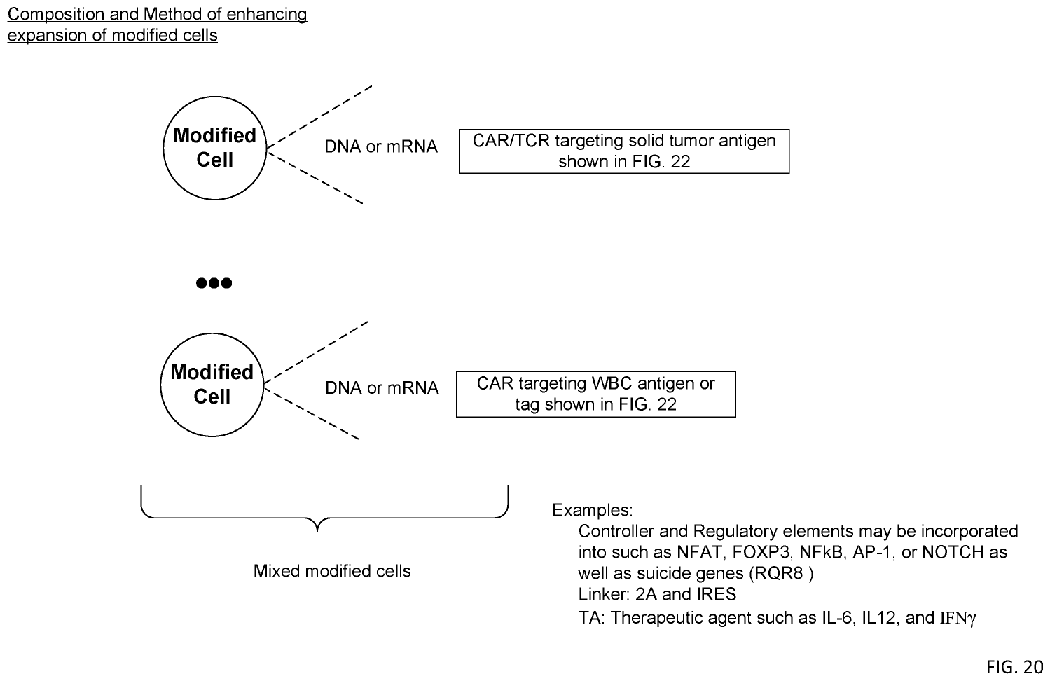

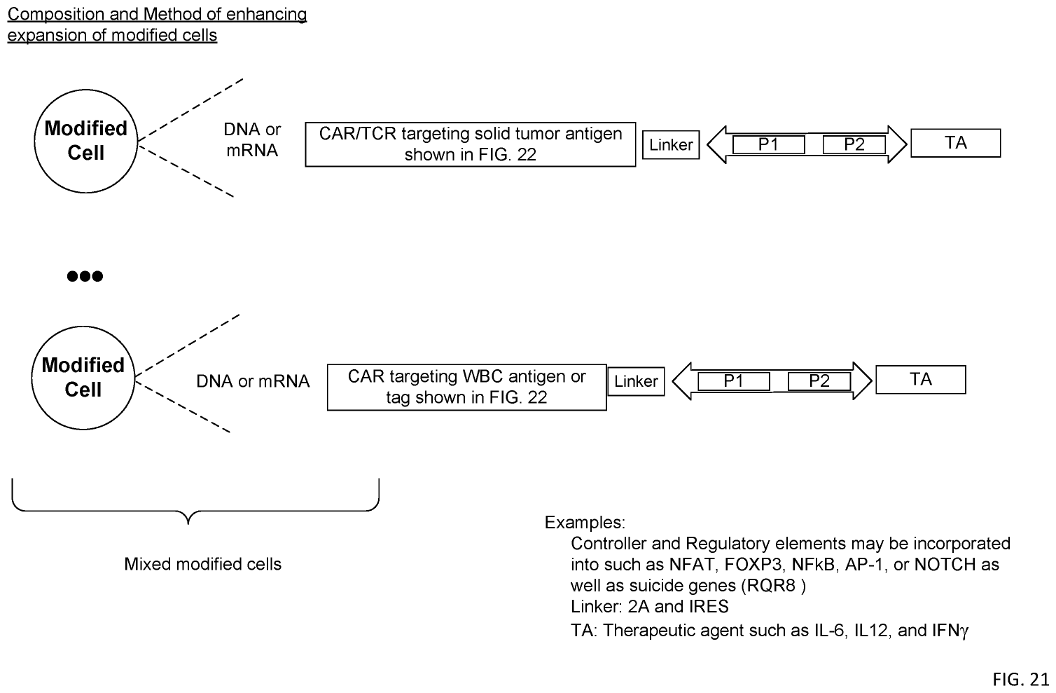

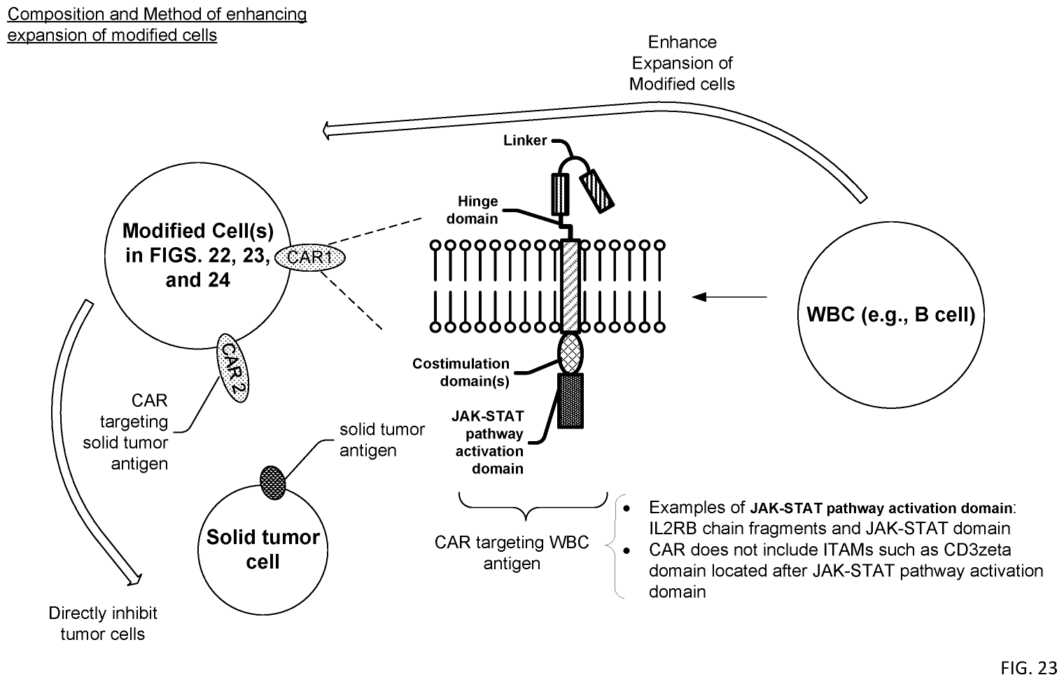

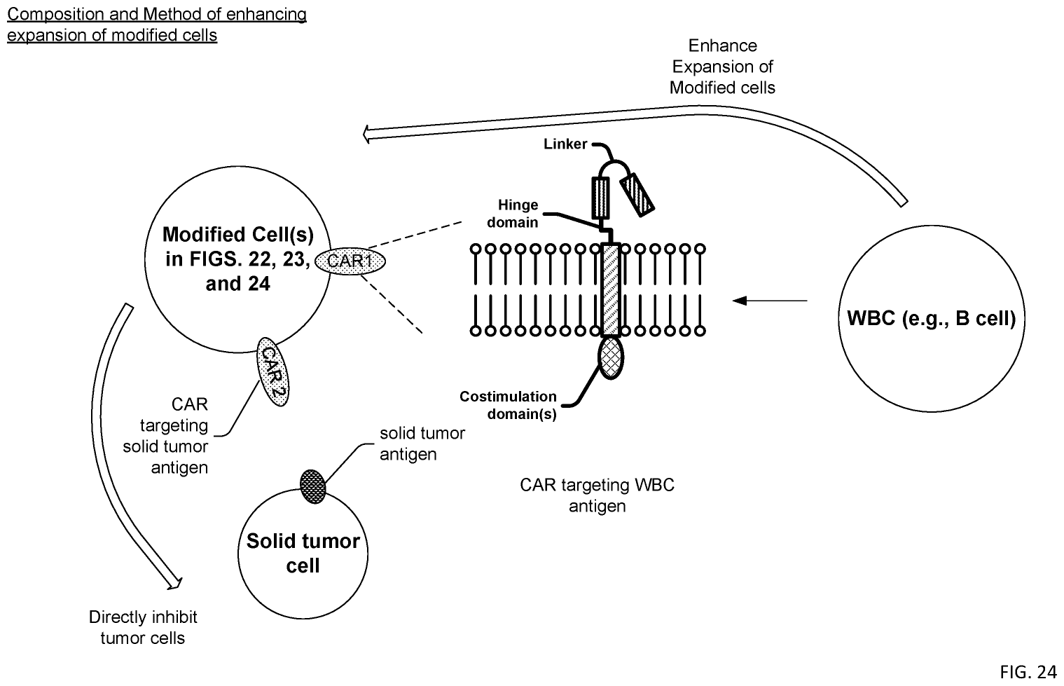

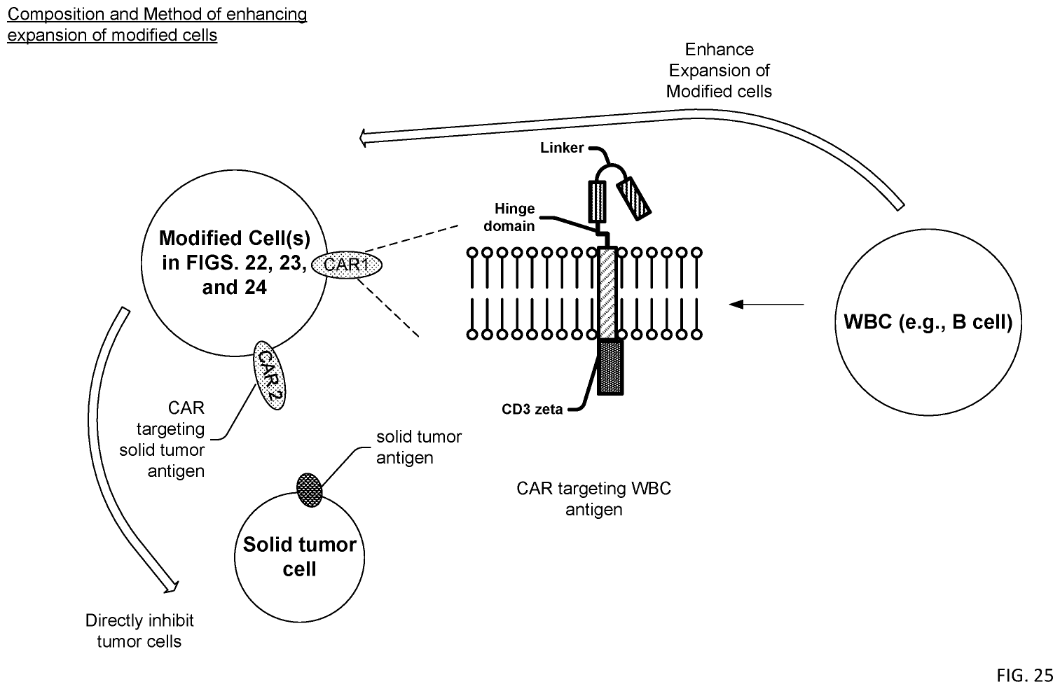

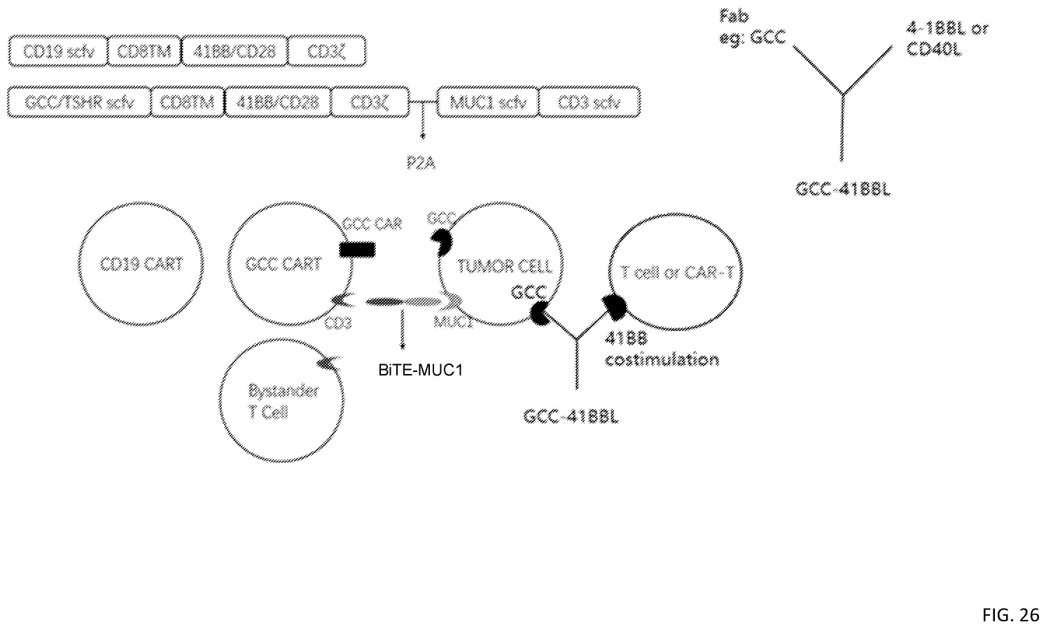

Accordingly, IL-2 and CD40-CD40L signaling pathways on T cells are implemented to enhance T cell response in vivo (e.g., expansion and activation) with fewer side effects (e.g., killing of B cells) as compared to the CoupledCAR® that includes CD19 CAR T cells. FIG. 19-26 show various designs that enhance cell expansion in vivo. For example, as shown in FIG. 22, a subject is administered with a composition comprising Tag-Anti-CD40; CD40+ cells of the subject are then activated, and these CD40+ cells may activate and/or expand T cells or modified T cells. As shown in FIG. 23, CAR 1 recognizes CD19, activates the CD28 signaling pathway, and activates the CD80/CD86 signaling pathway, which is downstream of the JAK-STAT signaling pathway. Activation of these signaling pathways will cause the modified cells to expand, but due to the absence of the CD3 zeta domain, it will not kill the WBCs.

### Example 3: Use of Polyspecific Binding Molecules (PBMs) in CoupledCAR®

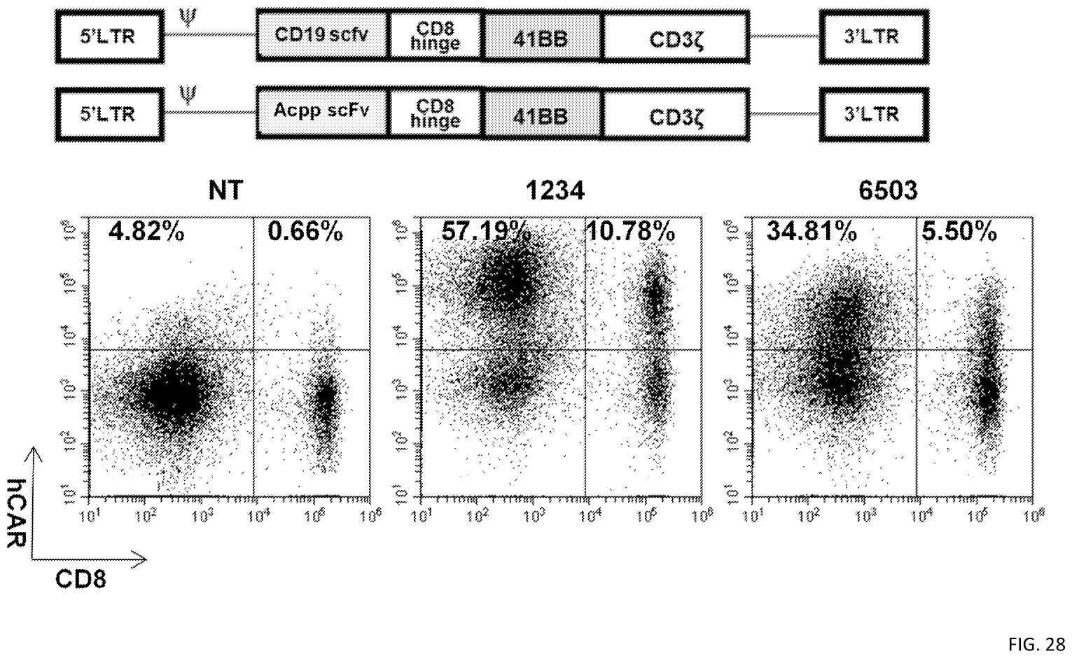

Peripheral blood was drawn from healthy volunteers. CD3+ T cells from the peripheral blood were sorted with pan T Kit. These T cells were cultured with media containing TransAct™. Two types of CAR T cells were then generated. Constructs of CARs as well as reference numbers for the cells containing the CAR construct are provided in Table 6. Flow cytometry assay was conducted to determine CAR ratios and cell phenotypes, which are shown in FIG. 28.

TABLE 6

| Name | Construction | Notes |
|---|---|---|
| 1234 | CAR-h19-bbz | Humanized CD19 scFv and 4-1BB |
| 6503 | CAR-ACPP-bbz | ACPP (or PAP) scFv and 4-1BB |

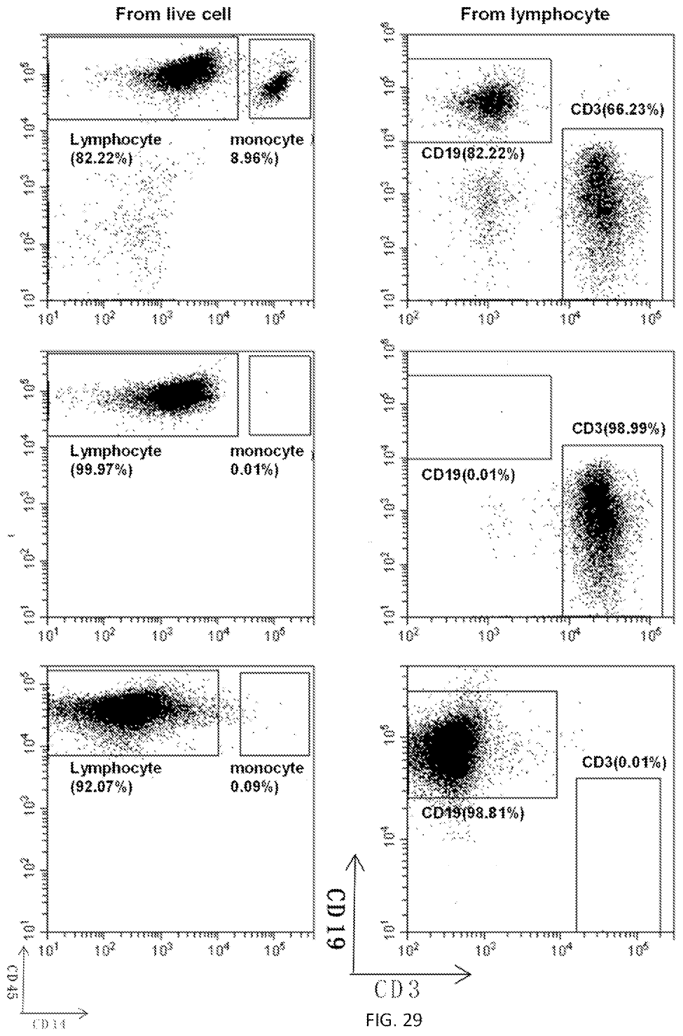

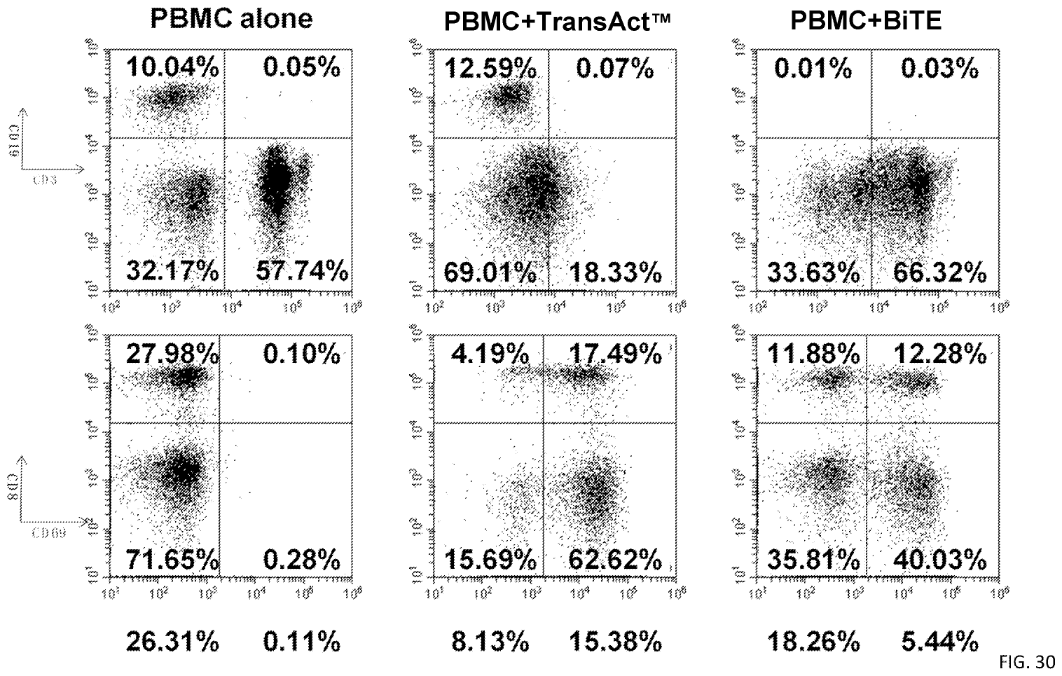

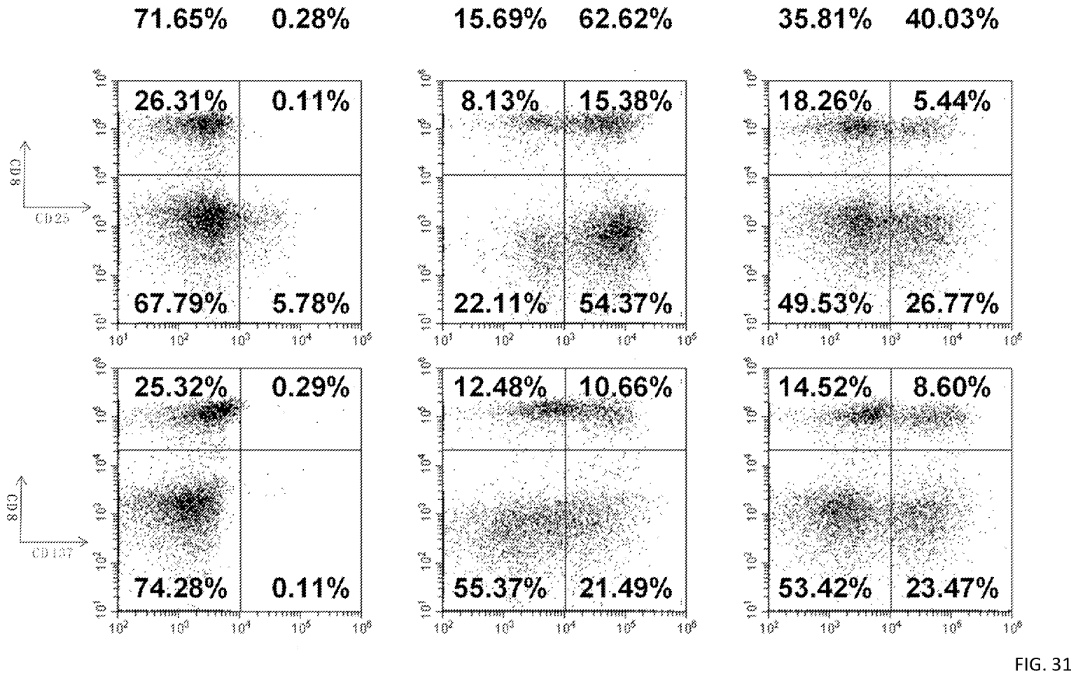

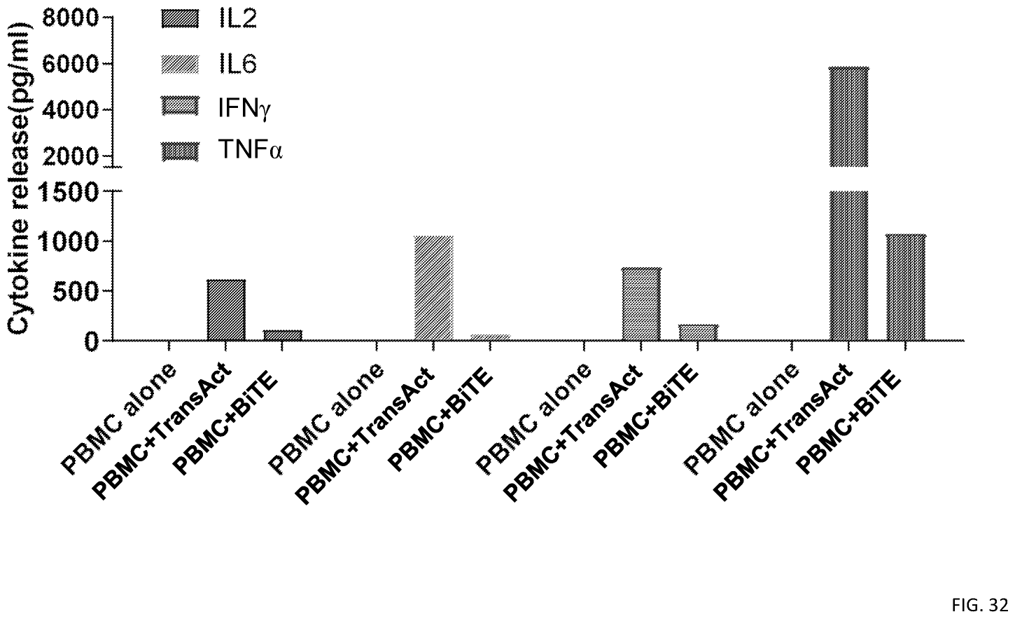

CD3-CD19 BiTEs (e.g., blinatumomab, Blincyto®) were generated based on the references such as Runcie et al. Molecular Medicine (2018) 24:50, and their functions in CoupledCAR® were further investigated. FIGS. 29-32 show that CD3-CD19 BiTE® can activate T cells in the presence of CD19+ cells, which these T cells do not bind. T cells and B cells were obtained from peripheral blood mononuclear cells (PBMCs) of volunteers. Three groups of cells were prepared. Group 1 was the negative control and included PBMCs cultured using media without TransAct™ and IL-2. Group 2 was the positive control and included PBMC cultured using media with TransAct™ and IL-2. Group 3 was the experimental group and included CD3-CD19 BiTEs and PBMCs cultured with TransAct™ and IL-2. After cells were cultured for 24 hours, a flow cytometry assay was conducted to determine the expression of activation markers CD69, CD137, and CD25 in these three groups. Also, B cells in Group 3 were not detected. FIG. 29 shows flow cytometry results of isolation of lymphocytes. FIG. 30 shows flow cytometry results of in vitro assay. FIG. 31 shows other flow cytometry results of the in vitro assay. FIG. 32 shows a summary of the in vitro assay shown in FIGS. 30 and 31. These results show that BiTEs enhance activation of T cells, induce cytokine release of T cells in the presence of B cells, and cause the B cells to be lysed by T cells in the PBMCs.

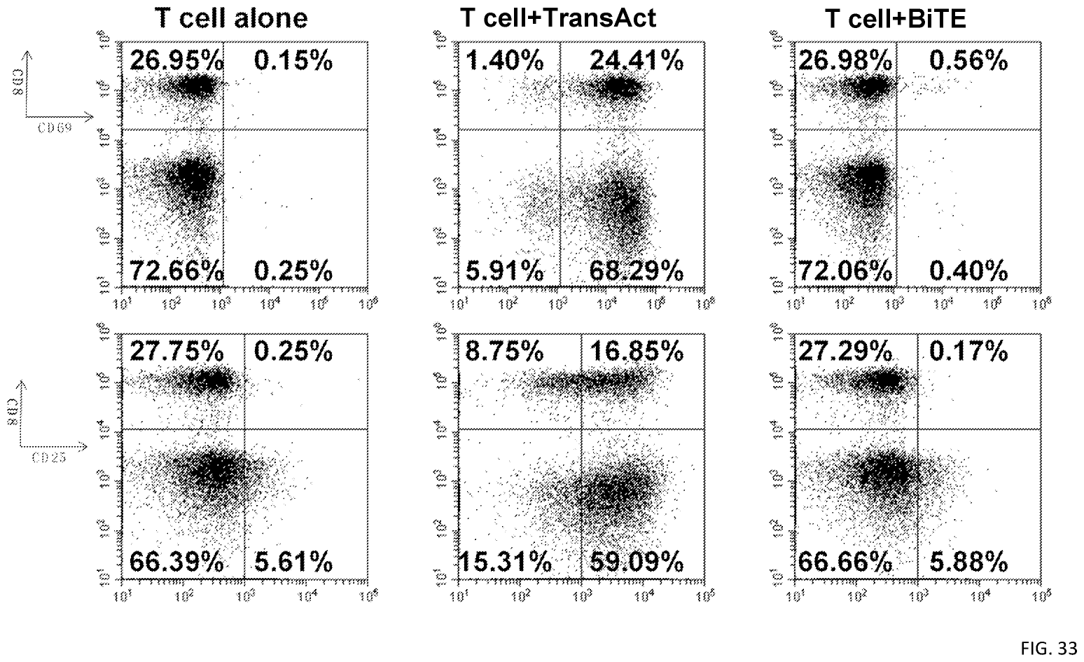

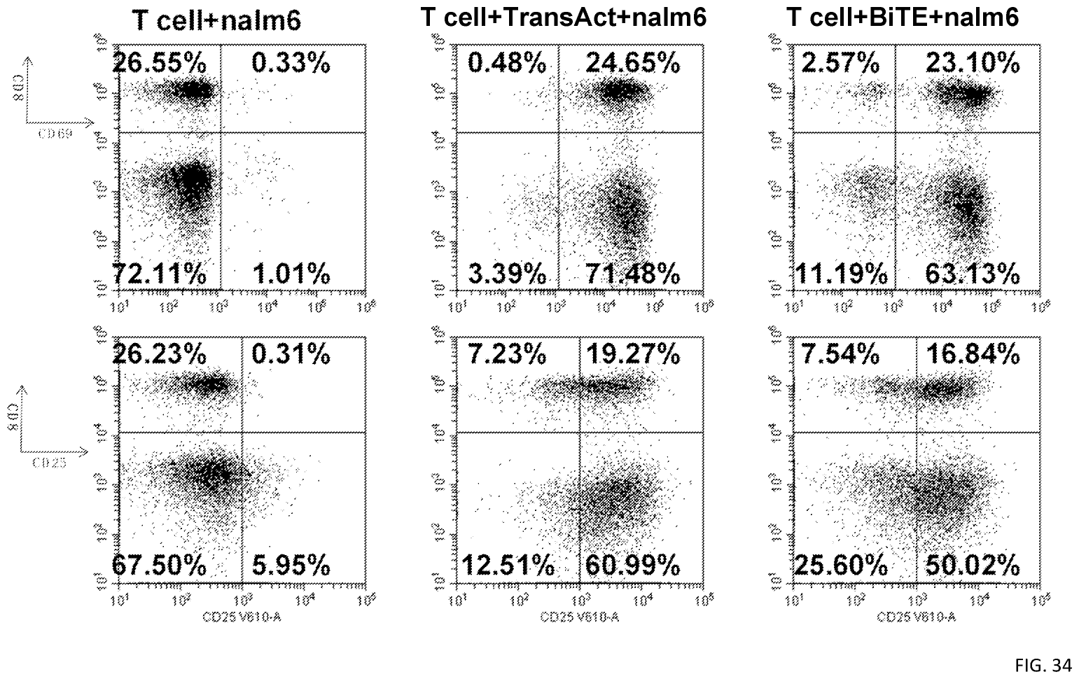

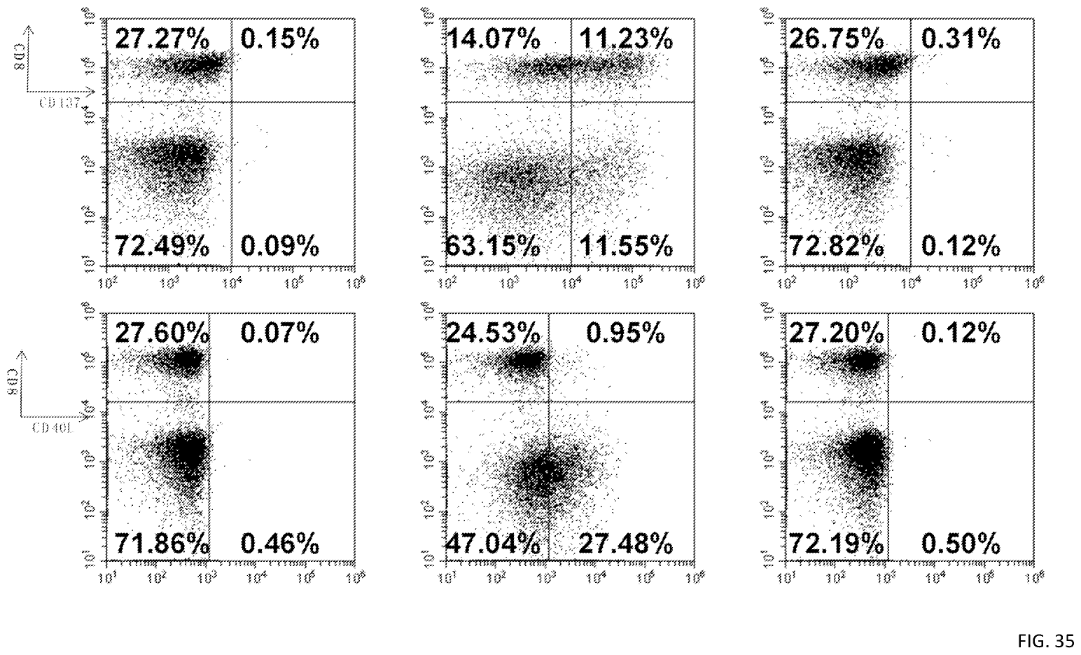

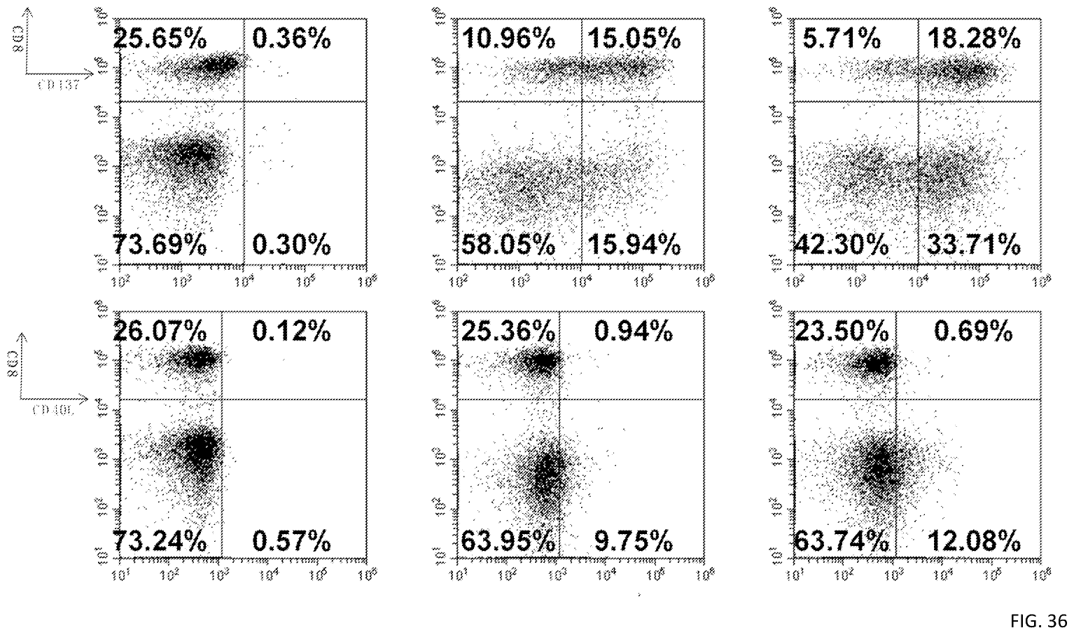

FIGS. 33-36 show results of in vitro assay that BiTEs can activate T cells in the presence of CD19+ Nalm6 cell line. In these experiments, Group 1 included T cells cultured using media without TransAct™ and IL-2; Group 2 included T cells cultured with TransAct™ and IL-2 without Nalm6 cells; and Group 3 included CD3-CD19 BiTEs and T cells cultured without TransAct™ and IL-2 but with Nalm6 cells. The results showed that T cells in Groups 3 and 2 showed significant activation based on increased expression of CD69, CD25, and CD137 of CD4 and CD8 subtype T cells. FIG. 33 shows flow cytometry results of in vitro assay. FIG. 34-36 shows additional flow cytometry results of the in vitro assay. These results show that CD3-CD19 BiTEs activate T cells in the presence of Nalm6 cells.

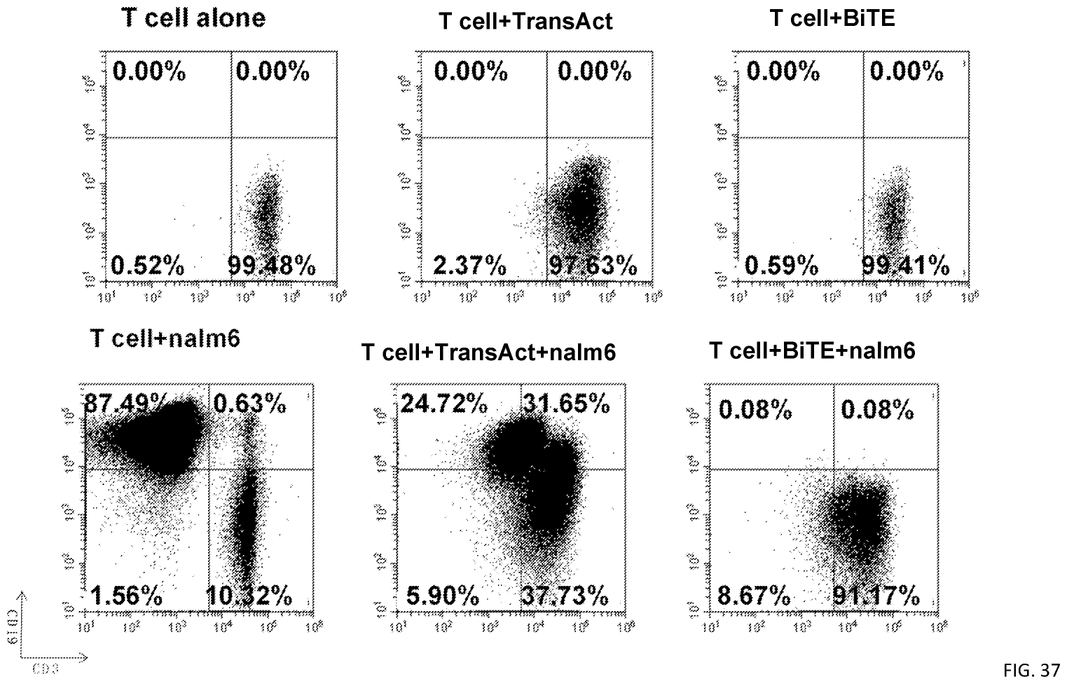

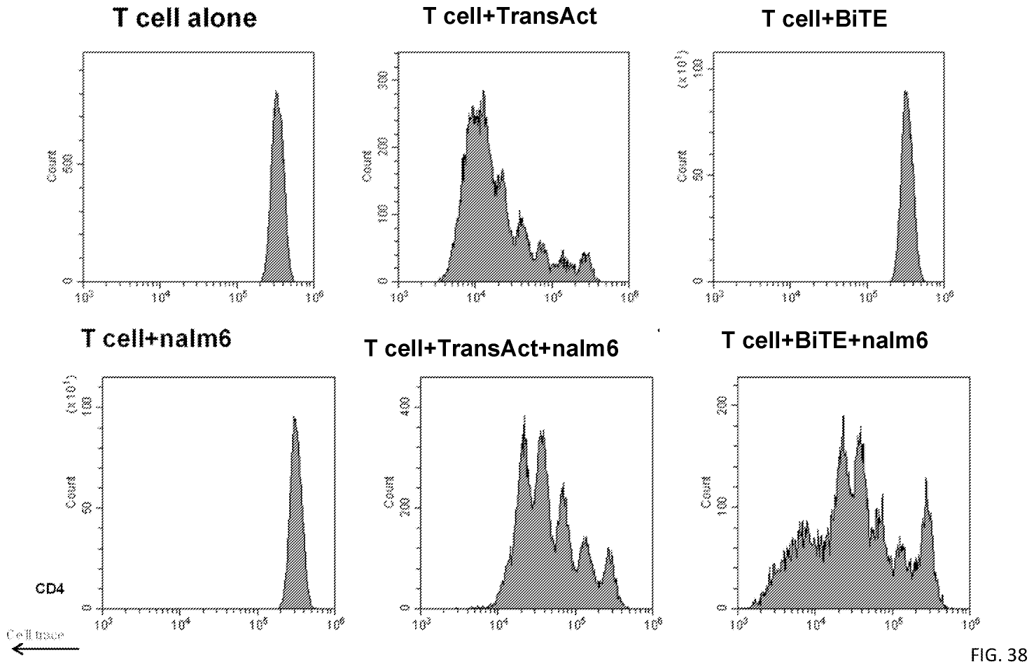

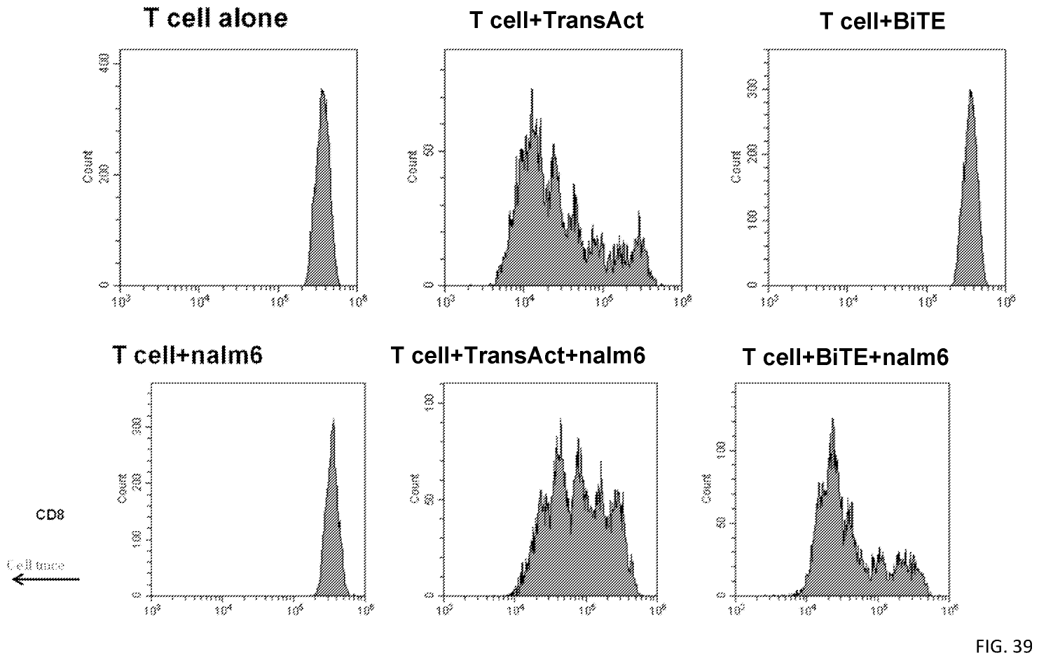

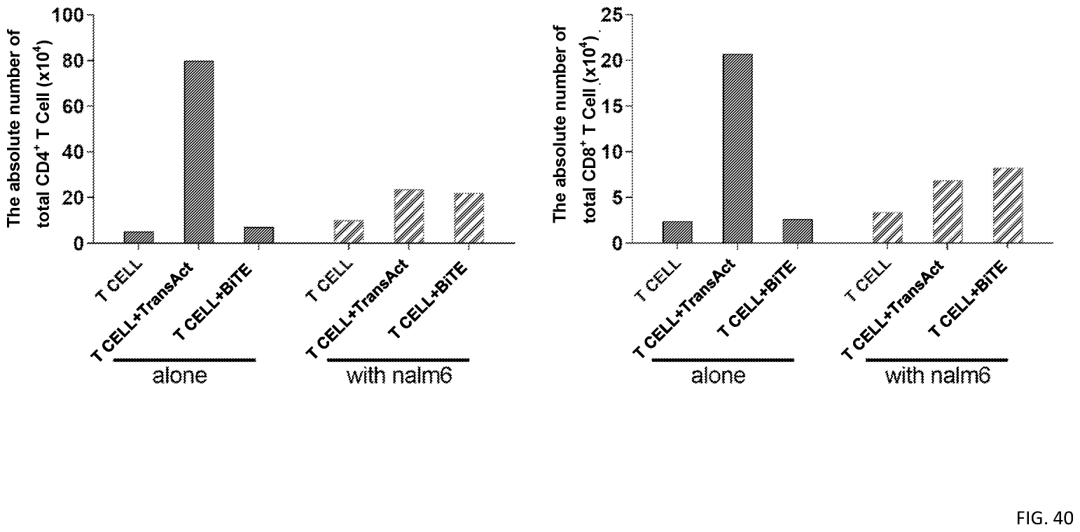

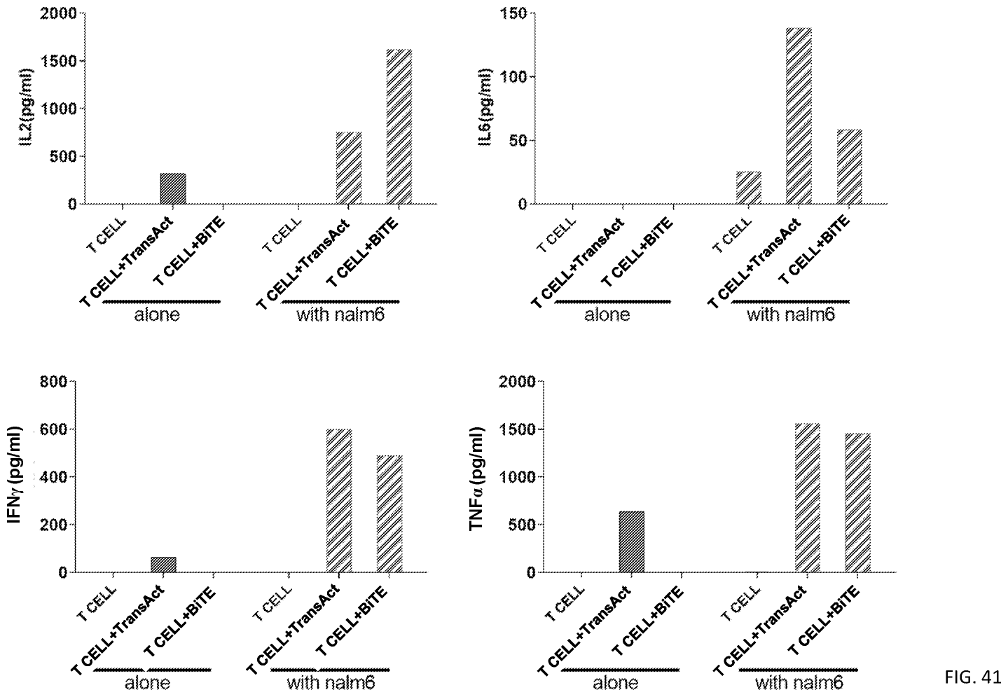

FIGS. 37-41 show results of in vitro assay that T cell can kill CD19+ Nalm6 cells in the presence of BiTEs and achieve similar expansion as in the presence of CD3/CD28 agonists (TransAct™). In these experiments, Group 1 included T cells cultured using media without TransAct™ and IL-2; Group 2 included T cells cultured with TransAct™ and IL-2; and Group 3 included CD3-CD19 BiTEs and T cells cultured with Nalm6 cells but without TransAct™ and IL-2. FIG. 37 shows flow cytometry results of in vitro assay. FIG. 38 shows cell expansion results of the in vitro assay. FIG. 39 shows other cell expansion results of the in vitro assay. FIG. 40 shows a summary of the in vitro assay shown in FIGS. 38 and 39. FIG. 41 shows the results of the cytokine release assay indicate that BiTEs enhanced cytokine release of T cells. After these cells were cultured for 24 hours, the supernatant was collected to detect the Cytometric Bead Array (CBA). It was found that Group 2 and Group 3, as compared to Group 1, show increased levels of cytokine release of IL-6, TNF-α, and IFNγ, which contributed to the inflammation environment to promote anti-tumor activities.

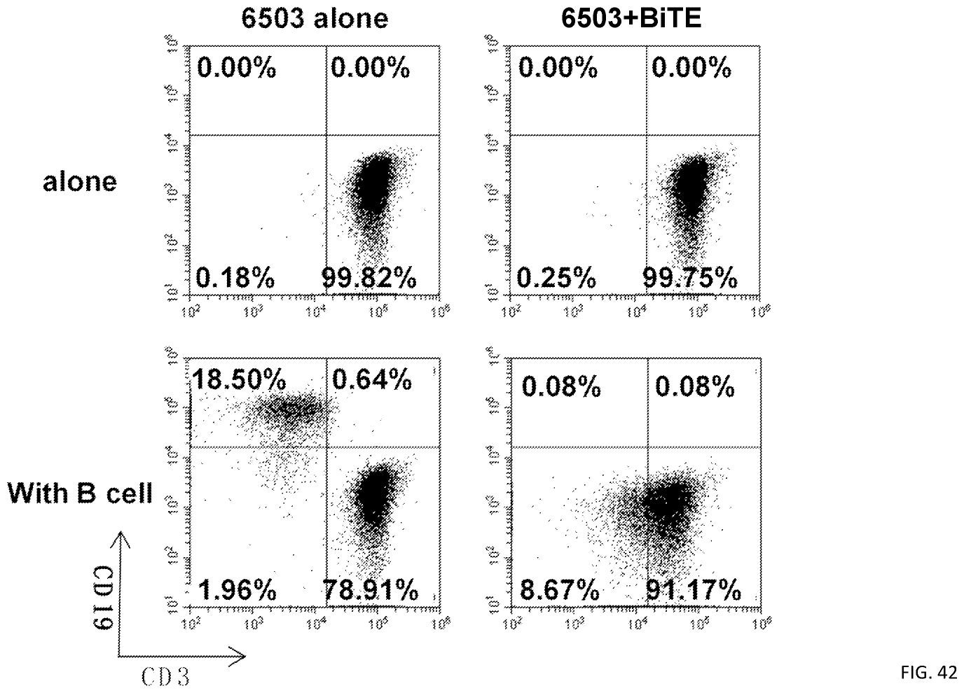

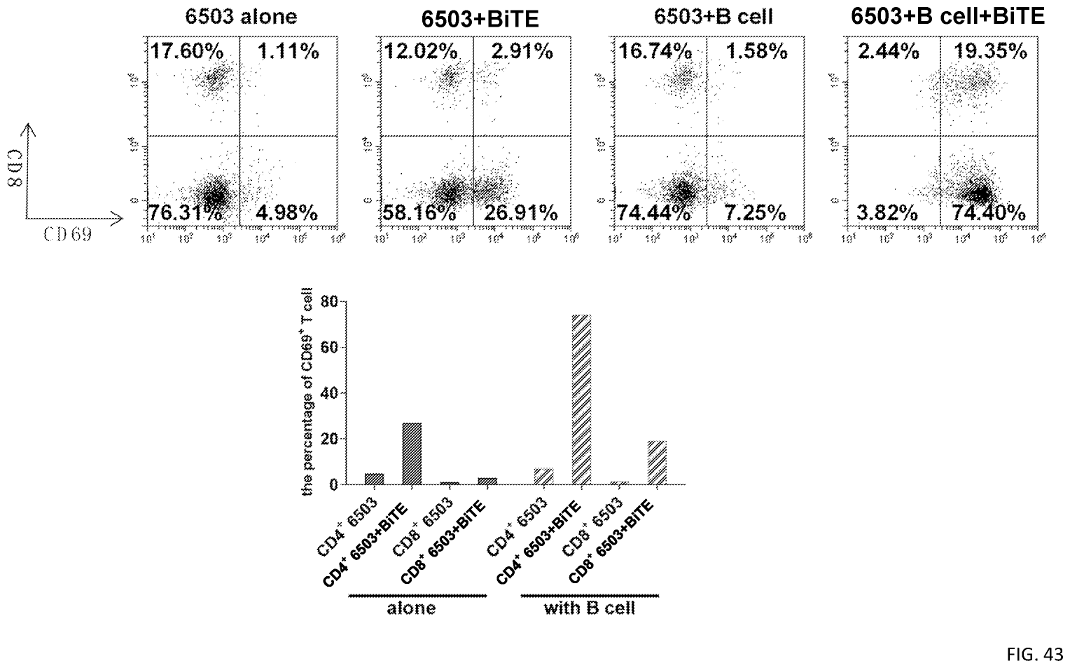

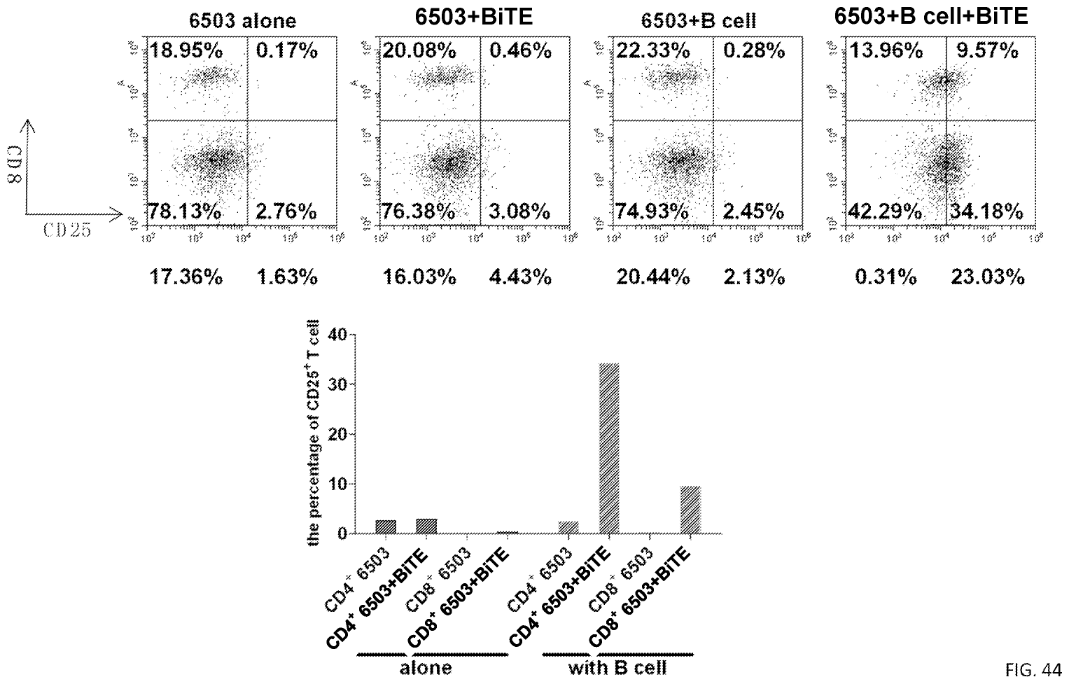

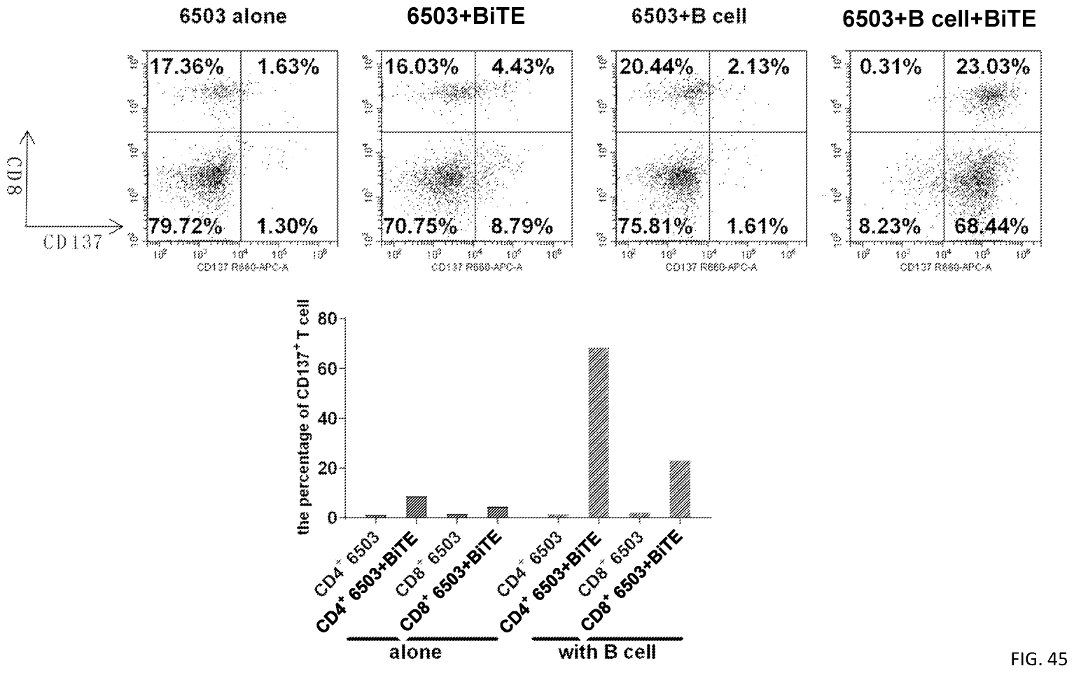

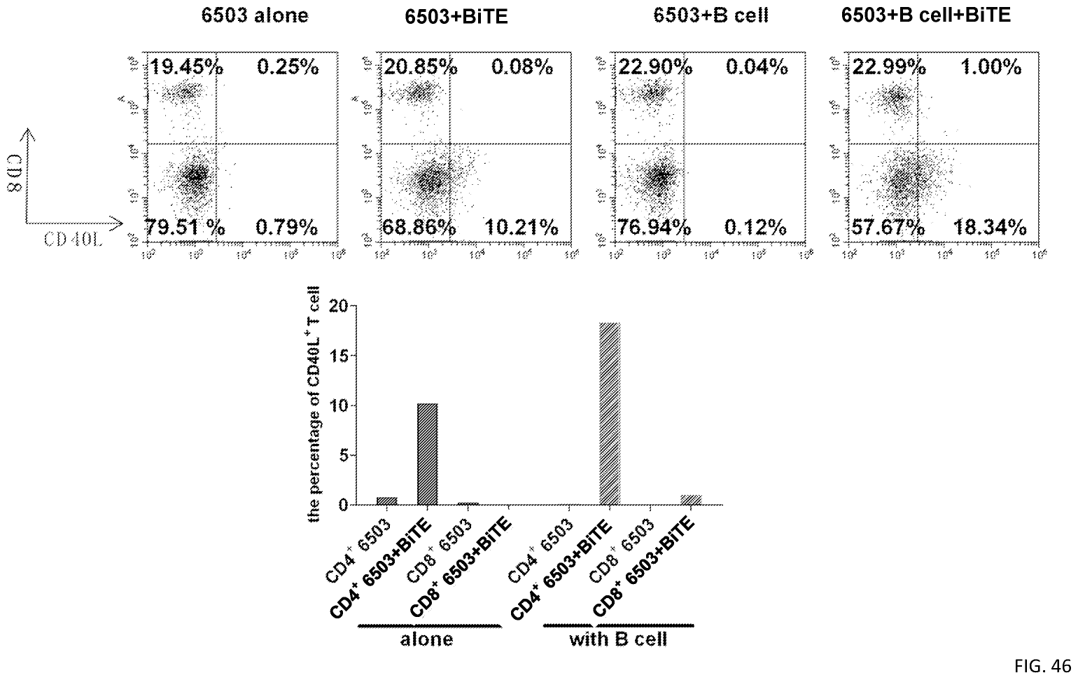

FIGS. 42-46 show flow cytometry results suggesting that BiTEs killed CD19+ cells and promoted activation of CAR T cells. In these experiments, Group 1 included PAP CAR T cells cultured using media without TransAct™ and IL-2; Group 2 included PAP CAR T cells cultured with TransAct™ and IL-2 without B cells; Group 3 included CD3-CD19 BiTEs and PAP CAR T cells cultured without TransAct™ and IL-2 but with B cells; and Group 4 included CD3-CD19 BiTEs and PAP CART cells cultured with TransAct™ and IL-2 as well as B cells. After the cells were cultured for 24 hours, a flow cytometry assay was conducted to detect cell surface markers. The results showed that cells in Group 1 and 3 were not well activated, which is different from the results shown in FIGS. 29-41. FIG. 42-46 shows flow cytometry results of in vitro assay. These results show CD3-CD19 BiTEs activated and expanded PAP CART cells in the presence of B cells. Further, CD40L expression of CD4 subtype T cells was up-regulated in Group 4, indicating that BiTEs can activate CAR T in the presence of B cells.

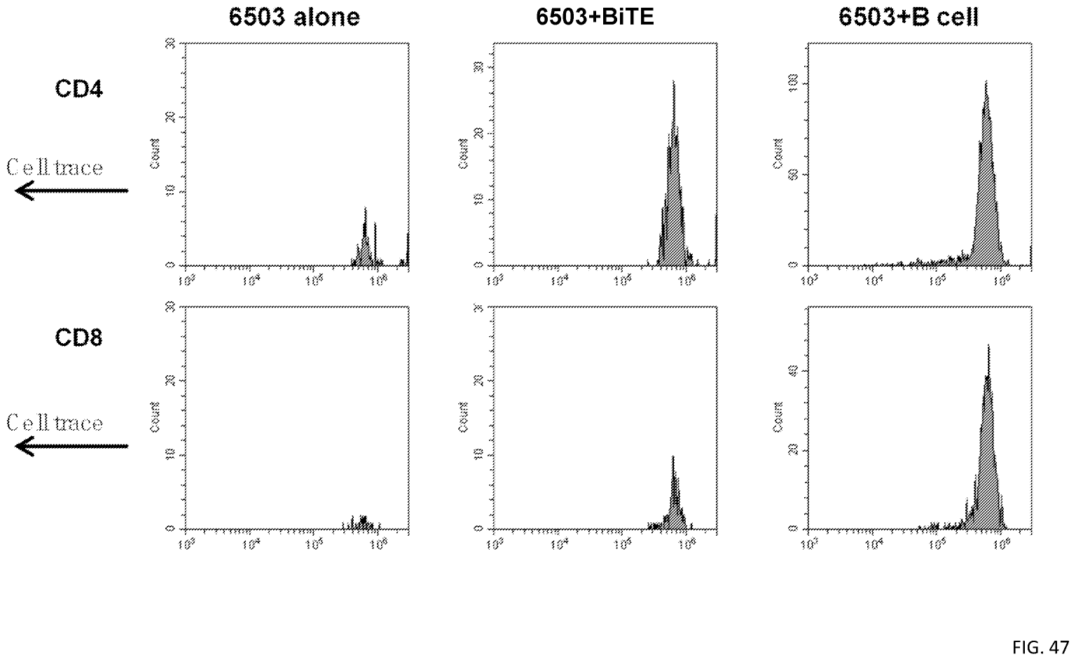

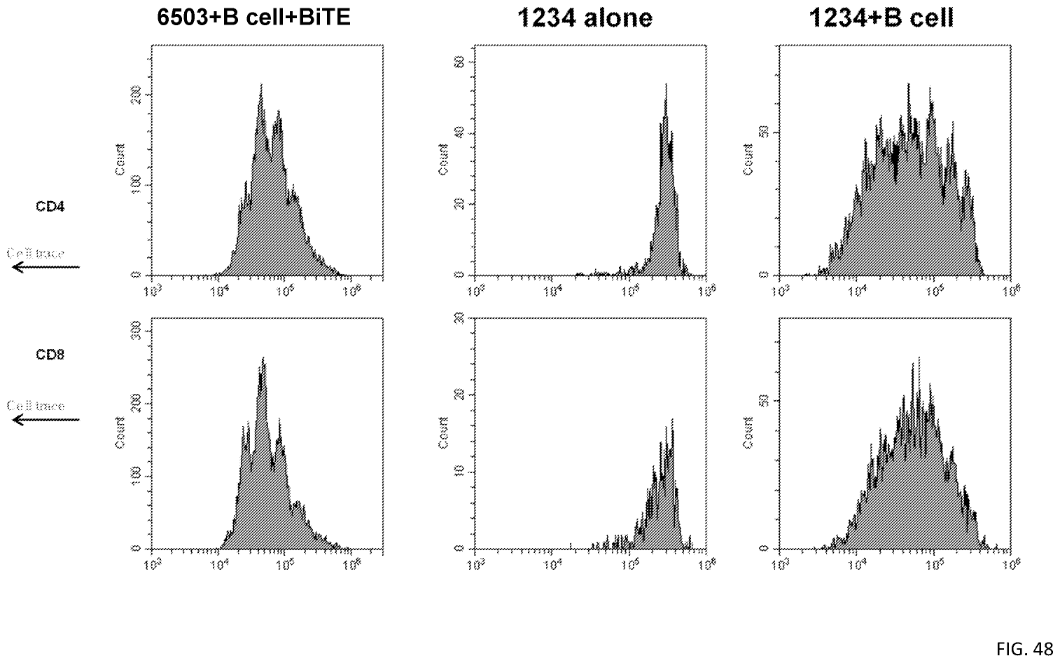

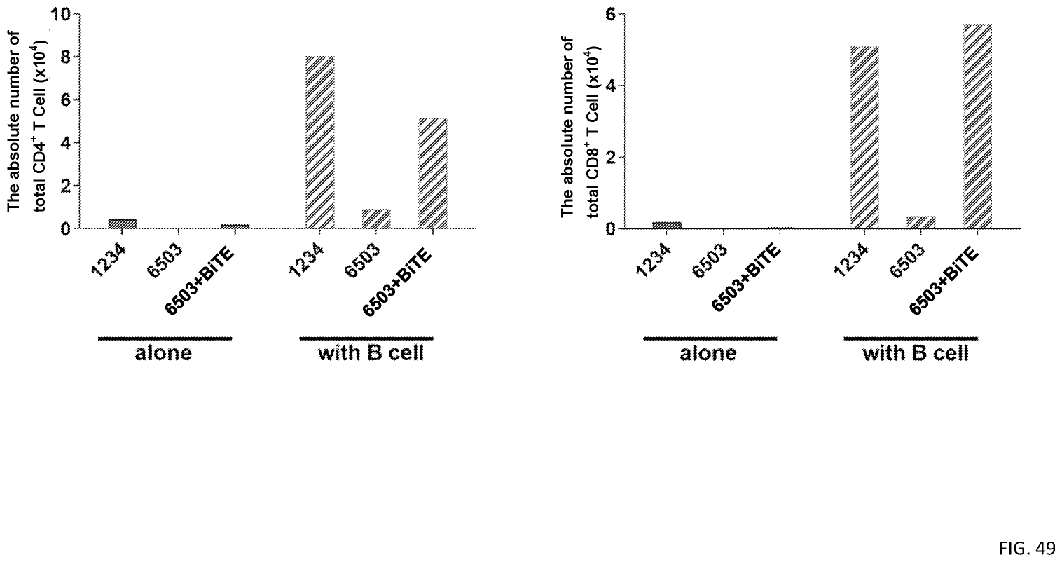

FIGS. 47-49 show cell expansion assay that BiTEs promoted the expansion of PAP CAR T cells in the presence of B cells. FIG. 47 shows cell expansion results of in vitro assay. FIG. 48 shows other cell expansion results of the in vitro assay. FIG. 49 shows a summary of the in vitro assay shown in FIGS. 47 and 48. In these experiments, Group 1 included PAP CAR T cells cultured without B cells; Group 2 included PAP CAR T cells cultured with B cells; Group 3 included PAP CART cells cultured with B cells and CD3-CD19 BiTEs; Group 4 included PAP CAR T cells cultured without B cells and CD3-CD19 BiTEs; and Group 5 included CD19 CAR T cells cultured with B cells as a positive control. Cells of the different groups were cultured for 120 hours, and a CellTrace™ assay was conducted to determine T cell expansion. It was found that PAP CAR T cultured with B cells and CD3-CD19 BiTEs showed similar results of positive control, indicating that BiTEs caused solid tumor CAR T cells to expand in the presence of B cells.

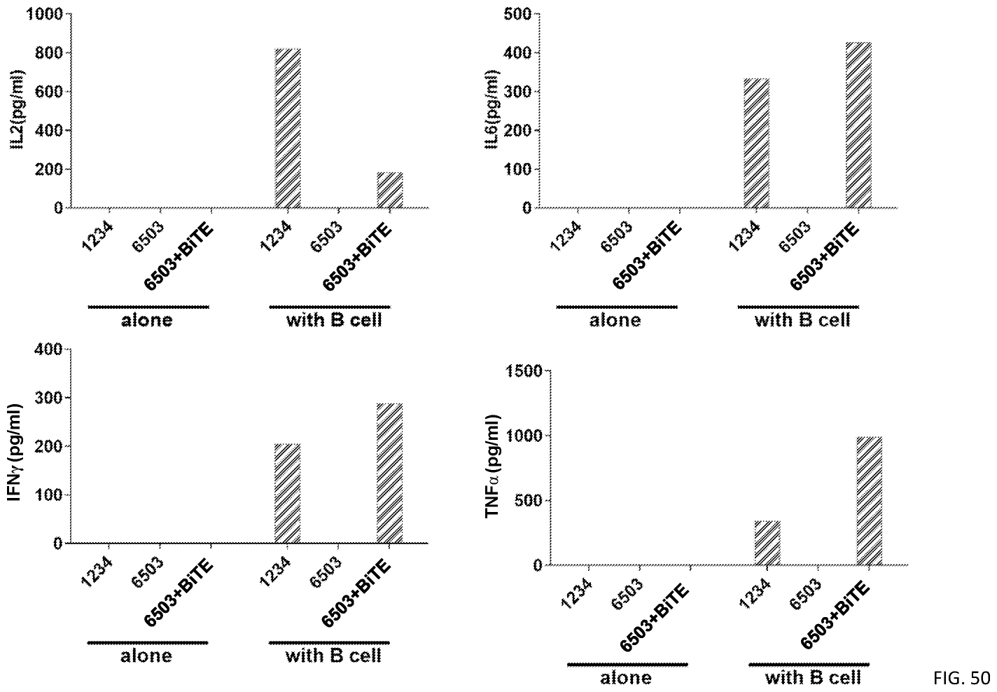

FIG. 50 shows results of cytokine release that BiTEs stimulated CAR T cells to release cytokines in the presence of CD19+ B cells. Various groups of cells were cultured for 24 hours, and the supernatant was collected for CBA. It was found that PAP CAR T cells cultured with B cells and CD3-CD19 BiTEs showed similar results of positive control, which is CD19 CAR T cells cultured with B cells, indicating that BiTEs caused solid tumor CAR T cells to release cytokines.

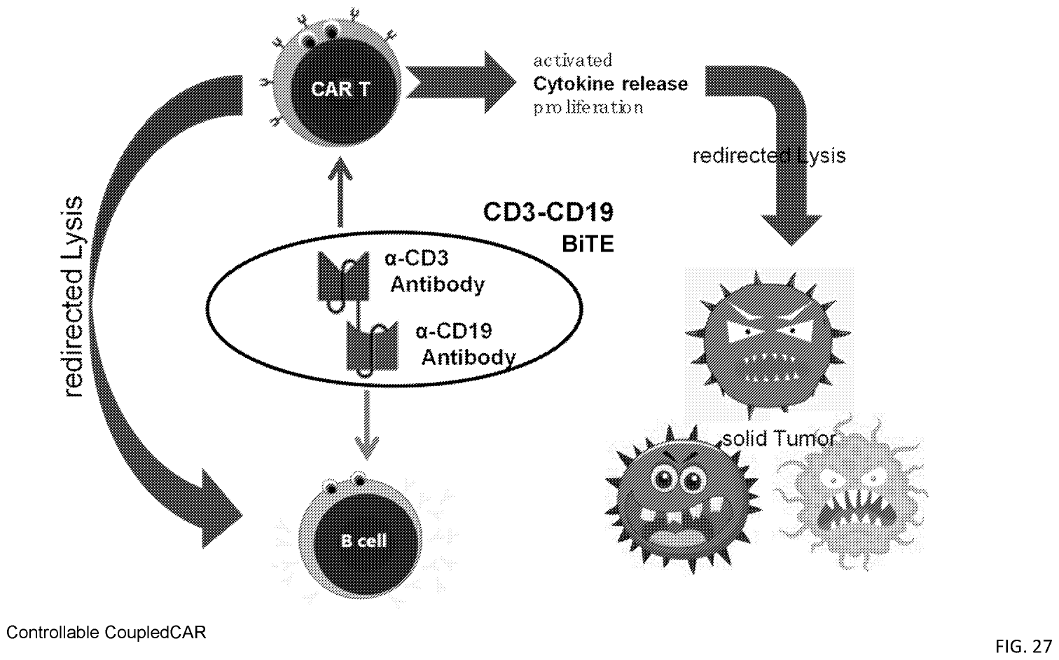

These results show that BiTEs targeting B cells caused and/or enhanced T cell response (e.g., activation, expansion, and cytokine release) of solid tumor CAR T cells in the presence of B cells, which is summarized in FIG. 27. As compared to a CoupledCAR® system including solid tumor CAR T and WBC CAR T cells, a CoupledCAR® system including BiTEs and solid tumor CAR T cells has advantages. BiTEs, such as Blinatumomab®, have been shown to be safe as well as effective and have a short half-life time. First, as compared to WBC CAR cells, BiTEs cost less and are easy to use. Second, given that BiTEs are antibodies, BiTEs' coupling effect is more manageable than WBC CAR T cells. Third, because they are more manageable, the CoupledCAR® system including BiTEs may cause fewer damages on B cells of a subject and can be more easily combined with other therapies, as compared to that of WBC CAR cells.

TABLE 7

| Identifiers | SEQ ID NO: |
|---|---|
| Linker | 1 |
| STAT3 association motif 1 | 2 |
| STAT3 association motif 2 | 3 |
| STAT5 association motif 3 | 4 |
| JAK-binding motif | 5 |
| CD3-humniazed CD19 | 6 |
| PAP scFv | 7 |
| Humanized CD19 scFv | 8 |
| GCC CAR | 9 |
| CD3-mCD19 | 10 |
| CD3-BCMA Bite | 11 |
| CD3-MAGE-A4 Bite | 12 |
| CD3-TSHR Bite | 13 |
| CD3-UPK2 Bite | 14 |
| CD3-GUCY2C Bite | 15 |
| CD3-CLDN18.2 Bite | 16 |
| CD3-ACPP Bite | 17 |
| CD3-ADAM12 Bite-1 | 18 |
| CD3-ADAM12 Bite-2 | 19 |
| CD3-MSLN Bite | 20 |
| CD3-CD205 Bite | 21 |
| CD3-GPC-3 Bite | 22 |

All publications, patents, and patent applications cited in this specification are incorporated herein by reference in their entirety as if each individual publication, patent, or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A method of enhancing activation of modified cells, the method comprising:
obtaining modified cells comprising chimeric antigen receptor (CAR) T cells, wherein the CAR of the CAR T cells comprises a binding domain, a transmembrane domain, and an intracellular domain, the binding domain binding a solid tumor antigen;
obtaining a bispecific antibody, wherein the bispecific antibody comprises a first binding domain binding CD3 and a second binding domain binding CD19, CD20, CD22, or BCMA;
contacting the CAR T cells with B cells and the bispecific antibody, thereby activating the modified cells, wherein level of activation of the CAR T cells is higher than level of activation in CAR T cells that are contacted with B cells without the bispecific antibody.

2. The method of claim 1, wherein the level of activation is measured based on a level of expression of CD69, CD25, or CD137 in the modified cells.

3. A method of expanding and/or activating modified cells, the method comprising:
obtaining modified cells comprising a binding molecule that binds a solid tumor antigen;
obtaining a polyspecific binding molecule (PBM), wherein the PBM comprises at least a first binding domain binding a T cell and at least a second binding domain binding an antigen of a white blood cell (WBC);
contacting the modified cells with a population of cells comprising an antigen of white blood cells (WBCs) and the PBM; and
allowing the modified cells to expand and/or to be activated,

4. The method of claim 3, wherein a level of expansion and/or activation in the modified cells is higher than a level of expansion and/or activation in modified cells that are contacted with the population of cells without the PBM.

5. The method of claim 1, wherein the solid tumor antigen comprises tMUC1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, B7-H3, CLDN18.2, MAGE A4, MSLN, CD205, or EGFR.

6. The method of claim 3, wherein the WBCs comprise a granulocyte, a monocyte, or a lymphocyte.

7. The method of claim 3, wherein the antigen of the WBC comprises CD19, CD22, CD20, BCMA, CD5, CD7, CD2, CD16, CD56, CD30, CD14, CD68, CD11b, CD18, CD169, CD1c, CD33, CD38, CD138, CD205, CD79a, CD79b, or CD13.

8. The method of claim 3, wherein the binding molecule is a CAR or a T cell receptor (TCR).

9. The method of claim 1, wherein the CAR comprises an antigen binding domain, a transmembrane domain, a co-stimulatory domain, and a CD3 zeta domain.

10. The method of claim 9, wherein the co-stimulatory domain comprises the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that binds CD83, or a combination thereof.

11. The method of claim 8, wherein the TCR binds CEA, gp100, MART-1, p53, MAGE-A3, or NY-ESO-1.

12. The method of claim 3, wherein the modified cells are T cells, NK cells, macrophages, or dendritic cells.

13. The method of claim 3, wherein activation of the activated modified cells is measured based on a level of expression of CD69, CD25, or CD137 in the modified cells.

14. The method of claim 3, wherein the expansion is measured based on numbers of modified cells or copy numbers of DNA encoding the CAR.

15. The method of claim 1, wherein the modified cells comprise an exogenous polynucleotide encoding a therapeutic agent comprising IL-1P, IL-2, IL-4, IL-5, IL-6, IL-7, IL-8, IL-10, IL-12, IL-13, IL-15, IL-17, IL-1Ra, IL-2R, IFNγ, MIP-In, MIP-IP, MCP-1, TNFα, GM-CSF, GCSF, CXCL9, CXCL10, CXCR factors, VEGF, RANTES, EOTAXIN, EGF, HGF, FGF-P, CD40, CD40L, or ferritin.

16. The method of claim 1, wherein the modified cells comprise a dominant negative form of PD-1, cytotoxic T lymphocyte antigen-4 (CTLA-4), B- and T-lymphocyte attenuator (BTLA), T-cell immunoglobulin mucin-3 (TIM-3), lymphocyte-activation protein 3 (LAG-3), T-cell immunoreceptor with Ig and ITIM domains (TIGIT), leukocyte-associated immunoglobulin-like receptor 1 (LAIRD, natural killer cell receptor 2B4 (2B4), or CD160.

17. The method of claim 1, wherein the PBM or bispecific antibody comprises SEQ ID NO: 6.

18. A pharmaceutical composition comprising an effective amount of the modified cells obtained by the method of claim 1.

19. A kit or pharmaceutical composition comprising a modified cell comprising
chimeric antigen receptor (CAR) T cells, wherein the CAR T cells comprise a binding domain, a transmembrane domain, and an intracellular domain, the binding domain binding a solid tumor antigen; and a bispecific antibody or a PBM.

20. The kit or pharmaceutical composition of claim 19, wherein the bispecific antibody comprises a first binding domain binding CD3 and a second binding domain binding CD19, CD20, CD22, or BCMA.

21. The kit or pharmaceutical composition of claim 21, wherein the PBM comprises at least a first binding domain binding a T cell and at least a second binding domain binding an antigen of a white blood cells (WBC).
